SEQUENCE LISTING (778 sequences)

SEQ ID NO: 1 - DNA, 50 bp, Artificial Sequence
  Description of Artificial Sequence/note = Synthetic Construct
gagttcgagaccagcctgggcaacatagcgagaccccgtctctaaaaaaa

SEQ ID NO: 2 - DNA, 50 bp, Artificial Sequence
  Description of Artificial Sequence/note = Synthetic Construct
ggagttcgagaccagcctgggcaacatagcgagaccccgtctctaaaaaa

SEQ ID NO: 3 - DNA, 50 bp, Artificial Sequence; identical to SEQ ID NO: 2
  Description of Artificial Sequence/note = Synthetic Construct

SEQ ID NO: 4 - DNA, 50 bp, Artificial Sequence
  Description of Artificial Sequence/note = Synthetic Construct
gaggcaggaggatcgcttgagcccaggagttcgaggctgcagtgagctat

SEQ ID NO: 5 - DNA, 50 bp, Artificial Sequence
  Description of Artificial Sequence/note = Synthetic Construct
ggagttcgagaccagcctgggcaacatggtgaaaccccgtctctacaaaa

SEQ ID NO: 6 - DNA, 50 bp, Artificial Sequence
  Description of Artificial Sequence/note = Synthetic Construct
tcacgaggtcaagagatcgagaccatcctggccaacatggtgaaaccccg

SEQ ID NO: 7 - DNA, 50 bp, Artificial Sequence
  Description of Artificial Sequence/note = Synthetic Construct
ccaacatggtgaaaccccgtctctactaaaaatacaaaaattagccgggc

SEQ ID NO: 8 - DNA, 50 bp, Artificial Sequence
  Description of Artificial Sequence/note = Synthetic Construct
ccagcctgaccaacatggagaaaccccgtctctactaaaaatacaaaaat

SEQ ID NO: 9 - DNA, 50 bp, Artificial Sequence
  Description of Artificial Sequence/note = Synthetic Construct
caacatggtgaaaccccgtctctactaaaaatacaaaaattagccgggcg

SEQ ID NO: 10 - DNA, 50 bp, Artificial Sequence; identical to SEQ ID NO: 7
  Description of Artificial Sequence/note = Synthetic Construct

SEQ ID NO: 11 - DNA, 50 bp, Artificial Sequence; identical to SEQ ID NO: 7
  Description of Artificial Sequence/note = Synthetic Construct

SEQ ID NO: 12 - DNA, 50 bp, Artificial Sequence
  Description of Artificial Sequence/note = Synthetic Construct
gagatcgagaccatcctggctaacacggtgaaaccccgtctctactaaaa

SEQ ID NO: 13 - DNA, 50 bp, Artificial Sequence
  Description of Artificial Sequence/note = Synthetic Construct
cgggcggatcacgaggtcaggagatcgagaccatcccggctaaaacggtg

SEQ ID NO: 14 - DNA, 50 bp, Artificial Sequence
  Description of Artificial Sequence/note = Synthetic Construct
gaaaccccgtctctactaaaactacaaaaaatagccgggcgtagtggcgg

SEQ ID NO: 15 - DNA, 50 bp, Artificial Sequence
  Description of Artificial Sequence/note = Synthetic Construct
agaccatcctggctaacaaggtgaaaccccgtctctactaaaaatacaaa

SEQ ID NO: 16 - DNA, 50 bp, Artificial Sequence; identical to SEQ ID NO: 15
  Description of Artificial Sequence/note = Synthetic Construct

SEQ ID NO: 17 - DNA, 50 bp, Artificial Sequence; identical to SEQ ID NO: 12
  Description of Artificial Sequence/note = Synthetic Construct

SEQ ID NO: 18 - DNA, 50 bp, Artificial Sequence
  Description of Artificial Sequence/note = Synthetic Construct
gagatcgagaccatcctggctaacaaggtgaaaccccgtctctactaaaa

SEQ ID NO: 19 - DNA, 50 bp, Artificial Sequence
  Description of Artificial Sequence/note = Synthetic Construct
cgcctgtagtcccagctactcggagaggctgaggcaggagaatggcgtga

SEQ ID NO: 20 - DNA, 50 bp, Artificial Sequence
  Description of Artificial Sequence/note = Synthetic Construct
accatcctggctaacacggtgaaaccccgtctctactaaaaatacaaaaa

SEQ ID NO: 21 - DNA, 50 bp, Artificial Sequence
  Description of Artificial Sequence/note = Synthetic Construct
atggatttgaggtttcctcccatctcctcattcggcggccctacgattaa

SEQ ID NO: 22 - DNA, 50 bp, Artificial Sequence
  Description of Artificial Sequence/note = Synthetic Construct
acggatttgaggtttcctcccatctcctcattcggcagccctacgattaa

SEQ ID NO: 23 - DNA, 50 bp, Artificial Sequence
  Description of Artificial Sequence/note = Synthetic Construct
ggcgtattgacttgctgtgtgcatcgggcaatgaacctattacggttaca

SEQ ID NO: 24 - DNA, 50 bp, Artificial Sequence; identical to SEQ ID NO: 12
  Description of Artificial Sequence/note = Synthetic Construct

SEQ ID NO: 25 - DNA, 50 bp, Artificial Sequence; identical to SEQ ID NO: 13
  Description of Artificial Sequence/note = Synthetic Construct

SEQ ID NO: 26 - DNA, 50 bp, Artificial Sequence; identical to SEQ ID NO: 12
  Description of Artificial Sequence/note = Synthetic Construct

SEQ ID NO: 27 - DNA, 50 bp, Artificial Sequence; identical to SEQ ID NO: 12
  Description of Artificial Sequence/note = Synthetic Construct

SEQ ID NO: 28 - DNA, 50 bp, Artificial Sequence; identical to SEQ ID NO: 20
  Description of Artificial Sequence/note = Synthetic Construct

SEQ ID NO: 29 - DNA, 50 bp, Artificial Sequence; identical to SEQ ID NO: 12
  Description of Artificial Sequence/note = Synthetic Construct

SEQ ID NO: 30 - DNA, 50 bp, Artificial Sequence; identical to SEQ ID NO: 12
  Description of Artificial Sequence/note = Synthetic Construct

SEQ ID NO: 31 - DNA, 50 bp, Artificial Sequence
  Description of Artificial Sequence/note = Synthetic Construct
gagatcgagaccatcctggctaacgcggtgaaaccccgcctctactaaaa

SEQ ID NO: 32 - DNA, 50 bp, Artificial Sequence
  Description of Artificial Sequence/note = Synthetic Construct
agatcgagaccatcctggctaacacggtgaaaccccgtctctactaaaaa

SEQ ID NO: 33 - DNA, 50 bp, Artificial Sequence; identical to SEQ ID NO: 32
  Description of Artificial Sequence/note = Synthetic Construct

SEQ ID NO: 34 - DNA, 50 bp, Artificial Sequence
  Description of Artificial Sequence/note = Synthetic Construct
gaccatcctggctaacacggtgaaaccccgtctctactaaaaatacaaaa

SEQ ID NO: 35 - DNA, 50 bp, Artificial Sequence
  Description of Artificial Sequence/note = Synthetic Construct
gatcgagaccatcctggctaacacagtgaaaccccgtctctactaaaaaa

SEQ ID NO: 36 - DNA, 50 bp, Artificial Sequence; identical to SEQ ID NO: 4
  Description of Artificial Sequence/note = Synthetic Construct

SEQ ID NO: 37 - DNA, 50 bp, Artificial Sequence
  Description of Artificial Sequence/note = Synthetic Construct
ggctctgccacttactagctgtgtgaccttgggcaagttacttaacctct

SEQ ID NO: 38 - DNA, 50 bp, Artificial Sequence
  Description of Artificial Sequence/note = Synthetic Construct
atcacatggacacaggaaggggaatatcacactctggggactgtggtggg

SEQ ID NO: 39 - DNA, 50 bp, Artificial Sequence
  Description of Artificial Sequence/note = Synthetic Construct
cctgtcggggggtggggggctaggggagggatagcattaggagaaatacc

SEQ ID NO: 40 - DNA, 50 bp, Artificial Sequence
  Description of Artificial Sequence/note = Synthetic Construct
tgggcttaatacctaggtgatgggatgatctgtgcagcaaaccaccatgg

SEQ ID NO: 41 - DNA, 50 bp, Artificial Sequence
  Description of Artificial Sequence/note = Synthetic Construct
tcgggtactatgcttattacctgggtgacgaaataatctgtacaccaaac

SEQ ID NO: 42 - DNA, 50 bp, Artificial Sequence
  Description of Artificial Sequence/note = Synthetic Construct
atctcagaaatcaccactaaagaacttattcatgtaaccaaacaccacct

SEQ ID NO: 43 - DNA, 50 bp, Artificial Sequence
  Description of Artificial Sequence/note = Synthetic Construct
aagtgggagctaagctatgggtacgcaaaggcatacagagtggtataatg

SEQ ID NO: 44 - DNA, 50 bp, Artificial Sequence
  Description of Artificial Sequence/note = Synthetic Construct
gggaagggtagtggggggttggtggggaggtggggatggttaatgggtac

SEQ ID NO: 45 - DNA, 50 bp, Artificial Sequence
  Description of Artificial Sequence/note = Synthetic Construct
atagggagaggttggttaatggatacaaaattacagctagataggaggaa

SEQ ID NO: 46 - DNA, 50 bp, Artificial Sequence
  Description of Artificial Sequence/note = Synthetic Construct
agatcttaagtgttctcaccacacacaaaaaaatggtaactatgtgaggt

SEQ ID NO: 47 - DNA, 50 bp, Artificial Sequence
  Description of Artificial Sequence/note = Synthetic Construct
ctgcacawgctctcttgcctgccgccatgtaagacgtgmctttgctcctc

SEQ ID NO: 48 - DNA, 50 bp, Artificial Sequence
  Description of Artificial Sequence/note = Synthetic Construct
tccccttggtgctgtcctcgtgatagtgagtgagttctcgtgagatctgg

SEQ ID NO: 49 - DNA, 50 bp, Artificial Sequence
  Description of Artificial Sequence/note = Synthetic Construct
gattaatggattaatgggttatcatgggagtgggactggtggctttataa

SEQ ID NO: 50 - DNA, 50 bp, Artificial Sequence
  Description of Artificial Sequence/note = Synthetic Construct
tgaggacacagtgagaaggcgccgtctacgaaccagggaatgagccctca

SEQ ID NO: 51 - DNA, 50 bp, Artificial Sequence
  Description of Artificial Sequence/note = Synthetic Construct
ggagaagacggccatctacaagccaaggagagaggcctcagaagaaacca

SEQ ID NO: 52 - DNA, 50 bp, Artificial Sequence
  Description of Artificial Sequence/note = Synthetic Construct
ccagcaaaccaccagaagctaggggagaggcatggaacagattctccctc

SEQ ID NO: 53 - DNA, 50 bp, Artificial Sequence
  Description of Artificial Sequence/note = Synthetic Construct
ggtcagagtcagagaaggagatgtgacgacggaagcagaggtcggagtga

SEQ ID NO: 54 - DNA, 50 bp, Artificial Sequence
  Description of Artificial Sequence/note = Synthetic Construct
gattccgtcttgncgncantcttgctgagagnctctcttgctggctttga

SEQ ID NO: 55 - DNA, 50 bp, Artificial Sequence
  Description of Artificial Sequence/note = Synthetic Construct
tgtagtcccctcccacattgaatagggctgacctgtgtgaccaatagaat

SEQ ID NO: 56 - DNA, 50 bp, Artificial Sequence
  Description of Artificial Sequence/note = Synthetic Construct
caagaggtgacttgggtgctgttaaaggcattcagttttaaaagggaagc

SEQ ID NO: 57 - DNA, 50 bp, Artificial Sequence
  Description of Artificial Sequence/note = Synthetic Construct
tctttttgattttacaggctcataggtggaaggaacttgccttgtctcag

SEQ ID NO: 58 - DNA, 50 bp, Artificial Sequence
  Description of Artificial Sequence/note = Synthetic Construct
agcctgatcatgtaacagaaannncaatagcgttctctggaaagaanacc

SEQ ID NO: 59 - DNA, 50 bp, Artificial Sequence
  Description of Artificial Sequence/note = Synthetic Construct
gggtgttgccaaaggaggttaacattggactcagtgggctggggagaggc

SEQ ID NO: 60 - DNA, 50 bp, Artificial Sequence
  Description of Artificial Sequence/note = Synthetic Construct
ttccagatgagattagcatttgaatcagcggactgagtaaagaagattgc

SEQ ID NO: 61 - DNA, 50 bp, Artificial Sequence
  Description of Artificial Sequence/note = Synthetic Construct
ctcaagactgcaacgtggaaatcctgctgntttwccagcctccaagcctt

SEQ ID NO: 62 - DNA, 50 bp, Artificial Sequence
  Description of Artificial Sequence/note = Synthetic Construct
ggctaggctatggtgtccagacgtttggtcaaacattagtctgggtgttt

SEQ ID NO: 63 - DNA, 50 bp, Artificial Sequence
  Description of Artificial Sequence/note = Synthetic Construct
caatgctcccagctgattaaagcctcttccttcatagaaccggtgtctaa

SEQ ID NO: 64 - DNA, 50 bp, Artificial Sequence
  Description of Artificial Sequence/note = Synthetic Construct
gcaaggagccccctgaccccttcttccaaacatactcttttgtctttgtc

SEQ ID NO: 65 - DNA, 50 bp, Artificial Sequence
  Description of Artificial Sequence/note = Synthetic Construct
atcctcctgtcccacccattggtctctcctgtcccttgattcctgcaaca

SEQ ID NO: 66 - DNA, 50 bp, Artificial Sequence
  Description of Artificial Sequence/note = Synthetic Construct
actcagaggctggtgggatcctccatatgctgaacgttggttccccgggc

SEQ ID NO: 67 - DNA, 50 bp, Artificial Sequence
  Description of Artificial Sequence/note = Synthetic Construct
aactccgtcactgtaatcccaatgtaaagcaagaattccaaaccaggaaa

SEQ ID NO: 68 - DNA, 50 bp, Artificial Sequence
  Description of Artificial Sequence/note = Synthetic Construct
gcttcattcttgaagtcagcgagaccaagaacccaccggaaggaaccaat

SEQ ID NO: 69 - DNA, 50 bp, Artificial Sequence
  Description of Artificial Sequence/note = Synthetic Construct
cttgtgtctttatttctacactctctcgtctccgcacacggggagaaaaa

SEQ ID NO: 70 - DNA, 50 bp, Artificial Sequence
  Description of Artificial Sequence/note = Synthetic Construct
aagcttcatctgtakttacagccgctccccatcactcgcattaccgcctg

SEQ ID NO: 71 - DNA, 50 bp, Artificial Sequence
  Description of Artificial Sequence/note = Synthetic Construct
tgatctgaggtggaacagtttcatcccgaaaccatccccgccccccggtc

SEQ ID NO: 72 - DNA, 50 bp, Artificial Sequence
  Description of Artificial Sequence/note = Synthetic Construct
aaaatccacggatgctcaagtccctgatataaaatggcgtagtatttgca

SEQ ID NO: 73 - DNA, 50 bp, Artificial Sequence
  Description of Artificial Sequence/note = Synthetic Construct
atgtggctaytgagcacttgaaatgtggytagtgcgactgaggaactgaa

SEQ ID NO: 74 - DNA, 50 bp, Artificial Sequence
  Description of Artificial Sequence/note = Synthetic Construct
ggacctcaagatctttaccctaaaacagttctgytgamyttcaccttggc

SEQ ID NO: 75 - DNA, 50 bp, Artificial Sequence
  Description of Artificial Sequence/note = Synthetic Construct
ttggtctccgcaaccccttatntcataacccggacattcctttccattga

SEQ ID NO: 76 - DNA, 50 bp, Artificial Sequence
  Description of Artificial Sequence/note = Synthetic Construct
cctccctctttcccctccagcccgcttttcccctttaaatattgaagccc

SEQ ID NO: 77 - DNA, 50 bp, Artificial Sequence
  Description of Artificial Sequence/note = Synthetic Construct
gtccccggaccagcagcatcagcatcacctgggaacttgttagaaatgca

SEQ ID NO: 78 - DNA, 50 bp, Artificial Sequence
  Description of Artificial Sequence/note = Synthetic Construct
tcagtattttttaaarctcyycaggtgattccaatgtgcagccaaggttg

SEQ ID NO: 79 - DNA, 50 bp, Artificial Sequence
  Description of Artificial Sequence/note = Synthetic Construct
aagtcgcagtttccaagaacctatcgacgacgttaagtgaggacttactg

SEQ ID NO: 80 - DNA, 50 bp, Artificial Sequence
  Description of Artificial Sequence/note = Synthetic Construct
aaaaatccgcgtataagtggacccacgcagttcaaacccgtgttgttcaa

SEQ ID NO: 81 - DNA, 50 bp, Artificial Sequence
  Description of Artificial Sequence/note = Synthetic Construct
gctgtgagacccctgatttcccacttcacacctctatatttctgtgtgtg

SEQ ID NO: 82 - DNA, 50 bp, Artificial Sequence
  Description of Artificial Sequence/note = Synthetic Construct
tgattttgcccttgtcctgtttcctcagaagcatgtgatctttgttctcc

SEQ ID NO: 83 - DNA, 50 bp, Artificial Sequence
  Description of Artificial Sequence/note = Synthetic Construct
acttgctggtttttgcggcttgtggggcatcacggaacctaccgacatgt

SEQ ID NO: 84 - DNA, 50 bp, Artificial Sequence
  Description of Artificial Sequence/note = Synthetic Construct
ccccacaacaaagaattatccggcccaaaatgtcgatagtgccaaggttg

SEQ ID NO: 85 - DNA, 50 bp, Artificial Sequence
  Description of Artificial Sequence/note = Synthetic Construct
sagcagaggraaaacatggtttgagagaggttttyctgmaayagragggc

SEQ ID NO: 86 - DNA, 50 bp, Artificial Sequence
  Description of Artificial Sequence/note = Synthetic Construct
cggtcagaagcacaggtnacaacctggngcttgcgactggcatctgaagt

SEQ ID NO: 87 - DNA, 50 bp, Artificial Sequence
  Description of Artificial Sequence/note = Synthetic Construct
tgagtctccccaaaagtggagcccttgtgatgacgagcacaggtccgcct

SEQ ID NO: 88 - DNA, 50 bp, Artificial Sequence
  Description of Artificial Sequence/note = Synthetic Construct
aagacgangaggatgaagacctttatgatgatccacttccacttaatgaa

SEQ ID NO: 89 - DNA, 50 bp, Artificial Sequence
  Description of Artificial Sequence/note = Synthetic Construct
ttttaagaaagtttacgaatttgtgttgggccgcattcaaagccatcctg

SEQ ID NO: 90 - DNA, 50 bp, Artificial Sequence
  Description of Artificial Sequence/note = Synthetic Construct
gatgaaaaggggatcctgtgcagaaaccacactacccatcagagaagcaa

SEQ ID NO: 91 - DNA, 50 bp, Artificial Sequence
  Description of Artificial Sequence/note = Synthetic Construct
ggcaggtcatagaaactagaactcctctcccccaaagcaagccataaaac

SEQ ID NO: 92 - DNA, 50 bp, Artificial Sequence
  Description of Artificial Sequence/note = Synthetic Construct
agggttcggtactatccgcggtttcaggcatccactgggggtcttggaac

SEQ ID NO: 93 - DNA, 50 bp, Artificial Sequence
  Description of Artificial Sequence/note = Synthetic Construct
cgcacctcaaactgcaaaagttacggccacagtgcgtgataagtgcttag

SEQ ID NO: 94 - DNA, 50 bp, Artificial Sequence
  Description of Artificial Sequence/note = Synthetic Construct
gaaattcttaataatttttgaacaaggggccccgcattttcattttgcac

SEQ ID NO: 95 - DNA, 50 bp, Artificial Sequence
  Description of Artificial Sequence/note = Synthetic Construct
tgttgttgtggacgcgctctcggggttsgaaccgayacaagarccttaca

SEQ ID NO: 96 - DNA, 50 bp, Artificial Sequence
  Description of Artificial Sequence/note = Synthetic Construct
tcttccttggcaatamtyrttgtctcagtgattggctttctgtgcagtga

SEQ ID NO: 97 - DNA, 50 bp, Artificial Sequence
  Description of Artificial Sequence/note = Synthetic Construct
ggggaaaggtggggaaaagattgagaaatcggatggttgccgtgtctgtg

SEQ ID NO: 98 - DNA, 50 bp, Artificial Sequence
  Description of Artificial Sequence/note = Synthetic Construct
gtggagatttcagccgctttgaggtcaatggtagaataggaaatatcttc

SEQ ID NO: 99 - DNA, 50 bp, Artificial Sequence
  Description of Artificial Sequence/note = Synthetic Construct
ggagtcaagaccccccagcccctcctccctcagactcatgagtccagacc

SEQ ID NO: 100 - DNA, 50 bp, Artificial Sequence
  Description of Artificial Sequence/note = Synthetic Construct
actcatggagggttagggttcaggttcgggttcgggttcgggttcgggtt

SEQ ID NO: 101 - DNA, 50 bp, Artificial Sequence
  Description of Artificial Sequence/note = Synthetic Construct
ggttctgagtgtttgtccctcacataggattccagaacactgctgctggg

SEQ ID NO: 102 - DNA, 50 bp, Artificial Sequence
  Description of Artificial Sequence/note = Synthetic Construct
tcacaatgcccctgtaggcagagcctagacaagagttacatcacctgggt

SEQ ID NO: 103 - DNA, 50 bp, Artificial Sequence
  Description of Artificial Sequence/note = Synthetic Construct
gggtccattcgatgatgatcacactggatttcattccataattctattcg

SEQ ID NO: 104 - DNA, 50 bp, Artificial Sequence
  Description of Artificial Sequence/note = Synthetic Construct
ccactgtctgtgctgtgtctttcaaaggtcagaagagattgnacctttgt

SEQ ID NO: 105 - DNA, 50 bp, Artificial Sequence
  Description of Artificial Sequence/note = Synthetic Construct
tgcrtttacaaacctttagctagacacagagcgctgattggtgcgttttt

SEQ ID NO: 106 - DNA, 50 bp, Artificial Sequence
  Description of Artificial Sequence/note = Synthetic Construct
cctgactcctgagtcacgttactgtcccactatacgttaagaggagggaa

SEQ ID NO: 107 - DNA, 50 bp, Artificial Sequence
  Description of Artificial Sequence/note = Synthetic Construct
aatatcaggaacaccggcatgtgcacttaggaccatgttttaatttttca

SEQ ID NO: 108 - DNA, 50 bp, Artificial Sequence
  Description of Artificial Sequence/note = Synthetic Construct
ggaatggaatggaatggaatggaatggaatggaatggaatggaatggaat

SEQ ID NO: 109 - DNA, 50 bp, Artificial Sequence
  Description of Artificial Sequence/note = Synthetic Construct
ggatgaggcaggaaagacagctgagggtcagaacccaggcaggtccaatg

SEQ ID NO: 110 - DNA, 50 bp, Artificial Sequence
  Description of Artificial Sequence/note = Synthetic Construct
actcgctgaaggctcagatgatcgttagcattttttagcaataaagtatt

SEQ ID NO: 111 - DNA, 50 bp, Artificial Sequence
  Description of Artificial Sequence/note = Synthetic Construct
taaagttacaccgagtgtgcctgcctctcctgcctccccttccacctcct

SEQ ID NO: 112 - DNA, 50 bp, Artificial Sequence
  Description of Artificial Sequence/note = Synthetic Construct
gggactcaggaggatgttgagggagacagaggggtgaagcgttgagacga

SEQ ID NO: 113 - DNA, 50 bp, Artificial Sequence
  Description of Artificial Sequence/note = Synthetic Construct
caggcggccagnctttcagggggaggatgaagtaggcctgggacaaaagc

SEQ ID NO: 114 - DNA, 50 bp, Artificial Sequence
  Description of Artificial Sequence/note = Synthetic Construct
aggactctacttctaatagtatggagaacactgatagtccttggcatgaa

SEQ ID NO: 115 - DNA, 50 bp, Artificial Sequence
  Description of Artificial Sequence/note = Synthetic Construct
ccctgtcacttgggttaagaccattggaagtacatcgattataaatctca

SEQ ID NO: 116 - DNA, 50 bp, Artificial Sequence
  Description of Artificial Sequence/note = Synthetic Construct
aacccaacagtatcaggtgctcagaaccgatgaagaagctcaagattgag

SEQ ID NO: 117 - DNA, 50 bp, Artificial Sequence
  Description of Artificial Sequence/note = Synthetic Construct
tggttaatgtgtaacaaggaggcagtaggccccaggtgtccagccagagg

SEQ ID NO: 118 - DNA, 50 bp, Artificial Sequence
  Description of Artificial Sequence/note = Synthetic Construct
aaaagtgaggacgagagtaagaactcccactaaaagtgaaaattctcaaa

SEQ ID NO: 119 - DNA, 50 bp, Artificial Sequence
  Description of Artificial Sequence/note = Synthetic Construct
cataccaccccccaaaaattttcactgccccaacacttcaacactatttt

SEQ ID NO: 120 - DNA, 50 bp, Artificial Sequence
  Description of Artificial Sequence/note = Synthetic Construct
ttgtaggatgctgtgtcataccctgtgccctaggattaatacaaaagctc

SEQ ID NO: 121 - DNA, 50 bp, Artificial Sequence
  Description of Artificial Sequence/note = Synthetic Construct
gcctccactctttatgaactcttaacctgtctcttctcattcctttgtca

SEQ ID NO: 122 - DNA, 50 bp, Artificial Sequence
  Description of Artificial Sequence/note = Synthetic Construct
ccggatcattcacagagttcaattcaattaacagtttaagcccccaaaaa

SEQ ID NO: 123 - DNA, 50 bp, Artificial Sequence
  Description of Artificial Sequence/note = Synthetic Construct
agagatcagacgaaacctgagaccagagactcattttcttctaaaatgct

SEQ ID NO: 124 - DNA, 50 bp, Artificial Sequence
  Description of Artificial Sequence/note = Synthetic Construct
acatgcatgtttgttcaatacgcatgcgtcaggaccaccttcatgaatat

SEQ ID NO: 125 - DNA, 50 bp, Artificial Sequence
  Description of Artificial Sequence/note = Synthetic Construct
caaccccccttatcttaactcaagctgacttcaactcttcaggcagagct

SEQ ID NO: 126 - DNA, 50 bp, Artificial Sequence
  Description of Artificial Sequence/note = Synthetic Construct
gccctcctgtctctcagtcccattctcccccgaggctagccatagaaact

SEQ ID NO: 127 - DNA, 50 bp, Artificial Sequence
  Description of Artificial Sequence/note = Synthetic Construct
tcttggagaagggatccttgttccccnctggcnctggtannccactgcag

SEQ ID NO: 128 - DNA, 50 bp, Artificial Sequence
  Description of Artificial Sequence/note = Synthetic Construct
aagcctaawttttcgtggccgtgtgacaaggaccccgtctttagctgaac

SEQ ID NO: 129 - DNA, 50 bp, Artificial Sequence
  Description of Artificial Sequence/note = Synthetic Construct
caacccttgccaaatgaagagaactgccttcncatgaagaattaantagt

SEQ ID NO: 130 - DNA, 50 bp, Artificial Sequence
  Description of Artificial Sequence/note = Synthetic Construct
gcacagagccatacaactaatacccctacttatagggttaggaatggcta

SEQ ID NO: 131 - DNA, 50 bp, Artificial Sequence
  Description of Artificial Sequence/note = Synthetic Construct
aaactggactaatgtccttgtcccaacaggtagatgctgatttaaataac

SEQ ID NO: 132 - DNA, 50 bp, Artificial Sequence
  Description of Artificial Sequence/note = Synthetic Construct
cacttcttcaagcatctcgacaactttttgcagggaaaacgcttccacaa

SEQ ID NO: 133 - DNA, 50 bp, Artificial Sequence
  Description of Artificial Sequence/note = Synthetic Construct
tggtatcatcgcttacaaaagtgtcttgaacttgatggagcttatgttga

SEQ ID NO: 134 - DNA, 50 bp, Artificial Sequence
  Description of Artificial Sequence/note = Synthetic Construct
aaacaaccccatcaaaaagtgggcaaaggatatgaacagacacttctcaa

SEQ ID NO: 135 - DNA, 50 bp, Artificial Sequence
  Description of Artificial Sequence/note = Synthetic Construct
gtgatggtttcacgggtgtatgcatatgtccaaactcatcaaattgtata

SEQ ID NO: 136 - DNA, 50 bp, Artificial Sequence
  Description of Artificial Sequence/note = Synthetic Construct
tcagtttgggaagatgaaaaagttctggagatggatggtggtgatggttg

SEQ ID NO: 137 - DNA, 50 bp, Artificial Sequence
  Description of Artificial Sequence/note = Synthetic Construct
agatagtggtgatggttgcacaactctgtgaatatactaaaaaccactga

SEQ ID NO: 138 - DNA, 50 bp, Artificial Sequence
  Description of Artificial Sequence/note = Synthetic Construct
atgttaataataggggaaactgtgtgngggnggggtgagggggtatatgg

SEQ ID NO: 139 - DNA, 50 bp, Artificial Sequence
  Description of Artificial Sequence/note = Synthetic Construct
ctgttggagtgggaggttacagataagcaaggggaggaggctagaatgat

SEQ ID NO: 140 - DNA, 50 bp, Artificial Sequence
  Description of Artificial Sequence/note = Synthetic Construct
tatttaggggtaanggggcatcatgtctgcaacttactctcaaatggttc

SEQ ID NO: 141 - DNA, 50 bp, Artificial Sequence
  Description of Artificial Sequence/note = Synthetic Construct
gcaggagggaagtgggtgtggctataaaagggcaacatgagggatccttg

SEQ ID NO: 142 - DNA, 50 bp, Artificial Sequence
  Description of Artificial Sequence/note = Synthetic Construct
gngngggggaagggaggtgggtgtggctataaaagggcagcacgagggat

SEQ ID NO: 143 - DNA, 50 bp, Artificial Sequence
  Description of Artificial Sequence/note = Synthetic Construct
agtggttgcctctggggagggtgantgactggaaaggggcatgagggaac

SEQ ID NO: 144 - DNA, 50 bp, Artificial Sequence
  Description of Artificial Sequence/note = Synthetic Construct
ggcaaaactaatctatgstgttagaagtcaggatagtggttacccttggg

SEQ ID NO: 145 - DNA, 50 bp, Artificial Sequence
  Description of Artificial Sequence/note = Synthetic Construct
ggtgttgggagagcctcagccggaatttcgtggacggacaagggcacaga

SEQ ID NO: 146 - DNA, 50 bp, Artificial Sequence
  Description of Artificial Sequence/note = Synthetic Construct
ctagaggtttgagcagcggggcactgaagaagcgagccacacccccatcg

SEQ ID NO: 147 - DNA, 50 bp, Artificial Sequence
  Description of Artificial Sequence/note = Synthetic Construct
atcctcctcaaccccatcggtctctctgattcctaaatcatccccaaaca

SEQ ID NO: 148 - DNA, 50 bp, Artificial Sequence
  Description of Artificial Sequence/note = Synthetic Construct
tttctctattgcaattcccctgtcttgatgaatcggctctgtctaggcag

SEQ ID NO: 149 - DNA, 50 bp, Artificial Sequence
  Description of Artificial Sequence/note = Synthetic Construct
taaactcctcgtgtgtgtccgtgtcctaaattttcctggcgcgngacgac

SEQ ID NO: 150 - DNA, 50 bp, Artificial Sequence
  Description of Artificial Sequence/note = Synthetic Construct
cctgtacctatcgcaatggtcctgaataaagtctgccttaccgtgcttta

SEQ ID NO: 151 - DNA, 50 bp, Artificial Sequence
  Description of Artificial Sequence/note = Synthetic Construct
gcccaaacccctttgtcttgtcacgttttcacaatttactactctttgtc

SEQ ID NO: 152 - DNA, 50 bp, Artificial Sequence
  Description of Artificial Sequence/note = Synthetic Construct
gcaacgtcaggaagttaccctatatggtctaaaaaggggaggcatgaata

SEQ ID NO: 153 - DNA, 50 bp, Artificial Sequence
  Description of Artificial Sequence/note = Synthetic Construct
tgccatggcaacgtcaggaagttaccctatatggtctaaaaaggggagga

SEQ ID NO: 154 - DNA, 50 bp, Artificial Sequence
  Description of Artificial Sequence/note = Synthetic Construct
tagcagagcacatctcccccgtaatgttctttggctttgttatcctatat

SEQ ID NO: 155 - DNA, 50 bp, Artificial Sequence
  Description of Artificial Sequence/note = Synthetic Construct
tggccctcttccaagtgtacttcgcttcctttcgttcctgctctaaaact

SEQ ID NO: 156 - DNA, 50 bp, Artificial Sequence
  Description of Artificial Sequence/note = Synthetic Construct
ttcaagctaccaacgtgatgtcactgaatgsggagttgggaaaagatata

SEQ ID NO: 157 - DNA, 50 bp, Artificial Sequence
  Description of Artificial Sequence/note = Synthetic Construct
atgtcactgaatgsggagttgggaagagatgcacagtagcacacyattat

SEQ ID NO: 158 - DNA, 50 bp, Artificial Sequence
  Description of Artificial Sequence/note = Synthetic Construct
acaatgtaacggctacagacacgacacacttttaagtttaatctgcatta

SEQ ID NO: 159 - DNA, 50 bp, Artificial Sequence
  Description of Artificial Sequence/note = Synthetic Construct
gaatatgcacatagtttactatggcacgcgtattcccattgcaatgctct

SEQ ID NO: 160 - DNA, 50 bp, Artificial Sequence
  Description of Artificial Sequence/note = Synthetic Construct
acatttgcctgacaactgtctcacraacctagctactgcaagagcctact

SEQ ID NO: 161 - DNA, 50 bp, Artificial Sequence
  Description of Artificial Sequence/note = Synthetic Construct
agactagctgaaacagggccagggcaaaagcacctctccataagacacac

SEQ ID NO: 162 - DNA, 50 bp, Artificial Sequence
  Description of Artificial Sequence/note = Synthetic Construct
cttgaacaccagaccaaattgaagactagctgaaacagggccagggcaaa

SEQ ID NO: 163 - DNA, 50 bp, Artificial Sequence
  Description of Artificial Sequence/note = Synthetic Construct
gcctcaacctcggcctataaagacttgaacaaacactaacatagtttcta

SEQ ID NO: 164 - DNA, 50 bp, Artificial Sequence
  Description of Artificial Sequence/note = Synthetic Construct
cacagaacaactccatccaaacccctgcactaagagacttgaccaaactc

SEQ ID NO: 165 - DNA, 50 bp, Artificial Sequence
  Description of Artificial Sequence/note = Synthetic Construct
tcttgagaacatgtatgtaatgggctgtatctgctcggctatataaaagg

SEQ ID NO: 166 - DNA, 50 bp, Artificial Sequence
  Description of Artificial Sequence/note = Synthetic Construct
aaccctgggcactgagtctctaatgagcttccctggtagacaacatttca

SEQ ID NO: 167 - DNA, 50 bp, Artificial Sequence
  Description of Artificial Sequence/note = Synthetic Construct
ttccctttgctgatcttgccgtgtatccttacnrtgtcgctgtaataaat

SEQ ID NO: 168 - DNA, 50 bp, Artificial Sequence
  Description of Artificial Sequence/note = Synthetic Construct
cccccaaattgtataagcttcaggccccacaaaacctggatctgcccctg

SEQ ID NO: 169 - DNA, 50 bp, Artificial Sequence
  Description of Artificial Sequence/note = Synthetic Construct
ttacaaaatcattgtcatatgaagaggcgatcaaagagtatgcagccaaa

SEQ ID NO: 170 - DNA, 50 bp, Artificial Sequence
  Description of Artificial Sequence/note = Synthetic Construct
tgttctgtctcaccggactcagacaagttggtaaccagtgcacagtgaac

SEQ ID NO: 171 - DNA, 50 bp, Artificial Sequence
  Description of Artificial Sequence/note = Synthetic Construct
tcngacccctattcctggtggttggcatagtgatgatctttgctattctc

SEQ ID NO: 172 - DNA, 50 bp, Artificial Sequence
  Description of Artificial Sequence/note = Synthetic Construct
ggcatgaagctcaattgcacatgtgcatgtttctcctttcataaatattc

SEQ ID NO: 173 - DNA, 50 bp, Artificial Sequence
  Description of Artificial Sequence/note = Synthetic Construct
ggtgacggggtacgactgggtttcaaacaacttatgtcaggcctaaaaat

SEQ ID NO: 174 - DNA, 50 bp, Artificial Sequence
  Description of Artificial Sequence/note = Synthetic Construct
gggggtatgggctctggattggttggtttgcatatgaaaggcgcgctccc

SEQ ID NO: 175 - DNA, 50 bp, Artificial Sequence
  Description of Artificial Sequence/note = Synthetic Construct
tggccgaagattcatttgatgaatccgatttttccgaaatagacgattct

SEQ ID NO: 176 - DNA, 50 bp, Artificial Sequence
  Description of Artificial Sequence/note = Synthetic Construct
tgttgccttaatcggctnctctgacacccggcagctcagctctctctcca

SEQ ID NO: 177 - DNA, 50 bp, Artificial Sequence
  Description of Artificial Sequence/note = Synthetic Construct
ggtgagcttccctggttggcaatactctntgcatgttgtcacacatcgtt

SEQ ID NO: 178 - DNA, 50 bp, Artificial Sequence
  Description of Artificial Sequence/note = Synthetic Construct
ccataggcttcaccagactgccaaaggggcccatggcacaaaaaaggtta

SEQ ID NO: 179 - DNA, 50 bp, Artificial Sequence
  Description of Artificial Sequence/note = Synthetic Construct
ntgcaaatgaccgngaaagtgctncaagtattgattttggggttacaaat

SEQ ID NO: 180 - DNA, 50 bp, Artificial Sequence
  Description of Artificial Sequence/note = Synthetic Construct
cacaaattctttgacactcttcccatcgaggagtggggtccgtntcctct

SEQ ID NO: 181 - DNA, 50 bp, Artificial Sequence
  Description of Artificial Sequence/note = Synthetic Construct
aataaaaactctcttcctccccagttcatctgcatctcgttattgggcca

SEQ ID NO: 182 - DNA, 50 bp, Artificial Sequence
  Description of Artificial Sequence/note = Synthetic Construct
ccagttcatctgcatctcgttattgggccacgagaataagcagcccgacc

SEQ ID NO: 183 - DNA, 50 bp, Artificial Sequence
  Description of Artificial Sequence/note = Synthetic Construct
gcagttatgggggatactcggctctttgcacatttggatnagagaagcat

SEQ ID NO: 184 - DNA, 50 bp, Artificial Sequence
  Description of Artificial Sequence/note = Synthetic Construct
cctggataaattcccctggggaacttgaggccccatatacacgaaattac

SEQ ID NO: 185 - DNA, 50 bp, Artificial Sequence
  Description of Artificial Sequence/note = Synthetic Construct
tttgttgggaactcagttacaaataaccctcaccataccagtactttctg

SEQ ID NO: 186 - DNA, 50 bp, Artificial Sequence
  Description of Artificial Sequence/note = Synthetic Construct
catgcttaaggagcccttcagcctgccactgcactgtgggaacactggcc

SEQ ID NO: 187 - DNA, 50 bp, Artificial Sequence
  Description of Artificial Sequence/note = Synthetic Construct
cgcctcctccacaaagaagaaccaaaatagcgagtagataatcacacttt

SEQ ID NO: 188 - DNA, 50 bp, Artificial Sequence
  Description of Artificial Sequence/note = Synthetic Construct
tgctccatctgcgagacgcacccttctatagaagtaaaattgccttgctg

SEQ ID NO: 189 - DNA, 50 bp, Artificial Sequence
  Description of Artificial Sequence/note = Synthetic Construct
gctgagagaccctttgtcctttggctcagtgttggttcttctttgcagca

SEQ ID NO: 190 - DNA, 50 bp, Artificial Sequence
  Description of Artificial Sequence/note = Synthetic Construct
cagtgtactctcatggcaaaactgctggtgagtgtaccctttctgcagaa

SEQ ID NO: 191 - DNA, 50 bp, Artificial Sequence
  Description of Artificial Sequence/note = Synthetic Construct
ctgcattgcagcccaacttctccctctgcccaatcctgcttccttccctt

SEQ ID NO: 192 - DNA, 50 bp, Artificial Sequence
  Description of Artificial Sequence/note = Synthetic Construct
ccaagaaccccaggtcagagaacacgaggcttgccaccatcttggaagtg

SEQ ID NO: 193 - DNA, 50 bp, Artificial Sequence
  Description of Artificial Sequence/note = Synthetic Construct
gcacgtaggcacagcttagtttagtctttacatagacaagactcctatat

SEQ ID NO: 194 - DNA, 50 bp, Artificial Sequence
  Description of Artificial Sequence/note = Synthetic Construct
tccgcaaccaatcagacgtttgcataggagtgtaactttgtaacttcact

SEQ ID NO: 195 - DNA, 50 bp, Artificial Sequence
  Description of Artificial Sequence/note = Synthetic Construct
ctttacttcgtcctcttcatttacatagggcgtaccccaagtaaccaatg

SEQ ID NO: 196 - DNA, 50 bp, Artificial Sequence
  Description of Artificial Sequence/note = Synthetic Construct
atcttctaccacatggctgcactggagtctctgaacctactctggttctg

SEQ ID NO: 197 - DNA, 50 bp, Artificial Sequence
  Description of Artificial Sequence/note = Synthetic Construct
tataaatttgttccgaccacgaggcatccctggagtctctctgaatctgc

SEQ ID NO: 198 - DNA, 50 bp, Artificial Sequence
  Description of Artificial Sequence/note = Synthetic Construct
caaccctggctgctgaaactgcctgttgtaacctgaaaccagttttatct

SEQ ID NO: 199 - DNA, 50 bp, Artificial Sequence
  Description of Artificial Sequence/note = Synthetic Construct
tctgcagcccaagaaccatcctataaaatctccagcaagcctttgtctcc

SEQ ID NO: 200 - DNA, 50 bp, Artificial Sequence
  Description of Artificial Sequence/note = Synthetic Construct
cataaatgctcctaaggaaaaatccaccgcggcgcgctcagtcctctctt

SEQ ID NO: 201 - DNA, 50 bp, Artificial Sequence
  Description of Artificial Sequence/note = Synthetic Construct
ttgactatgatgtgtaggaggggtagggctgctttagtaaaatgagtaag

SEQ ID NO: 202 - DNA, 50 bp, Artificial Sequence
  Description of Artificial Sequence/note = Synthetic Construct
gaaggcacccctcccgaggaaatctcaactgcacgacccctactacgccc

SEQ ID NO: 203 - DNA, 50 bp, Artificial Sequence
  Description of Artificial Sequence/note = Synthetic Construct
gttctcaaccttcctaatgccgcggccctttaatacagttcctgtgggtc

SEQ ID NO: 204 - DNA, 50 bp, Artificial Sequence
  Description of Artificial Sequence/note = Synthetic Construct
tgaaagatacactgtaaacacccacaaccamcttccctggagccccatca

SEQ ID NO: 205 - DNA, 50 bp, Artificial Sequence
  Description of Artificial Sequence/note = Synthetic Construct
tgtacatacggcttgcgcccaggctcactcgcgcccagagagagagtaaa

SEQ ID NO: 206 - DNA, 50 bp, Artificial Sequence
  Description of Artificial Sequence/note = Synthetic Construct
atgagagagctgctgaataaaaccatatttcacctgcctacggccccccg

SEQ ID NO: 207 - DNA, 50 bp, Artificial Sequence
  Description of Artificial Sequence/note = Synthetic Construct
agagagtgctcctgactgaaatcggccagaagcccctctcaggtttattc

SEQ ID NO: 208 - DNA, 50 bp, Artificial Sequence
  Description of Artificial Sequence/note = Synthetic Construct
gactgkwgagccgcttttcgtgtttctttcctctttctttaattcttaca

SEQ ID NO: 209 - DNA, 50 bp, Artificial Sequence
  Description of Artificial Sequence/note = Synthetic Construct
aataaattctgctcyacctcacccttcaatgtgtctgcatgcctaattct

SEQ ID NO: 210 - DNA, 50 bp, Artificial Sequence
  Description of Artificial Sequence/note = Synthetic Construct
gtaactngcttgataacgcaccctttattggcttccttcccttccctgtc

SEQ ID NO: 211 - DNA, 50 bp, Artificial Sequence
  Description of Artificial Sequence/note = Synthetic Construct
ctgcttyccttgactgtkawgggggcagccgrcaggttaataaargcttg

SEQ ID NO: 212 - DNA, 50 bp, Artificial Sequence
  Description of Artificial Sequence/note = Synthetic Construct
caataaagcttgcttgcctgactttgggtctcytcatcctttctctcggc

SEQ ID NO: 213 - DNA, 50 bp, Artificial Sequence
  Description of Artificial Sequence/note = Synthetic Construct
ttgagcagtaggatataaataactcccacatgcttagcgttccaataatg

SEQ ID NO: 214 - DNA, 50 bp, Artificial Sequence
  Description of Artificial Sequence/note = Synthetic Construct
gtgcyagctgnttagggccagcwgcwgtkacaaacctyycttggwgtstg

SEQ ID NO: 215 - DNA, 50 bp, Artificial Sequence
  Description of Artificial Sequence/note = Synthetic Construct
cctttaaaaaccacttgtaactgctgctaattggagtgtatattcagggc

SEQ ID NO: 216 - DNA, 50 bp, Artificial Sequence
  Description of Artificial Sequence/note = Synthetic Construct
aaaccttaacttctccactttggaacgctgaccccattcctttggagtct

SEQ ID NO: 217 - DNA, 50 bp, Artificial Sequence
  Description of Artificial Sequence/note = Synthetic Construct
gtcctgtccccccaaccatgtgagatagagccatctgggaatgagcttta

SEQ ID NO: 218 - DNA, 50 bp, Artificial Sequence
  Description of Artificial Sequence/note = Synthetic Construct
agcgggaatattagtggtgagttgttgctccctgtattgttgctgtggcc

SEQ ID NO: 219 - DNA, 50 bp, Artificial Sequence
  Description of Artificial Sequence/note = Synthetic Construct
acttactggctgtcgwgcggtgagcagtaccagctttggattcagttaca

SEQ ID NO: 220 - DNA, 50 bp, Artificial Sequence
  Description of Artificial Sequence/note = Synthetic Construct
aatggcagtcgtctcctgatctgttggccttaccatacctgaataataat

SEQ ID NO: 221 - DNA, 50 bp, Artificial Sequence
  Description of Artificial Sequence/note = Synthetic Construct
cttttcaatggcagtcgtctcctgatctgttggccttaccatacctsaat

SEQ ID NO: 222 - DNA, 50 bp, Artificial Sequence
  Description of Artificial Sequence/note = Synthetic Construct
aggggaacttgtggcagggaccagccttatcacactggtgcacctggtca

SEQ ID NO: 223 - DNA, 50 bp, Artificial Sequence
  Description of Artificial Sequence/note = Synthetic Construct
gagcccagtctgctaggcgggagagatgcctctaagttcttatctctggc

SEQ ID NO: 224 - DNA, 50 bp, Artificial Sequence
  Description of Artificial Sequence/note = Synthetic Construct
ggctcctgaaccttctcctaggcccatctgtgcacttccttgtaaaatcc

SEQ ID NO: 225 - DNA, 50 bp, Artificial Sequence
  Description of Artificial Sequence/note = Synthetic Construct
gccctgtccttggcctgcwtagcccagttttagcaagaatcctgctaagt

SEQ ID NO: 226 - DNA, 50 bp, Artificial Sequence
  Description of Artificial Sequence/note = Synthetic Construct
atccacctgccttttgtttcagnggagttgagttcaanctctaaccccta

SEQ ID NO: 227 - DNA, 50 bp, Artificial Sequence
  Description of Artificial Sequence/note = Synthetic Construct
gatgattcagctggtccttaatgaacaaaaggcmacccaacaagaaaatg

SEQ ID NO: 228 - DNA, 50 bp, Artificial Sequence
  Description of Artificial Sequence/note = Synthetic Construct
ttccacattgcaactaacctttaagaaactaccacttgtcgagttttggt

SEQ ID NO: 229 - DNA, 50 bp, Artificial Sequence
  Description of Artificial Sequence/note = Synthetic Construct
caccgcaactaacctttaagaaactaccacttgttgagttttggtgtagt

SEQ ID NO: 230 - DNA, 50 bp, Artificial Sequence
  Description of Artificial Sequence/note = Synthetic Construct
cagtggagttttccagaggctacatgacgtgtgatgtcgcaacagattga

SEQ ID NO: 231 - DNA, 50 bp, Artificial Sequence
  Description of Artificial Sequence/note = Synthetic Construct
taaaattctgtgggggaagtggaatggaaatacgagttcaaggagaaaaa

SEQ ID NO: 232 - DNA, 50 bp, Artificial Sequence
  Description of Artificial Sequence/note = Synthetic Construct
caatcttttggcttccctgggccacattggaagaagaattgtcttgggcc

SEQ ID NO: 233 - DNA, 50 bp, Artificial Sequence
  Description of Artificial Sequence/note = Synthetic Construct
ccgcatacgagttaaatgctcttatatttgcatttaaaactggcattgca

SEQ ID NO: 234 - DNA, 50 bp, Artificial Sequence
  Description of Artificial Sequence/note = Synthetic Construct
gcgagtatccccgtgcccgagggagcgtgacattaaatagcaaataaaaa

SEQ ID NO: 235 - DNA, 50 bp, Artificial Sequence
  Description of Artificial Sequence/note = Synthetic Construct
ctctccgctgrcagagagctttcttctttcacttattaaactttcactcc

SEQ ID NO: 236 - DNA, 50 bp, Artificial Sequence
  Description of Artificial Sequence/note = Synthetic Construct
tctcagtgtaattggtctgttactgcgcagtgggcatatgaacctgttgg

SEQ ID NO: 237 - DNA, 50 bp, Artificial Sequence
  Description of Artificial Sequence/note = Synthetic Construct
atcccgactcctgcgagaagtagctcaccgtgacaaagctgcctttgctt

SEQ ID NO: 238 - DNA, 50 bp, Artificial Sequence
  Description of Artificial Sequence/note = Synthetic Construct
tctctcaagaataccccaaaaattaagtttttctttttccaaggtgccca

SEQ ID NO: 239 - DNA, 50 bp, Artificial Sequence
  Description of Artificial Sequence/note = Synthetic Construct
cctgtgatctcgccctgcctccacttgccttgtgatattctattaccytg

SEQ ID NO: 240 - DNA, 50 bp, Artificial Sequence
  Description of Artificial Sequence/note = Synthetic Construct
ttcatccccatgtgaccatctcacctcataatcaaatgaccctaaatccc

SEQ ID NO: 241 - DNA, 50 bp, Artificial Sequence
  Description of Artificial Sequence/note = Synthetic Construct
ggcgactggccaaggagaagcacccctctgcgcagaagtaaaattgcttt

SEQ ID NO: 242 - DNA, 50 bp, Artificial Sequence
  Description of Artificial Sequence/note = Synthetic Construct
ccacactcgcgatggccccctggtcccactttctctctcaaactgtcttt

SEQ ID NO: 243 - DNA, 50 bp, Artificial Sequence
  Description of Artificial Sequence/note = Synthetic Construct
tttgcagcctccatacttagcgttggccccctggacccactttctctctc

SEQ ID NO: 244 - DNA, 50 bp, Artificial Sequence
  Description of Artificial Sequence/note = Synthetic Construct
gtgggacaagaacttgggaatcagtgcacaagccagacttggcctgggaa

SEQ ID NO: 245 - DNA, 50 bp, Artificial Sequence
  Description of Artificial Sequence/note = Synthetic Construct
attgatccccacccttcacctattttacatatacccaccctttcctaatt

SEQ ID NO: 246 - DNA, 50 bp, Artificial Sequence
  Description of Artificial Sequence/note = Synthetic Construct
ttaatcaatctgccttntgtcagtgatttttcagcgaaccttcagggggc

SEQ ID NO: 247 - DNA, 50 bp, Artificial Sequence
  Description of Artificial Sequence/note = Synthetic Construct
ctttttttctctcttggtccgatccgtgtctctcwctcgccgcgggcwgc

SEQ ID NO: 248 - DNA, 50 bp, Artificial Sequence
  Description of Artificial Sequence/note = Synthetic Construct
tttctcttttgcaaaacccatcgtcacagtgattgrcttactgcgcgcgg

SEQ ID NO: 249 - DNA, 50 bp, Artificial Sequence
  Description of Artificial Sequence/note = Synthetic Construct
accctttcctgactgattctctctgaataatgcccacctgcgcactggga

SEQ ID NO: 250 - DNA, 50 bp, Artificial Sequence
  Description of Artificial Sequence/note = Synthetic Construct
ccgacccgccccacaagtgtttacatcagatgcttttgtgcagatgaggg

SEQ ID NO: 251 - DNA, 50 bp, Artificial Sequence
  Description of Artificial Sequence/note = Synthetic Construct
cgcttgcccactgtcycctttctactggttctgcttaycyctccctataa

SEQ ID NO: 252 - DNA, 50 bp, Artificial Sequence
  Description of Artificial Sequence/note = Synthetic Construct
ttctgcctgaactttgagatgcttgcagatcttatggtcagagcgttctc

SEQ ID NO: 253 - DNA, 50 bp, Artificial Sequence
  Description of Artificial Sequence/note = Synthetic Construct
tatctaccccttcctataaaagtccaaggcaaaaccaccctgccgagaca

SEQ ID NO: 254 - DNA, 50 bp, Artificial Sequence
  Description of Artificial Sequence/note = Synthetic Construct
gccctgggttcctacgtaagcaaaccgaaacctaactcagncgtttctta

SEQ ID NO: 255 - DNA, 50 bp, Artificial Sequence
  Description of Artificial Sequence/note = Synthetic Construct
cacagatgcatgagggagcccagccgagaccagaagaaccacccagctga

SEQ ID NO: 256 - DNA, 50 bp, Artificial Sequence
  Description of Artificial Sequence/note = Synthetic Construct
gaacccagaaacaaatccatacatytacagcgaactcattttcgacaaag

SEQ ID NO: 257 - DNA, 50 bp, Artificial Sequence
  Description of Artificial Sequence/note = Synthetic Construct
atgtaagtccccaataaaccctatgtctcatttgctggctctgggtctct

SEQ ID NO: 258 - DNA, 50 bp, Artificial Sequence
  Description of Artificial Sequence/note = Synthetic Construct
gcacaacgacgaaatcgcctaacgacgcatttctcagaacgtatccccgt

SEQ ID NO: 259 - DNA, 50 bp, Artificial Sequence
  Description of Artificial Sequence/note = Synthetic Construct
tagtgacacctttgctttctgatggttcaatgtacacaaactttgtttca

SEQ ID NO: 260 - DNA, 50 bp, Artificial Sequence
  Description of Artificial Sequence/note = Synthetic Construct
cggattttcagatttgggatgctcaaccggtaagtataatgcaaatattc

SEQ ID NO: 261 - DNA, 50 bp, Artificial Sequence
  Description of Artificial Sequence/note = Synthetic Construct
nctgccagncaacnacagnttgtgcacctngntggcaraganactgacac

SEQ ID NO: 262 - DNA, 50 bp, Artificial Sequence
  Description of Artificial Sequence/note = Synthetic Construct
cgctgttgctagccccggggtgcttcaccatcccttgttggtttccctta

SEQ ID NO: 263 - DNA, 50 bp, Artificial Sequence
  Description of Artificial Sequence/note = Synthetic Construct
aagtcagcttcaaataaagaccctgcacaaagcctcggcccggtgaaaac

SEQ ID NO: 264 - DNA, 50 bp, Artificial Sequence
  Description of Artificial Sequence/note = Synthetic Construct
gacagccanacaatagacagcctgtcaataganatagccacacaataata

SEQ ID NO: 265 - DNA, 50 bp, Artificial Sequence
  Description of Artificial Sequence/note = Synthetic Construct
aagaatctgaacagcagcccttgagtcccagatcttccctctgacatagt

SEQ ID NO: 266 - DNA, 50 bp, Artificial Sequence
  Description of Artificial Sequence/note = Synthetic Construct
aatctacccacctgctttagccacarctggtkyytacccakggayacctc

SEQ ID NO: 267 - DNA, 50 bp, Artificial Sequence
  Description of Artificial Sequence/note = Synthetic Construct
aagaaacatawtcacattcaarggagtcccaatatggctatcagcagatt

SEQ ID NO: 268 - DNA, 50 bp, Artificial Sequence
  Description of Artificial Sequence/note = Synthetic Construct
agtggmaatctcatcagcccagggatctracaggagaaggtcttcctccc

SEQ ID NO: 269 - DNA, 50 bp, Artificial Sequence
  Description of Artificial Sequence/note = Synthetic Construct
yacatcmatagaaaaggtctgagagagycccagaatccctagccaggctg

SEQ ID NO: 270 - DNA, 50 bp, Artificial Sequence
  Description of Artificial Sequence/note = Synthetic Construct
gtcgcgctacgctgatangattnancataccctanatgctcggcgactgc

SEQ ID NO: 271 - DNA, 50 bp, Artificial Sequence
  Description of Artificial Sequence/note = Synthetic Construct
cactcagtgcgaamagcattatacctgggggcatttgttgaaaacawtta

SEQ ID NO: 272 - DNA, 50 bp, Artificial Sequence
  Description of Artificial Sequence/note = Synthetic Construct
tgaaagtggacttggattagttgtaaatgtatattgcaaactctagggca

SEQ ID NO: 273 - DNA, 50 bp, Artificial Sequence
  Description of Artificial Sequence/note = Synthetic Construct
ctgacacctacagctacagcaaacagtaaacacagtctaactcttagcca

SEQ ID NO: 274 - DNA, 50 bp, Artificial Sequence
  Description of Artificial Sequence/note = Synthetic Construct
accacagccactggaaagagtggggaaaatcccggaaaggagagagccag

SEQ ID NO: 275 - DNA, 50 bp, Artificial Sequence
  Description of Artificial Sequence/note = Synthetic Construct
acaaaaatatccagcacccaacaaggtaaaattcacaatgtctggcatcc

SEQ ID NO: 276 - DNA, 50 bp, Artificial Sequence
  Description of Artificial Sequence/note = Synthetic Construct
tcgtgaccttgggytaggcaawgatttcttagatatgacacmaaaagcac

SEQ ID NO: 277 - DNA, 50 bp, Artificial Sequence
  Description of Artificial Sequence/note = Synthetic Construct
aagctctgaataaatagcctttgcttgttctcatttggktggtcttcatt

SEQ ID NO: 278 - DNA, 50 bp, Artificial Sequence
  Description of Artificial Sequence/note = Synthetic Construct
cctcgctgcarcgagcaataaacccaacttgttcaaccacaggtgtgttc

SEQ ID NO: 279 - DNA, 50 bp, Artificial Sequence
  Description of Artificial Sequence/note = Synthetic Construct
acagcaaccaaaacgagattacggagtagactggacataagcaacacact

SEQ ID NO: 280 - DNA, 50 bp, Artificial Sequence
  Description of Artificial Sequence/note = Synthetic Construct
ataatgacaattttccaacagatggcagtaaagtgtcttgaggaaggggc

SEQ ID NO: 281 - DNA, 50 bp, Artificial Sequence
  Description of Artificial Sequence/note = Synthetic Construct
cctgtacttcttcaaatgataaaaagcttcatcgctaccttagttcacca

SEQ ID NO: 282 - DNA, 50 bp, Artificial Sequence
  Description of Artificial Sequence/note = Synthetic Construct
tgccttccaagcaatgaatatgctcaattnaaatcatatgctcgtgattg

SEQ ID NO: 283 - DNA, 50 bp, Artificial Sequence
  Description of Artificial Sequence/note = Synthetic Construct
gaaattgcctaatgacgcatttctcagaacgtatccccgtcgttaagcga

SEQ ID NO: 284 - DNA, 50 bp, Artificial Sequence
  Description of Artificial Sequence/note = Synthetic Construct
tcctgcaagctccattcatggtaagtgcyctatacaggtgtaccattttt

SEQ ID NO: 285 - DNA, 50 bp, Artificial Sequence
  Description of Artificial Sequence/note = Synthetic Construct
tgtgtctgtggctcgcgtttttcccggacatgccctaaagctggcttaat

SEQ ID NO: 286 - DNA, 50 bp, Artificial Sequence
  Description of Artificial Sequence/note = Synthetic Construct
cgtgttaatttcyattacatggrgagcccaggaacctgtggtcnntaaca

SEQ ID NO: 287 - DNA, 50 bp, Artificial Sequence
  Description of Artificial Sequence/note = Synthetic Construct
cctgtacttcttccccctaagctagctttggaataaaaagtcactttctt

SEQ ID NO: 288 - DNA, 50 bp, Artificial Sequence
  Description of Artificial Sequence/note = Synthetic Construct
cagactgaaggctgcactgtyggcttccctacttttgaggttttgggact

SEQ ID NO: 289 - DNA, 50 bp, Artificial Sequence
  Description of Artificial Sequence/note = Synthetic Construct
gnagggatggggactgcttttcgtnataagccttgtagnactatttgact

SEQ ID NO: 290 - DNA, 50 bp, Artificial Sequence
  Description of Artificial Sequence/note = Synthetic Construct
ctgggccccttagatcaggtatccagagatttttactcctccggtgctag

SEQ ID NO: 291 - DNA, 50 bp, Artificial Sequence
  Description of Artificial Sequence/note = Synthetic Construct
ttccttcccccactgtggaaaaagccagttttgcntcyatttgcaaattc

SEQ ID NO: 292 - DNA, 50 bp, Artificial Sequence
  Description of Artificial Sequence/note = Synthetic Construct
gggaatgtacctntgttgactttgctatttactatttgattagggcccag

SEQ ID NO: 293 - DNA, 50 bp, Artificial Sequence
  Description of Artificial Sequence/note = Synthetic Construct
acgttttctcaccgatatcacactgcatatgaacaagctaaatttgaagc

SEQ ID NO: 294 - DNA, 50 bp, Artificial Sequence
  Description of Artificial Sequence/note = Synthetic Construct
ttaaggtaggctaggctaagctatgatgttcggtaggttaggtgtattaa

SEQ ID NO: 295 - DNA, 50 bp, Artificial Sequence
  Description of Artificial Sequence/note = Synthetic Construct
ggtttctactgaatgtgtatcgctttcgcaccatcgtaaagttgaaaaat

SEQ ID NO: 296 - DNA, 50 bp, Artificial Sequence
  Description of Artificial Sequence/note = Synthetic Construct
gtttaccctcgtgatcgcgcggctgactgggarctgcggytcactgycgc

SEQ ID NO: 297 - DNA, 50 bp, Artificial Sequence
  Description of Artificial Sequence/note = Synthetic Construct
atctcccatctgctagcatttgattaataaagctgctttcctttcaccac

SEQ ID NO: 298 - DNA, 50 bp, Artificial Sequence
  Description of Artificial Sequence/note = Synthetic Construct
atgacagttgatgagcagttagttgcattcaaaggatattgcccatttcg

SEQ ID NO: 299 - DNA, 50 bp, Artificial Sequence
  Description of Artificial Sequence/note = Synthetic Construct
gcgcctgacagacctgttgctgcacacatctgtactcttcaatcaacaaa

SEQ ID NO: 300 - DNA, 50 bp, Artificial Sequence
  Description of Artificial Sequence/note = Synthetic Construct
accacccctggtcattaaggagctaccctgtctccattagahagagcagg

SEQ ID NO: 301 - DNA, 50 bp, Artificial Sequence
  Description of Artificial Sequence/note = Synthetic Construct
gagcagagccccagccgacccgcgatggacatgtagcatgagcaagaaat

SEQ ID NO: 302 - DNA, 50 bp, Artificial Sequence
  Description of Artificial Sequence/note = Synthetic Construct
aggggtagtggctgctccttatatctgctattcctatattctttagagtt

SEQ ID NO: 303 - DNA, 50 bp, Artificial Sequence
  Description of Artificial Sequence/note = Synthetic Construct
caataaagctcctcttcgccttgctcaccctccacttgtccgcgtacctc

SEQ ID NO: 304 - DNA, 50 bp, Artificial Sequence
  Description of Artificial Sequence/note = Synthetic Construct
tctcctctgagctgttctatcgctcaataaagctcctcttcatcttgctc

SEQ ID NO: 305 - DNA, 50 bp, Artificial Sequence
  Description of Artificial Sequence/note = Synthetic Construct
aggatggccagaggacaaagrgggcagagagacaatgggacwggatgacc

SEQ ID NO: 306 - DNA, 50 bp, Artificial Sequence
  Description of Artificial Sequence/note = Synthetic Construct
gcctgggacagtcctggtttatrcctgttgtcctggcgtaattattaata

SEQ ID NO: 307 - DNA, 50 bp, Artificial Sequence
  Description of Artificial Sequence/note = Synthetic Construct
gaggggnaaccacacaaaaagagnaggctaataagttggccaaaataagc

SEQ ID NO: 308 - DNA, 50 bp, Artificial Sequence
  Description of Artificial Sequence/note = Synthetic Construct
tttctcccgctgcaaaatctcggtgtsgatgtttggttttactgcgccgg

SEQ ID NO: 309 - DNA, 50 bp, Artificial Sequence
  Description of Artificial Sequence/note = Synthetic Construct
tctctgacccaggagtctcgtgtcttctgccagcatccatgaaactgtgg

SEQ ID NO: 310 - DNA, 50 bp, Artificial Sequence
  Description of Artificial Sequence/note = Synthetic Construct
tctctgacccaggagtctcatgtcttctgccagcatccatgaaactgtgg

SEQ ID NO: 311 - DNA, 50 bp, Artificial Sequence
  Description of Artificial Sequence/note = Synthetic Construct
tgcttggatgtcctgttgatagtagccttaattaaatgctntatgagaca

SEQ ID NO: 312 - DNA, 50 bp, Artificial Sequence
  Description of Artificial Sequence/note = Synthetic Construct
gtgtcgttttatctaaatcggcgcgaggaccaaggaccctggtgttcctc

SEQ ID NO: 313 - DNA, 50 bp, Artificial Sequence
  Description of Artificial Sequence/note = Synthetic Construct
ctccaaatggtgctgcagaccgaaccacacatagacacgccattcttcca

SEQ ID NO: 314 - DNA, 50 bp, Artificial Sequence
  Description of Artificial Sequence/note = Synthetic Construct
gagatsaaatcaaaatcattgacaggctcagggaaaatgccggcttcagc

SEQ ID NO: 315 - DNA, 50 bp, Artificial Sequence
  Description of Artificial Sequence/note = Synthetic Construct
tagacacaggnaagagacctgggaagcttnagtagccaccgtgtaagccc

SEQ ID NO: 316 - DNA, 50 bp, Artificial Sequence
  Description of Artificial Sequence/note = Synthetic Construct
ttcgctccaacctcaccctttgtgtccatgctccttaattttcttggtcg

SEQ ID NO: 317 - DNA, 50 bp, Artificial Sequence
  Description of Artificial Sequence/note = Synthetic Construct
ctragracccttaaaccagcctcrrgaraartcctaactgctgttnccta

SEQ ID NO: 318 - DNA, 50 bp, Artificial Sequence
  Description of Artificial Sequence/note = Synthetic Construct
cttctttctttggaatcccaactggccccatctcagganggtttggggya

SEQ ID NO: 319 - DNA, 50 bp, Artificial Sequence
  Description of Artificial Sequence/note = Synthetic Construct
ttcytttgcaataaattrctctatgctgcatctcctttgctgtgtgtctc

SEQ ID NO: 320 - DNA, 50 bp, Artificial Sequence
  Description of Artificial Sequence/note = Synthetic Construct
gtgtgtcttcccaggtcaatcctcacatttggcttccaataaacctttat

SEQ ID NO: 321 - DNA, 50 bp, Artificial Sequence
  Description of Artificial Sequence/note = Synthetic Construct
gtctcccggttcgcgarctgtwctttctctyattgtatgcacaataaact

SEQ ID NO: 322 - DNA, 50 bp, Artificial Sequence
  Description of Artificial Sequence/note = Synthetic Construct
taaatgacaccatrgggatgcaatcagcaaaatccagactgtgggaaact

SEQ ID NO: 323 - DNA, 50 bp, Artificial Sequence
  Description of Artificial Sequence/note = Synthetic Construct
atggagcagagctgccataccagccctggactgcctacctctagacttct

SEQ ID NO: 324 - DNA, 50 bp, Artificial Sequence
  Description of Artificial Sequence/note = Synthetic Construct
caagacatgatgctactccaagaataccgacggctccaggaacagcagtc

SEQ ID NO: 325 - DNA, 50 bp, Artificial Sequence
  Description of Artificial Sequence/note = Synthetic Construct
aaactcatttggcagcaaaacctgacctgaactgatatgaggctatttat

SEQ ID NO: 326 - DNA, 50 bp, Artificial Sequence
  Description of Artificial Sequence/note = Synthetic Construct
aatttaaggaggcactcactctcagggtcgtgcaagtgcagggtcggcat

SEQ ID NO: 327 - DNA, 50 bp, Artificial Sequence
  Description of Artificial Sequence/note = Synthetic Construct
gcccacctcctgtctccttgctggccggttttgcaataaagcctttcttt

SEQ ID NO: 328 - DNA, 50 bp, Artificial Sequence
  Description of Artificial Sequence/note = Synthetic Construct
tctggcattaagctggtcccccacytyyrcaggttttntgctggatataa

SEQ ID NO: 329 - DNA, 50 bp, Artificial Sequence
  Description of Artificial Sequence/note = Synthetic Construct
gctttcaacttgatgtcagtggattccttcgaatcagtaatgtctctatg

SEQ ID NO: 330 - DNA, 50 bp, Artificial Sequence
  Description of Artificial Sequence/note = Synthetic Construct
aatacggttcgtctgctcataactgttatacccgtgcgactgtcattagt

SEQ ID NO: 331 - DNA, 50 bp, Artificial Sequence
  Description of Artificial Sequence/note = Synthetic Construct
ctcaggctccagtatgagtngacactgcacagttrctgatcctgtattta

SEQ ID NO: 332 - DNA, 50 bp, Artificial Sequence
  Description of Artificial Sequence/note = Synthetic Construct
tcttgccaccacgnggagagagcctgcctgagaatgaagccaacacagag

SEQ ID NO: 333 - DNA, 50 bp, Artificial Sequence
  Description of Artificial Sequence/note = Synthetic Construct
cccttggaccagtctaaagcaccacattaacatcttatatgtagtccttg

SEQ ID NO: 334 - DNA, 50 bp, Artificial Sequence
  Description of Artificial Sequence/note = Synthetic Construct
cgctgcatacctgtgtctgagtactcatttcatccatcggtcggccaggg

SEQ ID NO: 335 - DNA, 50 bp, Artificial Sequence
  Description of Artificial Sequence/note = Synthetic Construct
acacagacgtggcttctgtttgtaagtccctattaaatgtttctttctga

SEQ ID NO: 336 - DNA, 50 bp, Artificial Sequence
  Description of Artificial Sequence/note = Synthetic Construct
tccttctgcgtttgggggtcattttgcatatacggccctttcacgaaaca

SEQ ID NO: 337 - DNA, 50 bp, Artificial Sequence
  Description of Artificial Sequence/note = Synthetic Construct
ttcgttttacaccgaaggctgcatctccccggtttgcaaactgttcactg

SEQ ID NO: 338 - DNA, 50 bp, Artificial Sequence
  Description of Artificial Sequence/note = Synthetic Construct
cagttcatttcagcaaaccttcagaggggacagaggggaagctttccttt

SEQ ID NO: 339 - DNA, 50 bp, Artificial Sequence
  Description of Artificial Sequence/note = Synthetic Construct
taatcattctcctctgtgattcccccatgctatgcacgttaaaataaatt

SEQ ID NO: 340 - DNA, 50 bp, Artificial Sequence
  Description of Artificial Sequence/note = Synthetic Construct
tgccttttgtcagttgatttttcagcgaaccttcagagggcgaaggggaa

SEQ ID NO: 341 - DNA, 50 bp, Artificial Sequence
  Description of Artificial Sequence/note = Synthetic Construct
ctctttctttattgcaatgccatggtctttgtctgtgcagcgggcaggaa

SEQ ID NO: 342 - DNA, 50 bp, Artificial Sequence
  Description of Artificial Sequence/note = Synthetic Construct
gtagaagccccaaacccymttggcgcaactcwctctcttgagtatgcccg

SEQ ID NO: 343 - DNA, 50 bp, Artificial Sequence
  Description of Artificial Sequence/note = Synthetic Construct
tcccccctccagaccttcacttccccagctcctcccacaattgtataagg

SEQ ID NO: 344 - DNA, 50 bp, Artificial Sequence
  Description of Artificial Sequence/note = Synthetic Construct
tctctgttaaaataactggtgtggtttctgtcttctcctgactggaccct

SEQ ID NO: 345 - DNA, 50 bp, Artificial Sequence
  Description of Artificial Sequence/note = Synthetic Construct
tctttgaagagagagcgcctttggtctatgccagagactatctcttccca

SEQ ID NO: 346 - DNA, 50 bp, Artificial Sequence
  Description of Artificial Sequence/note = Synthetic Construct
gtgcattgtgaatctccaagaggggaaatatagtatgcagtrtttcccaa

SEQ ID NO: 347 - DNA, 50 bp, Artificial Sequence
  Description of Artificial Sequence/note = Synthetic Construct
ttaacatctctgaaatcgggatgcatcttacaatcgatggcatgtcatag

SEQ ID NO: 348 - DNA, 50 bp, Artificial Sequence
  Description of Artificial Sequence/note = Synthetic Construct
acaacggcagagttgagtagttgcgacagagaccgtatggcccgcaaagc

SEQ ID NO: 349 - DNA, 50 bp, Artificial Sequence; identical to SEQ ID NO: 348
  Description of Artificial Sequence/note = Synthetic Construct

SEQ ID NO: 350 - DNA, 50 bp, Artificial Sequence
  Description of Artificial Sequence/note = Synthetic Construct
atctgctcttcgccttgcccagagaccccactgtgaattaccatttggag

SEQ ID NO: 351 - DNA, 50 bp, Artificial Sequence
  Description of Artificial Sequence/note = Synthetic Construct
gtattggcttcgcatcaggcagcagnnagcccattgattgcttrgtaaca

SEQ ID NO: 352 - DNA, 50 bp, Artificial Sequence
  Description of Artificial Sequence/note = Synthetic Construct
ataccctcttggtgtgtgtgtggcatcatcagtcttaacatccaaaccaa

SEQ ID NO: 353 - DNA, 50 bp, Artificial Sequence
  Description of Artificial Sequence/note = Synthetic Construct
gccctaaggcatccattgtatgtaatgaattaacttctctcctatgcatc

SEQ ID NO: 354 - DNA, 50 bp, Artificial Sequence
  Description of Artificial Sequence/note = Synthetic Construct
catctgtccagtgttgggtgtcatgtgtttarccatccccataaccctag

SEQ ID NO: 355 - DNA, 50 bp, Artificial Sequence
  Description of Artificial Sequence/note = Synthetic Construct
tataaagccaacctcctctgctcagctcatyggaacactcattctatttt

SEQ ID NO: 356 - DNA, 50 bp, Artificial Sequence
  Description of Artificial Sequence/note = Synthetic Construct
tgtggtattaaaatttcatggngggggggggtgattaggaaaaaaatgtc

SEQ ID NO: 357 - DNA, 50 bp, Artificial Sequence
  Description of Artificial Sequence/note = Synthetic Construct
ttctacttatcactagagacagaaactaaaaaccatggcttcaggctgct

SEQ ID NO: 358 - DNA, 50 bp, Artificial Sequence
  Description of Artificial Sequence/note = Synthetic Construct
acttaataatggccccaaagcgcaagagtagtgatgctggcatattgtta

SEQ ID NO: 359 - DNA, 50 bp, Artificial Sequence
  Description of Artificial Sequence/note = Synthetic Construct
ctactgacagcaggggagatagggcatacgtgggtagagcggataattcc

SEQ ID NO: 360 - DNA, 50 bp, Artificial Sequence
  Description of Artificial Sequence/note = Synthetic Construct
ccctggaaggctttcaggtcagcttcaacttactggccagagttgtgctg

SEQ ID NO: 361 - DNA, 50 bp, Artificial Sequence
  Description of Artificial Sequence/note = Synthetic Construct
cctctttgcagacagccccttctctgctgtgctgcccgttgcaaccttgc

SEQ ID NO: 362 - DNA, 50 bp, Artificial Sequence
  Description of Artificial Sequence/note = Synthetic Construct
gcacgtagccccctccagtacaaccctataaaacttccctccagcccctg

SEQ ID NO: 363 - DNA, 50 bp, Artificial Sequence
  Description of Artificial Sequence/note = Synthetic Construct
gaaagaacctgggtccttgatgatatcgttgagccgctgaattaaccaac

SEQ ID NO: 364 - DNA, 50 bp, Artificial Sequence
  Description of Artificial Sequence/note = Synthetic Construct
atcagacgcaragacaacagccttacagagactgcttaaccagctcccac

SEQ ID NO: 365 - DNA, 50 bp, Artificial Sequence
  Description of Artificial Sequence/note = Synthetic Construct
tcatatctttttccttgatcagcccccaaatcccttraacccccttcaca

SEQ ID NO: 366 - DNA, 50 bp, Artificial Sequence
  Description of Artificial Sequence/note = Synthetic Construct
ctcttttttgcctttaaaaatccacttgtaactgctgctaattggagtgt

SEQ ID NO: 367 - DNA, 50 bp, Artificial Sequence
  Description of Artificial Sequence/note = Synthetic Construct
gagtgccctgtatgtaagtcctaataaactcatctacttatcaagctgga

SEQ ID NO: 368 - DNA, 50 bp, Artificial Sequence
  Description of Artificial Sequence/note = Synthetic Construct
agccgcaagcctattaaaccttgcctgagaaaatcggtttggcctggtgt

SEQ ID NO: 369 - DNA, 50 bp, Artificial Sequence
  Description of Artificial Sequence/note = Synthetic Construct
atttttcctrgrtgtgccctcaagctggctcagtaaacctcgatgntttg

SEQ ID NO: 370 - DNA, 50 bp, Artificial Sequence
  Description of Artificial Sequence/note = Synthetic Construct
ctganaggataaagatacctcgtgacaaagcctcctgggtataatactcc

SEQ ID NO: 371 - DNA, 50 bp, Artificial Sequence
  Description of Artificial Sequence/note = Synthetic Construct
aaaatggcttccctgggttcttccctttttaggcccacttgttagtctcc

SEQ ID NO: 372 - DNA, 50 bp, Artificial Sequence
  Description of Artificial Sequence/note = Synthetic Construct
tccaattacaggtgtgacgttttcattcctcatcattatcccacaacgcc

SEQ ID NO: 373 - DNA, 50 bp, Artificial Sequence
  Description of Artificial Sequence/note = Synthetic Construct
tcggtgtattgacttgccgcgcatcgggcaacaaacctattacggtcaca

SEQ ID NO: 374 - DNA, 50 bp, Artificial Sequence
  Description of Artificial Sequence/note = Synthetic Construct
ctgccctatcctgcttccctcactcccttacaagtttctcctgagagcac

SEQ ID NO: 375 - DNA, 50 bp, Artificial Sequence
  Description of Artificial Sequence/note = Synthetic Construct
tctttggaatctgtgyttccngggtggnccatcntcaaactttgcacttg

SEQ ID NO: 376 - DNA, 50 bp, Artificial Sequence
  Description of Artificial Sequence/note = Synthetic Construct
cccgctcctgctccctccccttttatctttcacaggntttcccctaataa

SEQ ID NO: 377 - DNA, 50 bp, Artificial Sequence
  Description of Artificial Sequence/note = Synthetic Construct
cttcaaraaaaatcygacatcataaaaaccccgtgcagactctcagggct

SEQ ID NO: 378 - DNA, 50 bp, Artificial Sequence
  Description of Artificial Sequence/note = Synthetic Construct
gtaggcagaattctaagatggcccccaagattcccaccccctggtgtaca

SEQ ID NO: 379 - DNA, 50 bp, Artificial Sequence
  Description of Artificial Sequence/note = Synthetic Construct
tagccaacggaatgtaagcagaagtgatgtgcgccacttccaggcctggc

SEQ ID NO: 380 - DNA, 50 bp, Artificial Sequence
  Description of Artificial Sequence/note = Synthetic Construct
cctgagtcactacntggaggagagccacccacacccgaccagaacccnca

SEQ ID NO: 381 - DNA, 50 bp, Artificial Sequence
  Description of Artificial Sequence/note = Synthetic Construct
tcrgctrgggrcrgtcagagargagntcagccgctggayngccaaactcc

SEQ ID NO: 382 - DNA, 50 bp, Artificial Sequence
  Description of Artificial Sequence/note = Synthetic Construct
tgtccrtcattnctggcatngtcaggactaggtamggtctcgdccaactg

SEQ ID NO: 383 - DNA, 50 bp, Artificial Sequence
  Description of Artificial Sequence/note = Synthetic Construct
gccccccaaagatgtccatgccctaatccctggaacctgtgaatatgtta

SEQ ID NO: 384 - DNA, 50 bp, Artificial Sequence
  Description of Artificial Sequence/note = Synthetic Construct
cactggctggtcggcaactgtttacagcactctcctgggagtctgtaagc

SEQ ID NO: 385 - DNA, 50 bp, Artificial Sequence
  Description of Artificial Sequence/note = Synthetic Construct
tttccaaagatggccgcaacaatatctcccatcccacatgctcttcttac

SEQ ID NO: 386 - DNA, 50 bp, Artificial Sequence
  Description of Artificial Sequence/note = Synthetic Construct
gcccatttccaggcataaatactatttacctcagtctctactgttcttct

SEQ ID NO: 387 - DNA, 50 bp, Artificial Sequence
  Description of Artificial Sequence/note = Synthetic Construct
ctcgcctcactgtgcccaccaatccaaagctattatgtcataaactctgc

SEQ ID NO: 388 - DNA, 50 bp, Artificial Sequence
  Description of Artificial Sequence/note = Synthetic Construct
caaagaatcctgcgtcaaaatcgagagaacgaacaagccttcatcgccat

SEQ ID NO: 389 - DNA, 50 bp, Artificial Sequence
  Description of Artificial Sequence/note = Synthetic Construct
aataaaaaggctggacaagatatatggtggagggatgcacatacaaagag

SEQ ID NO: 390 - DNA, 50 bp, Artificial Sequence
  Description of Artificial Sequence/note = Synthetic Construct
caggcgtctccacggagtccaatgaaaaactcgaagccagcgacaagcaa

SEQ ID NO: 391 - DNA, 50 bp, Artificial Sequence
  Description of Artificial Sequence/note = Synthetic Construct
ctcatagctcctataatgccattgaacaccagtgagagacgattagacgt

SEQ ID NO: 392 - DNA, 50 bp, Artificial Sequence
  Description of Artificial Sequence/note = Synthetic Construct
accgccactgctacacatcttatcgaatgactcacgagttctccttcact

SEQ ID NO: 393 - DNA, 50 bp, Artificial Sequence
  Description of Artificial Sequence/note = Synthetic Construct
atccactgagctggtgcgtaccttaaaataaataacaatcctcctgtatt

SEQ ID NO: 394 - DNA, 50 bp, Artificial Sequence
  Description of Artificial Sequence/note = Synthetic Construct
ctcaatttgttttctcccctcctttgcctatctctatctaacaacctcta

SEQ ID NO: 395 - DNA, 50 bp, Artificial Sequence
  Description of Artificial Sequence/note = Synthetic Construct
atagaggcagtagtaacccgaaacactaccatgctattgacggcattaac

SEQ ID NO: 396 - DNA, 50 bp, Artificial Sequence
  Description of Artificial Sequence/note = Synthetic Construct
caaanatgtgtggacctggttatctctgaccttgcrctgctcacgacaca

SEQ ID NO: 397 - DNA, 50 bp, Artificial Sequence
  Description of Artificial Sequence/note = Synthetic Construct
ggctataggcntycctcagtctacagtcctcagtaagacttctgaataaa

SEQ ID NO: 398 - DNA, 50 bp, Artificial Sequence
  Description of Artificial Sequence/note = Synthetic Construct
ccagaccagtggctttcaaactttttttgactatgacccacagtaagaaa

SEQ ID NO: 399 - DNA, 50 bp, Artificial Sequence
  Description of Artificial Sequence/note = Synthetic Construct
aagcaccaaactgagactttctccttgatgtaatcagaaggattgaaaga

SEQ ID NO: 400 - DNA, 50 bp, Artificial Sequence
  Description of Artificial Sequence/note = Synthetic Construct
ttacccaatcctaatcaagcccctacattgaaagacctgccttaaatcag

SEQ ID NO: 401 - DNA, 50 bp, Artificial Sequence
  Description of Artificial Sequence/note = Synthetic Construct
cttcttgctgttgctaatctctgggttgcctcaccattgnttccctgttt

SEQ ID NO: 402 - DNA, 50 bp, Artificial Sequence
  Description of Artificial Sequence/note = Synthetic Construct
ccccggccgacatcttgactgcaacctcatgagagaccctgagccagaac

SEQ ID NO: 403 - DNA, 50 bp, Artificial Sequence
  Description of Artificial Sequence/note = Synthetic Construct
acaccccccccstacvcccacmccccctgtgatattgttcgtaatatcca

SEQ ID NO: 404 - DNA, 50 bp, Artificial Sequence
  Description of Artificial Sequence/note = Synthetic Construct
tttaaatatttagacatatggtatgtgggcctccatttgtactcttgccc

SEQ ID NO: 405 - DNA, 50 bp, Artificial Sequence
  Description of Artificial Sequence/note = Synthetic Construct
gcacaggagggggaagtagcagcanatatgctatgtatttgccatccctg

SEQ ID NO: 406 - DNA, 50 bp, Artificial Sequence
  Description of Artificial Sequence/note = Synthetic Construct
taggtgcaagcatctgactacttcattatgtcttctagtgtagtcatgcc

SEQ ID NO: 407 - DNA, 50 bp, Artificial Sequence
  Description of Artificial Sequence/note = Synthetic Construct
tccatggttcctctggtgtgcagtctccctcattgcaataagtcaataaa

SEQ ID NO: 408 - DNA, 50 bp, Artificial Sequence
  Description of Artificial Sequence/note = Synthetic Construct
tgaagyggttgctttggataggaatcyggccrcttccccattactagttt

SEQ ID NO: 409 - DNA, 50 bp, Artificial Sequence
  Description of Artificial Sequence/note = Synthetic Construct
ggattgacagcagatcamgggaagtgattatacccctttacaatgccttg

SEQ ID NO: 410 - DNA, 50 bp, Artificial Sequence
  Description of Artificial Sequence/note = Synthetic Construct
gtgggatggacagggatgggagggactgacttttcactgtataccttttt

SEQ ID NO: 411 - DNA, 50 bp, Artificial Sequence
  Description of Artificial Sequence/note = Synthetic Construct
tggaccctccagaccagcccatctgccagctgaataccactgagtgacct

SEQ ID NO: 412 - DNA, 50 bp, Artificial Sequence
  Description of Artificial Sequence/note = Synthetic Construct
gggacagaaattgtgcactcggggagctcggattttaaggcagtagcttg

SEQ ID NO: 413 - DNA, 50 bp, Artificial Sequence
  Description of Artificial Sequence/note = Synthetic Construct
ccagaaaccacctccccacaagcccactagaaacaaacatctgacagaga

SEQ ID NO: 414 - DNA, 50 bp, Artificial Sequence
  Description of Artificial Sequence/note = Synthetic Construct
tagccnataaaatactcttaacagctccagnaacagttgcatcagcagaa

SEQ ID NO: 415 - DNA, 50 bp, Artificial Sequence
  Description of Artificial Sequence/note = Synthetic Construct
tttaaaacatggccgcaaattctttgacactcctctcattgagangtggg

SEQ ID NO: 416 - DNA, 50 bp, Artificial Sequence
  Description of Artificial Sequence/note = Synthetic Construct
cttgcttcctctctcaccatgtgatctctgcacacgctggctccccttcc

SEQ ID NO: 417 - DNA, 50 bp, Artificial Sequence
  Description of Artificial Sequence/note = Synthetic Construct
gaattcgtctcaaagtgtggcgtttctctataactcgctcggttacaaca

SEQ ID NO: 418 - DNA, 50 bp, Artificial Sequence
  Description of Artificial Sequence/note = Synthetic Construct
ggtctggaaaccatgtcatatgaggaacggttgaaggaactggggatgtt

SEQ ID NO: 419 - DNA, 50 bp, Artificial Sequence
  Description of Artificial Sequence/note = Synthetic Construct
tgccatttacgtgggataaagcttgtttacccttaaaggtattgtgtgtg

SEQ ID NO: 420 - DNA, 50 bp, Artificial Sequence
  Description of Artificial Sequence/note = Synthetic Construct
accttttgtcggaactcggagttatgaacgaccctcaccataccgatgct

SEQ ID NO: 421 - DNA, 50 bp, Artificial Sequence
  Description of Artificial Sequence/note = Synthetic Construct
tatngcctcccaaggtgactactttgaaggggacaacactcatttggatg

SEQ ID NO: 422 - DNA, 50 bp, Artificial Sequence
  Description of Artificial Sequence/note = Synthetic Construct
ttactgagacactaagggcgccgtgaaccgagaaagtttgggaacctctg

SEQ ID NO: 423 - DNA, 50 bp, Artificial Sequence
  Description of Artificial Sequence/note = Synthetic Construct
gttctccagccctcccggagattctgtgagctacccaatatcctttaata

SEQ ID NO: 424 - DNA, 50 bp, Artificial Sequence
  Description of Artificial Sequence/note = Synthetic Construct
cgggcngattggtgagatccntctcctacacgaggccagtctgacaagac

SEQ ID NO: 425 - DNA, 50 bp, Artificial Sequence
  Description of Artificial Sequence/note = Synthetic Construct
ctcttatggactatctccgtgcaattgcccataatctatccctgtaatat

SEQ ID NO: 426 - DNA, 50 bp, Artificial Sequence
  Description of Artificial Sequence/note = Synthetic Construct
aggggtctggggagtcatgccctacaaaccataaattctcatcagatggg

SEQ ID NO: 427 - DNA, 50 bp, Artificial Sequence
  Description of Artificial Sequence/note = Synthetic Construct
acctttcgcgtttcagttaacaaaccatttaaggaccatttgaggaagga

SEQ ID NO: 428 - DNA, 50 bp, Artificial Sequence
  Description of Artificial Sequence/note = Synthetic Construct
tgctcatgctgcttgctgtgycatgagtaataaagtcctttgtctctgac

SEQ ID NO: 429 - DNA, 50 bp, Artificial Sequence
  Description of Artificial Sequence/note = Synthetic Construct
tgctcaagctactttacaaaagccaaactgctctgccatgcccagcggag

SEQ ID NO: 430 - DNA, 50 bp, Artificial Sequence
  Description of Artificial Sequence/note = Synthetic Construct
ggaagcagtatggtatagtggaaagaacaactggactaggagtcaggaga

SEQ ID NO: 431 - DNA, 50 bp, Artificial Sequence
  Description of Artificial Sequence/note = Synthetic Construct
ccagctgtcaagtcatccccagcctctnncagycmtccccagccttcaag

SEQ ID NO: 432 - DNA, 50 bp, Artificial Sequence
  Description of Artificial Sequence/note = Synthetic Construct
ccacttcccctttgaccttctctgccatgttatgatgcagcatgaaagcc

SEQ ID NO: 433 - DNA, 50 bp, Artificial Sequence
  Description of Artificial Sequence/note = Synthetic Construct
tttgagaactgaactaaaggatagaccactacccaggtcccagactggcc

SEQ ID NO: 434 - DNA, 50 bp, Artificial Sequence
  Description of Artificial Sequence/note = Synthetic Construct
artgctaatttttctttgcagcaccgaggaacaagcattctgtttctaaa

SEQ ID NO: 435 - DNA, 50 bp, Artificial Sequence
  Description of Artificial Sequence/note = Synthetic Construct
tctctggagtctgtgtttcctgaatggccattcccagcttttnacttgaa

SEQ ID NO: 436 - DNA, 50 bp, Artificial Sequence
  Description of Artificial Sequence/note = Synthetic Construct
tggagtgatgcagccataagccaaggaatgccagcagccaagccaccaga

SEQ ID NO: 437 - DNA, 50 bp, Artificial Sequence
  Description of Artificial Sequence/note = Synthetic Construct
gtgggtttgttataaaagnaagttcggcccccttttgctctctcnctctc

SEQ ID NO: 438 - DNA, 50 bp, Artificial Sequence
  Description of Artificial Sequence/note = Synthetic Construct
atctttacgtcatatacatttccatgtctcaggaggctagggctttttac

SEQ ID NO: 439 - DNA, 50 bp, Artificial Sequence
  Description of Artificial Sequence/note = Synthetic Construct
taaaaacccagtggataggtnaaacagcagattaganacagctgaagaga

SEQ ID NO: 440 - DNA, 50 bp, Artificial Sequence
  Description of Artificial Sequence/note = Synthetic Construct
actgaaaggaaatatacaccaaaatgttaacagtggttatctctgggtgg

SEQ ID NO: 441 - DNA, 50 bp, Artificial Sequence
  Description of Artificial Sequence/note = Synthetic Construct
tagaagaaatagctgaccgtgggaatgttgacactgccgccatttgagag

SEQ ID NO: 442 - DNA, 50 bp, Artificial Sequence
  Description of Artificial Sequence/note = Synthetic Construct
agaccaaatccttcatccagataaggggtagccaataggaacctcaaaag

SEQ ID NO: 443 - DNA, 50 bp, Artificial Sequence
  Description of Artificial Sequence/note = Synthetic Construct
ccggctaaataaacggactcttaattcgtctcaaagtgtggcgttttctc

SEQ ID NO: 444 - DNA, 50 bp, Artificial Sequence
  Description of Artificial Sequence/note = Synthetic Construct
tccacagttcctggctcataactcccatagcccttgttacagtcttttgt

SEQ ID NO: 445 - DNA, 50 bp, Artificial Sequence
  Description of Artificial Sequence/note = Synthetic Construct
ccacaagttgctgcccctagagactcaaagtccttttcctttgtcttgtc

SEQ ID NO: 446 - DNA, 50 bp, Artificial Sequence
  Description of Artificial Sequence/note = Synthetic Construct
agtttcttttgtcttaagttttcatttctgcgttcgtcccccttcgttca

SEQ ID NO: 447 - DNA, 50 bp, Artificial Sequence
  Description of Artificial Sequence/note = Synthetic Construct
aggcggttgtataaggcagatatctggatcgaccacattgaggaactggg

SEQ ID NO: 448 - DNA, 50 bp, Artificial Sequence
  Description of Artificial Sequence/note = Synthetic Construct
gcctttcatctatccgagtgtcantgtgttgtgtcccgccatcaaaagaa

SEQ ID NO: 449 - DNA, 50 bp, Artificial Sequence
  Description of Artificial Sequence/note = Synthetic Construct
aagagtaaacatcactcaaggactttacctcctcttctggggaaggggtt

SEQ ID NO: 450 - DNA, 50 bp, Artificial Sequence
  Description of Artificial Sequence/note = Synthetic Construct
aaataccccnaataattgatgtcaaaactgacgtcaagacanaaaggggt

SEQ ID NO: 451 - DNA, 50 bp, Artificial Sequence
  Description of Artificial Sequence/note = Synthetic Construct
taagtcccaactcagggatttaggtccacgtaacctcctgaccgactaac

SEQ ID NO: 452 - DNA, 50 bp, Artificial Sequence
  Description of Artificial Sequence/note = Synthetic Construct
tctccgatgagttctttcctccagcaagatccaatatcctaagtcccaca

SEQ ID NO: 453 - DNA, 50 bp, Artificial Sequence
  Description of Artificial Sequence/note = Synthetic Construct
attttccctttcttgagaccccaataggcagcaggtagacatgagcatgg

SEQ ID NO: 454 - DNA, 50 bp, Artificial Sequence
  Description of Artificial Sequence/note = Synthetic Construct
taataaactgtctgaatctaaaagtggctcgttgtatctttaccagccga

SEQ ID NO: 455 - DNA, 50 bp, Artificial Sequence
  Description of Artificial Sequence/note = Synthetic Construct
caccgagctagctgcaggagtttttttttttcgtaccccagtggcgcctg

SEQ ID NO: 456 - DNA, 50 bp, Artificial Sequence
  Description of Artificial Sequence/note = Synthetic Construct
ctttagccctaggggaactgtcggacctgaactctgcagggcggtcttgc

SEQ ID NO: 457 - DNA, 50 bp, Artificial Sequence
  Description of Artificial Sequence/note = Synthetic Construct
aagaaacaaataacatacaatggagctccaatacgtctggcagcagactt

SEQ ID NO: 458 - DNA, 50 bp, Artificial Sequence
  Description of Artificial Sequence/note = Synthetic Construct
catgtcagacccgacaccaagagggatcccctcggctaagtctccccatt

SEQ ID NO: 459 - DNA, 50 bp, Artificial Sequence
  Description of Artificial Sequence/note = Synthetic Construct
cccattcgggacgggcagcgctctgattgtttactagagccgaggcaaac

SEQ ID NO: 460 - DNA, 50 bp, Artificial Sequence
  Description of Artificial Sequence/note = Synthetic Construct
aaaggggtggggatggagctgtaaaggagcagagtttttgtatgttattg

SEQ ID NO: 461 - DNA, 50 bp, Artificial Sequence
  Description of Artificial Sequence/note = Synthetic Construct
cacaaaagtaggccaggacctgcatgctaaacctaaacagggtgactgcc

SEQ ID NO: 462 - DNA, 50 bp, Artificial Sequence
  Description of Artificial Sequence/note = Synthetic Construct
cacaggaaggggaatatcacactctggggactgtggtggggtcgggggag

SEQ ID NO: 463 - DNA, 50 bp, Artificial Sequence
  Description of Artificial Sequence/note = Synthetic Construct
aacacatggacacaggaaggggaacatcacactctggggactgttgtggg

SEQ ID NO: 464 - DNA, 50 bp, Artificial Sequence
  Description of Artificial Sequence/note = Synthetic Construct
aacacatggacacaggaaggggaacatcacacaccggggcctgttgtggg

SEQ ID NO: 465 - DNA, 50 bp, Artificial Sequence
  Description of Artificial Sequence/note = Synthetic Construct
gaacacttggacacaggaaggggaacatcacacaccggggcctgttgtgg

SEQ ID NO: 466 - DNA, 50 bp, Artificial Sequence
  Description of Artificial Sequence/note = Synthetic Construct
gagaaatacctaatgtaaatgacgagttgatgggtgcagcaaaccaacat

SEQ ID NO: 467 - DNA, 50 bp, Artificial Sequence
  Description of Artificial Sequence/note = Synthetic Construct
aggacaaatacctaatgcatgcggggcttaaaacctagatgacgggttga

SEQ ID NO: 468 - DNA, 50 bp, Artificial Sequence
  Description of Artificial Sequence/note = Synthetic Construct
atagctaatgcatgctgggcttaatacctaggtgatgggttgataggtgc

SEQ ID NO: 469 - DNA, 50 bp, Artificial Sequence
  Description of Artificial Sequence/note = Synthetic Construct
cttaatacctgggtgatgaaataatctgtacaacaaacccccatgacaca

SEQ ID NO: 470 - DNA, 50 bp, Artificial Sequence
  Description of Artificial Sequence/note = Synthetic Construct
tacctgggtgatgaaataatctgtacaacaaacccccatgacacaagttt

SEQ ID NO: 471 - DNA, 50 bp, Artificial Sequence
  Description of Artificial Sequence/note = Synthetic Construct
gggagaggagcagaaaagataactattgggtactgggcttaatacctggg

SEQ ID NO: 472 - DNA, 50 bp, Artificial Sequence
  Description of Artificial Sequence/note = Synthetic Construct
tgggtgatgggatcattcgtaccccaaacctcagcatcacgcaatatacc

SEQ ID NO: 473 - DNA, 50 bp, Artificial Sequence
  Description of Artificial Sequence/note = Synthetic Construct
atctcagaaatcaccactaaagaacttatccatgtaaccaaaaaccacct

SEQ ID NO: 474 - DNA, 50 bp, Artificial Sequence
  Description of Artificial Sequence/note = Synthetic Construct
ktacactaaaagcccagacttcaccactacgcaatatatccatgtaacaa

SEQ ID NO: 475 - DNA, 50 bp, Artificial Sequence
  Description of Artificial Sequence/note = Synthetic Construct
attctccatgatgtgcttatttcacattgcatgcctgtatcaaaacatct

SEQ ID NO: 476 - DNA, 50 bp, Artificial Sequence
  Description of Artificial Sequence/note = Synthetic Construct
gctgggaagggtagtggggtgggggggaagtggggatggttaatgggtac

SEQ ID NO: 477 - DNA, 50 bp, Artificial Sequence
  Description of Artificial Sequence/note = Synthetic Construct
ggagggggggaatgaagagaggttggttaatgggtacaaaaatacagtta

SEQ ID NO: 478 - DNA, 50 bp, Artificial Sequence
  Description of Artificial Sequence/note = Synthetic Construct
gaggacttgaaatgttcccaacacatagaaatgataaatactcgaggtga

SEQ ID NO: 479 - DNA, 50 bp, Artificial Sequence
  Description of Artificial Sequence/note = Synthetic Construct
tgggaagggtagggggaagggggggatagggagagatttgttaaaggata

SEQ ID NO: 480 - DNA, 50 bp, Artificial Sequence
  Description of Artificial Sequence/note = Synthetic Construct
ataggaggaataagttctggtgttctattgcacagtagggtgactatagt

SEQ ID NO: 481 - DNA, 50 bp, Artificial Sequence
  Description of Artificial Sequence/note = Synthetic Construct
atggggagatgttggtcaaagggtacaaagtttcagttagacaggaggaa

SEQ ID NO: 482 - DNA, 50 bp, Artificial Sequence
  Description of Artificial Sequence/note = Synthetic Construct
tgctnatggtcccatgactggccactctgtgaacacagtaaacaagtttg

SEQ ID NO: 483 - DNA, 50 bp, Artificial Sequence
  Description of Artificial Sequence/note = Synthetic Construct
gaaatggggagttgctgttcaatgggtataaagtttcagttatgcaagat

SEQ ID NO: 484 - DNA, 50 bp, Artificial Sequence
  Description of Artificial Sequence/note = Synthetic Construct
gggtatagagtttcagttttgcaagatgaaaaagttctggagatcggttg

SEQ ID NO: 485 - DNA, 50 bp, Artificial Sequence
  Description of Artificial Sequence/note = Synthetic Construct
tggtgatggttgcacaacamtgtgaatgtacttaatgccactgaattgta

SEQ ID NO: 486 - DNA, 50 bp, Artificial Sequence
  Description of Artificial Sequence/note = Synthetic Construct
agggggaatggggagtgactgcttaatgggtacggggtttccttttgggg

SEQ ID NO: 487 - DNA, 50 bp, Artificial Sequence
  Description of Artificial Sequence/note = Synthetic Construct
ggaatggggagtgactgctaatgggtacggggtttcttttgggggtgatg

SEQ ID NO: 488 - DNA, 50 bp, Artificial Sequence
  Description of Artificial Sequence/note = Synthetic Construct
ggtgggggnaggggattgactacaaaggggcatgagggaactttttgggg

SEQ ID NO: 489 - DNA, 50 bp, Artificial Sequence
  Description of Artificial Sequence/note = Synthetic Construct
atagtggttacctttggggagggttattgactgggaaggggcatgaggga

SEQ ID NO: 490 - DNA, 50 bp, Artificial Sequence
  Description of Artificial Sequence/note = Synthetic Construct
gactggaaggaaatacaccaaaatgttaacagtggttatctctgggtggt

SEQ ID NO: 491 - DNA, 50 bp, Artificial Sequence
  Description of Artificial Sequence/note = Synthetic Construct
ttgatagtgggggaggctgtgcatgtgtgggggcagggggtatatgggaa

SEQ ID NO: 492 - DNA, 50 bp, Artificial Sequence
  Description of Artificial Sequence/note = Synthetic Construct
acccataaccccagtctaatcatgagaaaacatcagacaaacccaaattg

SEQ ID NO: 493 - DNA, 50 bp, Artificial Sequence
  Description of Artificial Sequence/note = Synthetic Construct
agaggagaggtggaaggaagtatgagagtgctaatntcctcatctttcat

SEQ ID NO: 494 - DNA, 50 bp, Artificial Sequence
  Description of Artificial Sequence/note = Synthetic Construct
agacccagggttcaggcctgtcccagtagaccccagcactaggctagtcc

SEQ ID NO: 495 - DNA, 50 bp, Artificial Sequence
  Description of Artificial Sequence/note = Synthetic Construct
aagaaggaatcttggaacatcaggaaggaagaaagaacatagtaagaagc

SEQ ID NO: 496 - DNA, 50 bp, Artificial Sequence
  Description of Artificial Sequence/note = Synthetic Construct
ggcagaaactggaggggagtcgacacctggaagaagggaatwgcacggag

SEQ ID NO: 497 - DNA, 50 bp, Artificial Sequence; identical to SEQ ID NO: 294
  Description of Artificial Sequence/note = Synthetic Construct

SEQ ID NO: 498 - DNA, 50 bp, Artificial Sequence
  Description of Artificial Sequence/note = Synthetic Construct
aggcaaccccatcaagaacttangcgaaaaaagatgtaggatcacaaagt

SEQ ID NO: 499 - DNA, 50 bp, Artificial Sequence
  Description of Artificial Sequence/note = Synthetic Construct
tcggatggaacgcagcattaaagtcacccatatgatcaatgaaggattac

SEQ ID NO: 500 - DNA, 50 bp, Artificial Sequence
  Description of Artificial Sequence/note = Synthetic Construct
cctcacttcatctcatcacgtaggcattttatcatctcacatcatcacaa

SEQ ID NO: 501 - DNA, 50 bp, Artificial Sequence
  Description of Artificial Sequence/note = Synthetic Construct
atcgacgaagataacataaaactcataatacgccactacaacgaggacat

SEQ ID NO: 502 - DNA, 50 bp, Artificial Sequence
  Description of Artificial Sequence/note = Synthetic Construct
tatttatgtttgatcctcagtgctttgtgtgacttgggctttgagaatta

SEQ ID NO: 503 - DNA, 50 bp, Artificial Sequence
  Description of Artificial Sequence/note = Synthetic Construct
gattggtttgacaatgaggactggctttgccaattaggttatatggcaga

SEQ ID NO: 504 - DNA, 50 bp, Artificial Sequence
  Description of Artificial Sequence/note = Synthetic Construct
ttaatncaccttttgtaagccctatacttactagtggcccaataccttct

SEQ ID NO: 505 - DNA, 50 bp, Artificial Sequence
  Description of Artificial Sequence/note = Synthetic Construct
acttagaaccagaccttcgaatcgctgtatcacaaagtgttaaaccaaga

SEQ ID NO: 506 - DNA, 50 bp, Artificial Sequence
  Description of Artificial Sequence/note = Synthetic Construct
atttatgttacctgcctggcccctgtaggcatttgagtttgcgacccctg

SEQ ID NO: 507 - DNA, 50 bp, Artificial Sequence; identical to SEQ ID NO: 506
  Description of Artificial Sequence/note = Synthetic Construct

SEQ ID NO: 508 - DNA, 50 bp, Artificial Sequence; identical to SEQ ID NO: 158
  Description of Artificial Sequence/note = Synthetic Construct

SEQ ID NO: 509 - DNA, 50 bp, Artificial Sequence
  Description of Artificial Sequence/note = Synthetic Construct
tgttaaaaaatgatccgctctgggtgtcgaatacgctaggtacgccactg

SEQ ID NO: 510 - DNA, 50 bp, Artificial Sequence
  Description of Artificial Sequence/note = Synthetic Construct
ccagtggtatgntttwgtagttgcctaaattgtaccttttgcagacgttt

SEQ ID NO: 511 - DNA, 50 bp, Artificial Sequence; identical to SEQ ID NO: 509
  Description of Artificial Sequence/note = Synthetic Construct

SEQ ID NO: 512 - DNA, 50 bp, Artificial Sequence
  Description of Artificial Sequence/note = Synthetic Construct
gttcttggaaactgcgactttaagcgaaacgacgtacagcaggtcctcga

SEQ ID NO: 513 - DNA, 50 bp, Artificial Sequence
  Description of Artificial Sequence/note = Synthetic Construct
attgccggcccatcaacagaacacccagacatgtgcaataataattaaat

SEQ ID NO: 514 - DNA, 50 bp, Artificial Sequence
  Description of Artificial Sequence/note = Synthetic Construct
gccagtcagatttcacggcantgccaatgtttctgtctgtacagcgntgt

SEQ ID NO: 515 - DNA, 50 bp, Artificial Sequence
  Description of Artificial Sequence/note = Synthetic Construct
ctcctgtgcttaccctgtatctgtaatctatatcaactatgccttcccca

SEQ ID NO: 516 - DNA, 50 bp, Artificial Sequence
  Description of Artificial Sequence/note = Synthetic Construct
tttatcaggggtttccgcttttgcttcttcctcattttcctcttgccgcc

SEQ ID NO: 517 - DNA, 50 bp, Artificial Sequence
  Description of Artificial Sequence/note = Synthetic Construct
gtgtccccacccaaatctcatcttgaattgtagttcccataatccccacg

SEQ ID NO: 518 - DNA, 50 bp, Artificial Sequence
  Description of Artificial Sequence/note = Synthetic Construct
tgttagttcacgcgagatctggttgtttaaaagagtntggcacctccccc

SEQ ID NO: 519 - DNA, 50 bp, Artificial Sequence
  Description of Artificial Sequence/note = Synthetic Construct
cttcctctctcgccatgtgatctctgcacacgccggctccccttcacctt

SEQ ID NO: 520 - DNA, 50 bp, Artificial Sequence
  Description of Artificial Sequence/note = Synthetic Construct
tcagtctgctccctatcttcggctgcccgtttagntgtggctcaagtggg

SEQ ID NO: 521 - DNA, 50 bp, Artificial Sequence
  Description of Artificial Sequence/note = Synthetic Construct
aaggtgcggcctggtttctccttgctgcttatagtaaaatgcgagaggaa

SEQ ID NO: 522 - DNA, 50 bp, Artificial Sequence
  Description of Artificial Sequence/note = Synthetic Construct
cctttcgcgtttcagttaacaaaccatttaaggaccatttgaggaaggaa

SEQ ID NO: 523 - DNA, 50 bp, Artificial Sequence
  Description of Artificial Sequence/note = Synthetic Construct
tgaaggcaggagaaattgccnaatcccncggaatagatgaaagaaatttc

SEQ ID NO: 524 - DNA, 50 bp, Artificial Sequence
  Description of Artificial Sequence/note = Synthetic Construct
tnatgtagactccttcgcaagactccatcagcgaaccatttgacactttt

SEQ ID NO: 525 - DNA, 50 bp, Artificial Sequence
  Description of Artificial Sequence/note = Synthetic Construct
acgctcttcccccagatatccacgtggctsgctccytcacctcmttcagg

SEQ ID NO: 526 - DNA, 50 bp, Artificial Sequence
  Description of Artificial Sequence/note = Synthetic Construct
cctgccactctgggttatmattgtctgtkngcangtctgtctcccccact

SEQ ID NO: 527 - DNA, 50 bp, Artificial Sequence
  Description of Artificial Sequence/note = Synthetic Construct
tttgtttgggacaccaagagcctggaactgcacrgcaccakctggtaaca

SEQ ID NO: 528 - DNA, 50 bp, Artificial Sequence
  Description of Artificial Sequence/note = Synthetic Construct
tggaccagtgctagtctgcaaactgtttgttaccagtccatgataagata

SEQ ID NO: 529 - DNA, 50 bp, Artificial Sequence
  Description of Artificial Sequence/note = Synthetic Construct
cccggtgctgaagttttagacggtatctctgaggggttatctaatctcaa

SEQ ID NO: 530 - DNA, 50 bp, Artificial Sequence
  Description of Artificial Sequence/note = Synthetic Construct
gaaaagtcgcccctggggaagctggttaactaggaccacccaagaccccc

SEQ ID NO: 531 - DNA, 50 bp, Artificial Sequence
  Description of Artificial Sequence/note = Synthetic Construct
aaaaaaggagcttgaacactcagaaccctgaaatatgtttaaccaatgga

SEQ ID NO: 532 - DNA, 50 bp, Artificial Sequence
  Description of Artificial Sequence/note = Synthetic Construct
catagcaggaataatggttactaacagaaaataacacatgggcctttcca

SEQ ID NO: 533 - DNA, 50 bp, Artificial Sequence
  Description of Artificial Sequence/note = Synthetic Construct
tcactctgtgtgtgtgtgtccgcgacctcgatctccttggccgtgagacc

SEQ ID NO: 534 - DNA, 50 bp, Artificial Sequence
  Description of Artificial Sequence/note = Synthetic Construct
acccactgcttcaaaacccaaaccctgattacagcncccctattcggcag

SEQ ID NO: 535 - DNA, 50 bp, Artificial Sequence
  Description of Artificial Sequence/note = Synthetic Construct
tnaataagacatggcacatttcagtcatccatcaaacatcaggggtgaat

SEQ ID NO: 536 - DNA, 50 bp, Artificial Sequence
  Description of Artificial Sequence/note = Synthetic Construct
gcttctgcgcagccgctctctcatcagatgatcgccatgatgatacaaca

SEQ ID NO: 537 - DNA, 50 bp, Artificial Sequence
  Description of Artificial Sequence/note = Synthetic Construct
gacaatggtctntccttcagntcgggntgaagaatgaccaaaggagaaat

SEQ ID NO: 538 - DNA, 50 bp, Artificial Sequence
  Description of Artificial Sequence/note = Synthetic Construct
atccttgtttcgntgtaagggattcagtggttggaaancagggagtggcc

SEQ ID NO: 539 - DNA, 50 bp, Artificial Sequence
  Description of Artificial Sequence/note = Synthetic Construct
gcgctcaaagggtgagttaactggatcgtatgccgggagcctattgtttt

SEQ ID NO: 540 - DNA, 50 bp, Artificial Sequence
  Description of Artificial Sequence/note = Synthetic Construct
ctcgcggtcctggccatccttgnaggcatgggcataacgttatgttgtgg

SEQ ID NO: 541 - DNA, 50 bp, Artificial Sequence
  Description of Artificial Sequence/note = Synthetic Construct
acncccangggattatctactcccctaaacagctatctctcttctaaagt

SEQ ID NO: 542 - DNA, 50 bp, Artificial Sequence
  Description of Artificial Sequence/note = Synthetic Construct
agccatggctatacgttatagacctgtatagttcttcccctcatacccta

SEQ ID NO: 543 - DNA, 50 bp, Artificial Sequence
  Description of Artificial Sequence/note = Synthetic Construct
gggcatatgaaatggactagctttgctaagggggatatctgggttggggg

SEQ ID NO: 544 - DNA, 50 bp, Artificial Sequence
  Description of Artificial Sequence/note = Synthetic Construct
cgggatcggtttggagtgctccgtctgcatcggatccgtctgtgtttgtg

SEQ ID NO: 545 - DNA, 50 bp, Artificial Sequence
  Description of Artificial Sequence/note = Synthetic Construct
ctttccctacccactgccactacnyctgactctggggccaaagcacatgc

SEQ ID NO: 546 - DNA, 50 bp, Artificial Sequence
  Description of Artificial Sequence/note = Synthetic Construct
acaccccaatgaactgacaccaagacccatttatacaaataagtttttcc

SEQ ID NO: 547 - DNA, 50 bp, Artificial Sequence
  Description of Artificial Sequence/note = Synthetic Construct
ctggagcagtcctccaaaatagacggggattagatcttataacggctgaa

SEQ ID NO: 548 - DNA, 50 bp, Artificial Sequence
  Description of Artificial Sequence/note = Synthetic Construct
ctcagtggcagatggtagaggtcaagagagganggacactagcaaccagg

SEQ ID NO: 549 - DNA, 50 bp, Artificial Sequence
  Description of Artificial Sequence/note = Synthetic Construct
tctttgctcccaggttayaatcctnaagcttgrcccaaataaactgtcta

SEQ ID NO: 550 - DNA, 50 bp, Artificial Sequence
  Description of Artificial Sequence/note = Synthetic Construct
agatgtggatactcaagatttctattggggaaaactgtggtccttagtaa

SEQ ID NO: 551 - DNA, 50 bp, Artificial Sequence
  Description of Artificial Sequence/note = Synthetic Construct
tgtattgctggcagcagtgaggtgggttaagggtgctatccggggctgca

SEQ ID NO: 552 - DNA, 50 bp, Artificial Sequence
  Description of Artificial Sequence/note = Synthetic Construct
ttaaaagtctcgcttccactgttcttcgtgtctctgagtccattctttgg

SEQ ID NO: 553 - DNA, 50 bp, Artificial Sequence
  Description of Artificial Sequence/note = Synthetic Construct
cattaaaagtctcactttcgctgttctccgggtctctgagtccattcttt

SEQ ID NO: 554 - DNA, 50 bp, Artificial Sequence
  Description of Artificial Sequence/note = Synthetic Construct
cccaccagaaggaagaaactccggacacatctgaacatctgaaggaacaa

SEQ ID NO: 555 - DNA, 50 bp, Artificial Sequence
  Description of Artificial Sequence/note = Synthetic Construct
agcacttttttccccccttaatttttaaacccatgtgtatttcaagggaa

SEQ ID NO: 556 - DNA, 50 bp, Artificial Sequence
  Description of Artificial Sequence/note = Synthetic Construct
tgcagttggtggcgacagagactgtagtgtggctggagtggtaggaaggg

SEQ ID NO: 557 - DNA, 50 bp, Artificial Sequence
  Description of Artificial Sequence/note = Synthetic Construct
aaagctttattgctcacacaaagcctgtttggtggtctcttcacacggac

SEQ ID NO: 558 - DNA, 50 bp, Artificial Sequence
  Description of Artificial Sequence/note = Synthetic Construct
acagccttgttgctcacacaaagcctgtttggtggtctcttcacacggac

SEQ ID NO: 559 - DNA, 50 bp, Artificial Sequence
  Description of Artificial Sequence/note = Synthetic Construct
gattaggcagcayacaggccacatcctcactcctgtgataacaagacaga

SEQ ID NO: 560 - DNA, 50 bp, Artificial Sequence
  Description of Artificial Sequence/note = Synthetic Construct
caggagaatagaaaattccaggcagcagtttcacatgactagcaaaagga

SEQ ID NO: 561 - DNA, 50 bp, Artificial Sequence
  Description of Artificial Sequence/note = Synthetic Construct
aagataaatagccagacaaccttggcaccaccaccyggccctaggagtta

SEQ ID NO: 562 - DNA, 50 bp, Artificial Sequence
  Description of Artificial Sequence/note = Synthetic Construct
acacctcactcttgttattttggcttctttctacaagcggcaagcagcyg

SEQ ID NO: 563 - DNA, 50 bp, Artificial Sequence
  Description of Artificial Sequence/note = Synthetic Construct
aacctgtattctcatggagagtcgtttgttactcaccaggygaatraacc

SEQ ID NO: 564 - DNA, 50 bp, Artificial Sequence
  Description of Artificial Sequence/note = Synthetic Construct
taaaagcttccctttaccctcccctcttcagatgcatctgtggcttgcca

SEQ ID NO: 565 - DNA, 50 bp, Artificial Sequence
  Description of Artificial Sequence/note = Synthetic Construct
tgaggccttcgttggaaacgggatttcttcatataatgctagacagaaga

SEQ ID NO: 566 - DNA, 50 bp, Artificial Sequence
  Description of Artificial Sequence/note = Synthetic Construct
ggttccagcattcattcgctccggttcccgcactcactcgcttgcatgct

SEQ ID NO: 567 - DNA, 50 bp, Artificial Sequence
  Description of Artificial Sequence/note = Synthetic Construct
tctcacaagcagagggagtttcagcatttcagcaagttgtttcttttctt

SEQ ID NO: 568 - DNA, 50 bp, Artificial Sequence
  Description of Artificial Sequence/note = Synthetic Construct
gatgttaagtctgctgggtctgagtgcactcaataaaagatcctcctgtt

SEQ ID NO: 569 - DNA, 50 bp, Artificial Sequence
  Description of Artificial Sequence/note = Synthetic Construct
tttcacaatgcatcccttcctaaaaactgaccaccatctctggactggtt

SEQ ID NO: 570 - DNA, 50 bp, Artificial Sequence
  Description of Artificial Sequence/note = Synthetic Construct
gtgaagggatatttgggagctcattgaggcctatggtgaaaaagaaaata

SEQ ID NO: 571 - DNA, 50 bp, Artificial Sequence
  Description of Artificial Sequence/note = Synthetic Construct
gttccagcactcatgcactccagttcccacctcgttcactcacatgctcc

SEQ ID NO: 572 - DNA, 50 bp, Artificial Sequence
  Description of Artificial Sequence/note = Synthetic Construct
tcctggtcacctccccataactggccttccccacacccttctttctttgt

SEQ ID NO: 573 - DNA, 50 bp, Artificial Sequence
  Description of Artificial Sequence/note = Synthetic Construct
actccctaaacacactgcgcgtgctcaattcccaagggtaaggagggcac

SEQ ID NO: 574 - DNA, 50 bp, Artificial Sequence
  Description of Artificial Sequence/note = Synthetic Construct
aattgtggcaggagtcttaacagcagtgggatgttgtattatcccttgtg

SEQ ID NO: 575 - DNA, 50 bp, Artificial Sequence
  Description of Artificial Sequence/note = Synthetic Construct
tttgcccaccctttcccgattgattctttctgaataatgccttttaacca

SEQ ID NO: 576 - DNA, 50 bp, Artificial Sequence
  Description of Artificial Sequence/note = Synthetic Construct
caccagaaggaagaaactccgaacacatccgaacatcagaaggaacaaac

SEQ ID NO: 577 - DNA, 50 bp, Artificial Sequence
  Description of Artificial Sequence/note = Synthetic Construct
cagtcggtgctgtctcacyyttgagcagccnygctctgactcagctgtca

SEQ ID NO: 578 - DNA, 50 bp, Artificial Sequence
  Description of Artificial Sequence/note = Synthetic Construct
cccttgttaaatcctccttggttgtggtcattggactgtcacctgccaag

SEQ ID NO: 579 - DNA, 50 bp, Artificial Sequence
  Description of Artificial Sequence/note = Synthetic Construct
gggacaagaactcagaccttgctaaactaaggagtaagaagactgcaaca

SEQ ID NO: 580 - DNA, 50 bp, Artificial Sequence
  Description of Artificial Sequence/note = Synthetic Construct
gtcaaagtgcttcattaaatgggtcctgttccctgtgccacccaactggg

SEQ ID NO: 581 - DNA, 50 bp, Artificial Sequence
  Description of Artificial Sequence/note = Synthetic Construct
tcattcacgtggattcaatgtagtactyggtgtatggcaaattcaagttt

SEQ ID NO: 582 - DNA, 50 bp, Artificial Sequence
  Description of Artificial Sequence/note = Synthetic Construct
ctataaaagcctcccccttgcattccctcggtggagctcccgaaccactt

SEQ ID NO: 583 - DNA, 50 bp, Artificial Sequence
  Description of Artificial Sequence/note = Synthetic Construct
tgtacaccctgtgatattattcgtaatatcctagggggatgttactccta

SEQ ID NO: 584 - DNA, 50 bp, Artificial Sequence
  Description of Artificial Sequence/note = Synthetic Construct
gggnaaatgantgatattcagtaatggtgctgggacatttggttttccat

SEQ ID NO: 585 - DNA, 50 bp, Artificial Sequence
  Description of Artificial Sequence/note = Synthetic Construct
cccctctagaggatgcagcatwcaaggygccatcttggaagcagagasca

SEQ ID NO: 586 - DNA, 50 bp, Artificial Sequence
  Description of Artificial Sequence/note = Synthetic Construct
tggctgaacactcccagtaacagtggctctgcgtttctcggaggtggagc

SEQ ID NO: 587 - DNA, 50 bp, Artificial Sequence
  Description of Artificial Sequence/note = Synthetic Construct
catccaaacaagctgcgatattctacccaacgatatagaagctgtagttg

SEQ ID NO: 588 - DNA, 50 bp, Artificial Sequence
  Description of Artificial Sequence/note = Synthetic Construct
gcccacccaacccatcacagcttccagcaacaccaacatggactgcttgg

SEQ ID NO: 589 - DNA, 50 bp, Artificial Sequence
  Description of Artificial Sequence/note = Synthetic Construct
tggaagaggattctaagcctcagatgagaacacagccctagccaacacct

SEQ ID NO: 590 - DNA, 50 bp, Artificial Sequence
  Description of Artificial Sequence/note = Synthetic Construct
ttcttccagaccctcccaatcctaaagagattaactaagatctgaatagg

SEQ ID NO: 591 - DNA, 50 bp, Artificial Sequence
  Description of Artificial Sequence/note = Synthetic Construct
cgtgacctcctaggaatgagccttcctagtgatgtgggacctaaacttct

SEQ ID NO: 592 - DNA, 50 bp, Artificial Sequence
  Description of Artificial Sequence/note = Synthetic Construct
tttaaatttggagccctcaaaatcatcttcggagaaaggcatagacctgt

SEQ ID NO: 593 - DNA, 50 bp, Artificial Sequence
  Description of Artificial Sequence/note = Synthetic Construct
aaaacaancacnangagccgggggnggggaatcagtatccagagttgcta

SEQ ID NO: 594 - DNA, 50 bp, Artificial Sequence
  Description of Artificial Sequence/note = Synthetic Construct
cacacagacagcagattagggctaacctggcaaggatacagcttgtctgc

SEQ ID NO: 595 - DNA, 50 bp, Artificial Sequence
  Description of Artificial Sequence/note = Synthetic Construct
tctctttgtcttgtgtctttatttattacaatctctcgtctccgcacacg

SEQ ID NO: 596 - DNA, 50 bp, Artificial Sequence
  Description of Artificial Sequence/note = Synthetic Construct
aaccacaacatnagaggacccancactcctcctaccaccaaaacaaaacc

SEQ ID NO: 597 - DNA, 50 bp, Artificial Sequence
  Description of Artificial Sequence/note = Synthetic Construct
agaggctcatagaaatggcacttactaaaacctcccttaactatcctcca

SEQ ID NO: 598 - DNA, 50 bp, Artificial Sequence
  Description of Artificial Sequence/note = Synthetic Construct
cngatcctcccctcnagttgagccttgagatgagactgcagtcctggctg

SEQ ID NO: 599 - DNA, 50 bp, Artificial Sequence
  Description of Artificial Sequence/note = Synthetic Construct
tttggacccccaaaattctactggcaggaagcaggctgagaaaactactc

SEQ ID NO: 600 - DNA, 50 bp, Artificial Sequence
  Description of Artificial Sequence/note = Synthetic Construct
cagaggctcataaaaacggcacttactaaaacctcccttaactatcctcc

SEQ ID NO: 601 - DNA, 50 bp, Artificial Sequence
  Description of Artificial Sequence/note = Synthetic Construct
ttccctcccttgtccaggtgtgcgctcaccattgctccatctgtgagggt

SEQ ID NO: 602 - DNA, 50 bp, Artificial Sequence
  Description of Artificial Sequence/note = Synthetic Construct
ctaaagacactttgtgctcagacctagaaatcttctcaattggctgccat

SEQ ID NO: 603 - DNA, 50 bp, Artificial Sequence
  Description of Artificial Sequence/note = Synthetic Construct
ctggaaggcctatgcacctaataatagaacctcatgtatcttccgctact

SEQ ID NO: 604 - DNA, 50 bp, Artificial Sequence
  Description of Artificial Sequence/note = Synthetic Construct
aattaaccaaggcttttaaaattccttggccaaaagctcttccattggtt

SEQ ID NO: 605 - DNA, 50 bp, Artificial Sequence
  Description of Artificial Sequence/note = Synthetic Construct
catttcccgtttgccccaagaatactcttgtctctaatcctaatgtaaca

SEQ ID NO: 606 - DNA, 50 bp, Artificial Sequence
  Description of Artificial Sequence/note = Synthetic Construct
cccaggtggtttggcatttgattagaatgattgggctgccccaggtgtgt

SEQ ID NO: 607 - DNA, 50 bp, Artificial Sequence
  Description of Artificial Sequence/note = Synthetic Construct
aggatctggtccagacaggataaagtgaagaaacnrgcaggaaccagcag

SEQ ID NO: 608 - DNA, 50 bp, Artificial Sequence
  Description of Artificial Sequence/note = Synthetic Construct
cacngctccacacctgrcttnnccttggcaggnntggatcnaggnccttg

SEQ ID NO: 609 - DNA, 50 bp, Artificial Sequence
  Description of Artificial Sequence/note = Synthetic Construct
tgctttgcaataaaagcttcttgcctttcgcttcattctgactcatccct

SEQ ID NO: 610 - DNA, 50 bp, Artificial Sequence
  Description of Artificial Sequence/note = Synthetic Construct
aggagcatcttttgttctaatatttggtctttgaccctagttcctgacac

SEQ ID NO: 611 - DNA, 50 bp, Artificial Sequence
  Description of Artificial Sequence/note = Synthetic Construct
ccaacctcaccctttgtgtccatgctccttaattttcttggttgtgagac

SEQ ID NO: 612 - DNA, 50 bp, Artificial Sequence
  Description of Artificial Sequence/note = Synthetic Construct
tctgtttgcgggagaagtttctgactttacctggagctgagtcaakttag

SEQ ID NO: 613 - DNA, 50 bp, Artificial Sequence
  Description of Artificial Sequence/note = Synthetic Construct
aatctcatgtcaaaaaaacactagctgaacacaagctaaggaacagagac

SEQ ID NO: 614 - DNA, 50 bp, Artificial Sequence
  Description of Artificial Sequence/note = Synthetic Construct
ttgacactcactttcggttttgtgtattggcttcgtgacaccaaacaggg

SEQ ID NO: 615 - DNA, 50 bp, Artificial Sequence
  Description of Artificial Sequence/note = Synthetic Construct
gggaggagaccacccctcatattgtcttatgcccaatttctgcctccaaa

SEQ ID NO: 616 - DNA, 50 bp, Artificial Sequence
  Description of Artificial Sequence/note = Synthetic Construct
caccagaaggaagaaactccggacacatctgaacatctgaaggaacaaac

SEQ ID NO: 617 - DNA, 50 bp, Artificial Sequence
  Description of Artificial Sequence/note = Synthetic Construct
cactcctgaagtcagcgagaccacgaacccaccgggaggaacaaacaact

SEQ ID NO: 618 - DNA, 50 bp, Artificial Sequence
  Description of Artificial Sequence/note = Synthetic Construct
gtaagagagaattcctcctgcctgactgcctttgaactgggacatcggtc

SEQ ID NO: 619 - DNA, 50 bp, Artificial Sequence
  Description of Artificial Sequence/note = Synthetic Construct
taacaacatgtttttgctgcagataatcagccagagcctgtttctctrct

SEQ ID NO: 620 - DNA, 50 bp, Artificial Sequence
  Description of Artificial Sequence/note = Synthetic Construct
gaagtgacagccttgtgtgtgatctttctgccctccccaagtttgcattt

SEQ ID NO: 621 - DNA, 50 bp, Artificial Sequence
  Description of Artificial Sequence/note = Synthetic Construct
tcttgctgctaaaactgcatacaacagccacccagccaagaggaattaat

SEQ ID NO: 622 - DNA, 50 bp, Artificial Sequence
  Description of Artificial Sequence/note = Synthetic Construct
cccagctgccatgctaaaagaagctcaggctagactattggatgatgaga

SEQ ID NO: 623 - DNA, 50 bp, Artificial Sequence
  Description of Artificial Sequence/note = Synthetic Construct
gctgagaaaacttttgcctgagtgctggtttcactttgcggcaccaagca

SEQ ID NO: 624 - DNA, 50 bp, Artificial Sequence
  Description of Artificial Sequence/note = Synthetic Construct
cagaaactcaaaagaatgcaaccatttgtctctcacctacctgtgacctg

SEQ ID NO: 625 - DNA, 50 bp, Artificial Sequence
  Description of Artificial Sequence/note = Synthetic Construct
ctctagtatagcatcacatgacagatagcaggccctgaaagaaatcaaag

SEQ ID NO: 626 - DNA, 50 bp, Artificial Sequence
  Description of Artificial Sequence/note = Synthetic Construct
cntctctctcctgccgccttgtgaagaaggtgcttgcttcccctttgcct

SEQ ID NO: 627 - DNA, 50 bp, Artificial Sequence
  Description of Artificial Sequence/note = Synthetic Construct
cctccgtatgctgagcgccggtcccctgggcccactgttctttctctata

SEQ ID NO: 628 - DNA, 50 bp, Artificial Sequence
  Description of Artificial Sequence/note = Synthetic Construct
ttgagtatcccttatccaaaatgcttgggaccagaagtgtttcggatttc

SEQ ID NO: 629 - DNA, 50 bp, Artificial Sequence
  Description of Artificial Sequence/note = Synthetic Construct
gtgactccacatgttaatggtcttattcaagctaagcagcatctactatc

SEQ ID NO: 630 - DNA, 50 bp, Artificial Sequence
  Description of Artificial Sequence/note = Synthetic Construct
cgttgcaacgtgcacagttcatgctaaggatccgtgcgatgcactctgat

SEQ ID NO: 631 - DNA, 50 bp, Artificial Sequence
  Description of Artificial Sequence/note = Synthetic Construct
ngtcnattgtttgactttcacacattcgacttccatacacgttttcagga

SEQ ID NO: 632 - DNA, 50 bp, Artificial Sequence
  Description of Artificial Sequence/note = Synthetic Construct
tactgaatcagaatctgcgttttaacaagatccccaggtgattcatatgc

SEQ ID NO: 633 - DNA, 50 bp, Artificial Sequence
  Description of Artificial Sequence/note = Synthetic Construct
ttggccanaaaacttttnttgaatcttctcattgggaaaattgggagatc

SEQ ID NO: 634 - DNA, 50 bp, Artificial Sequence
  Description of Artificial Sequence/note = Synthetic Construct
ttcacgtgcactgattggacaataaacaaatacgtaagtacctcttctct

SEQ ID NO: 635 - DNA, 50 bp, Artificial Sequence
  Description of Artificial Sequence/note = Synthetic Construct
acttagaaaatttcgaggaaggcactccaaagcacggggtcccctgaggc

SEQ ID NO: 636 - DNA, 50 bp, Artificial Sequence
  Description of Artificial Sequence/note = Synthetic Construct
acgcatcaccttgcattgcttcccatccttccctgcctcacttccctttt

SEQ ID NO: 637 - DNA, 50 bp, Artificial Sequence
  Description of Artificial Sequence/note = Synthetic Construct
cgaagccaaacgatcatacacaacatacaccacagtcataccctcaaggg

SEQ ID NO: 638 - DNA, 50 bp, Artificial Sequence
  Description of Artificial Sequence/note = Synthetic Construct
agtagcgctgtcatcaatccaacctagattagataagttaacaagcaaga

SEQ ID NO: 639 - DNA, 50 bp, Artificial Sequence
  Description of Artificial Sequence/note = Synthetic Construct
cgccatgattgtgaggcctccccagccatgtggaactgtgagtccattaa

SEQ ID NO: 640 - DNA, 50 bp, Artificial Sequence
  Description of Artificial Sequence/note = Synthetic Construct
atgattgtaagtttcctgaggcctccccagaagccgagcagatgccagca

SEQ ID NO: 641 - DNA, 50 bp, Artificial Sequence
  Description of Artificial Sequence/note = Synthetic Construct
atgcggcccctcgaccttggacttcccagcctccagaactgtaagaaata

SEQ ID NO: 642 - DNA, 50 bp, Artificial Sequence
  Description of Artificial Sequence/note = Synthetic Construct
gccgtctacgaaccagggaatgagccctcaccagaaactgaatctgccgg

SEQ ID NO: 643 - DNA, 50 bp, Artificial Sequence
  Description of Artificial Sequence/note = Synthetic Construct
gccatctacaagccaaggagagaggcctcagaagaaaccaaccctgccga

SEQ ID NO: 644 - DNA, 50 bp, Artificial Sequence
  Description of Artificial Sequence/note = Synthetic Construct
catggaacagattctccctcacagccctcagaaggaaccaaccctgccga

SEQ ID NO: 645 - DNA, 50 bp, Artificial Sequence
  Description of Artificial Sequence/note = Synthetic Construct
tagcccagtgagacccatttcggacttctgacctccagaactgtaagata

SEQ ID NO: 646 - DNA, 50 bp, Artificial Sequence
  Description of Artificial Sequence/note = Synthetic Construct
ttgtgagaccctgaagcagaggacccagctaagctgtgcccggactcctg

SEQ ID NO: 647 - DNA, 50 bp, Artificial Sequence
  Description of Artificial Sequence/note = Synthetic Construct
catcttgactgcaacctcatgagagaccctgagccagaaccacccagcta

SEQ ID NO: 648 - DNA, 50 bp, Artificial Sequence
  Description of Artificial Sequence/note = Synthetic Construct
gttcttcagttttgggactcggactggctctccttgctcctcagcttgca

SEQ ID NO: 649 - DNA, 50 bp, Artificial Sequence
  Description of Artificial Sequence/note = Synthetic Construct
tcacgtgagccaattcccctaataaatcycytctatccatcctattggtt

SEQ ID NO: 650 - DNA, 50 bp, Artificial Sequence
  Description of Artificial Sequence/note = Synthetic Construct
ccacaatcgcgtgagccaattccttaaaataaatctctctctacacacac

SEQ ID NO: 651 - DNA, 50 bp, Artificial Sequence
  Description of Artificial Sequence/note = Synthetic Construct
tctgcctgcctgatngtcttcgaactggaatatcagctctgcggattttg

SEQ ID NO: 652 - DNA, 50 bp, Artificial Sequence
  Description of Artificial Sequence/note = Synthetic Construct
taaaascaagctgtrccccgaccaccttgggcacatgtcgtcaggacctc

SEQ ID NO: 653 - DNA, 50 bp, Artificial Sequence
  Description of Artificial Sequence/note = Synthetic Construct
ctaaaatgtataaaascaagctgtrccccgaccaccttgggcacatgtkg

SEQ ID NO: 654 - DNA, 50 bp, Artificial Sequence
  Description of Artificial Sequence/note = Synthetic Construct
attgaagccctcaaaatcatctttggagaaaggcacagaccacagatgtt

SEQ ID NO: 655 - DNA, 50 bp, Artificial Sequence; identical to SEQ ID NO: 81
  Description of Artificial Sequence/note = Synthetic Construct

SEQ ID NO: 656 - DNA, 50 bp, Artificial Sequence
  Description of Artificial Sequence/note = Synthetic Construct
cacggtcctaccgatatgtgatgtcaccccyggaggcccagctgtaaaat

SEQ ID NO: 657 - DNA, 50 bp, Artificial Sequence
  Description of Artificial Sequence/note = Synthetic Construct
ccggatrcccagctttaaaatttctctcttttgtactctgtccctttatt

SEQ ID NO: 658 - DNA, 50 bp, Artificial Sequence
  Description of Artificial Sequence/note = Synthetic Construct
ggtctttgggtcttcatttctgaaggctcccatgtcacgtaaaactttga

SEQ ID NO: 659 - DNA, 50 bp, Artificial Sequence; identical to SEQ ID NO: 95
  Description of Artificial Sequence/note = Synthetic Construct

SEQ ID NO: 660 - DNA, 50 bp, Artificial Sequence; identical to SEQ ID NO: 96
  Description of Artificial Sequence/note = Synthetic Construct

SEQ ID NO: 661 - DNA, 50 bp, Artificial Sequence
  Description of Artificial Sequence/note = Synthetic Construct
tgcgggatggccaccttgcaggctgtaaccctttataagaaataaagtct

SEQ ID NO: 662 - DNA, 50 bp, Artificial Sequence
  Description of Artificial Sequence/note = Synthetic Construct
tgccttttctccwattaatctgccttttgtsagttgatttttcagtgaam

SEQ ID NO: 663 - DNA, 50 bp, Artificial Sequence; identical to SEQ ID NO: 128
  Description of Artificial Sequence/note = Synthetic Construct

SEQ ID NO: 664 - DNA, 50 bp, Artificial Sequence; identical to SEQ ID NO: 150
  Description of Artificial Sequence/note = Synthetic Construct

SEQ ID NO: 665 - DNA, 50 bp, Artificial Sequence
  Description of Artificial Sequence/note = Synthetic Construct
gccggaaactctaagagggtagaggwaaaatttttccttcyctnccatgg

SEQ ID NO: 666 - DNA, 50 bp, Artificial Sequence
  Description of Artificial Sequence/note = Synthetic Construct
tttacactgtggaatcaccctgaattctttcttgcatgagatccaagaac

SEQ ID NO: 667 - DNA, 50 bp, Artificial Sequence
  Description of Artificial Sequence/note = Synthetic Construct
tgctctaaaacttgcctcggtctctttttctgccttatgcccctcagtcg

SEQ ID NO: 668 - DNA, 50 bp, Artificial Sequence; identical to SEQ ID NO: 159
  Description of Artificial Sequence/note = Synthetic Construct

SEQ ID NO: 669 - DNA, 50 bp, Artificial Sequence
  Description of Artificial Sequence/note = Synthetic Construct
gtgtatgccccaaattgcaattctgttcttcacatgttattcccaaataa

SEQ ID NO: 670 - DNA, 50 bp, Artificial Sequence
  Description of Artificial Sequence/note = Synthetic Construct
agccgcttcaataaaagttgctgtctaataccaccarctcgcccttgaat

SEQ ID NO: 671 - DNA, 50 bp, Artificial Sequence; identical to SEQ ID NO: 670
  Description of Artificial Sequence/note = Synthetic Construct

SEQ ID NO: 672 - DNA, 50 bp, Artificial Sequence
  Description of Artificial Sequence/note = Synthetic Construct
attctccctttaaaacgcccagtcacctctgcacaaatcgaagctgagct

SEQ ID NO: 673 - DNA, 50 bp, Artificial Sequence
  Description of Artificial Sequence/note = Synthetic Construct
cctcattctccctttaaaacgcccagtcacctctgcacaaattggaatgg

SEQ ID NO: 674 - DNA, 50 bp, Artificial Sequence
  Description of Artificial Sequence/note = Synthetic Construct
tagcagattgcctgtgatgcgcatcacattctggtttaatgcttattcaa

SEQ ID NO: 675 - DNA, 50 bp, Artificial Sequence
  Description of Artificial Sequence/note = Synthetic Construct
cctgtgagtcctcctagcgaatcaccgaacctgggggtggtcttgggaac

SEQ ID NO: 676 - DNA, 50 bp, Artificial Sequence; identical to SEQ ID NO: 167
  Description of Artificial Sequence/note = Synthetic Construct

SEQ ID NO: 677 - DNA, 50 bp, Artificial Sequence; identical to SEQ ID NO: 170
  Description of Artificial Sequence/note = Synthetic Construct

SEQ ID NO: 678 - DNA, 50 bp, Artificial Sequence; identical to SEQ ID NO: 171
  Description of Artificial Sequence/note = Synthetic Construct

SEQ ID NO: 679 - DNA, 50 bp, Artificial Sequence
  Description of Artificial Sequence/note = Synthetic Construct
gctgcaaccctttatgagaaataaagctctcctttccaaatttatgaacc

SEQ ID NO: 680 - DNA, 50 bp, Artificial Sequence; identical to SEQ ID NO: 173
  Description of Artificial Sequence/note = Synthetic Construct

SEQ ID NO: 681 - DNA, 50 bp, Artificial Sequence
  Description of Artificial Sequence/note = Synthetic Construct
aagcatgattaatacaakytgctctgtgatgaacggatgccaaatagwcg

SEQ ID NO: 682 - DNA, 50 bp, Artificial Sequence; identical to SEQ ID NO: 176
  Description of Artificial Sequence/note = Synthetic Construct

SEQ ID NO: 683 - DNA, 50 bp, Artificial Sequence
  Description of Artificial Sequence/note = Synthetic Construct
cttctagcgaatcactgaacctgagggtggtcttggggacccccgacaca

SEQ ID NO: 684 - DNA, 50 bp, Artificial Sequence
  Description of Artificial Sequence/note = Synthetic Construct
gcgtcttgactgcgccgataccacgtgggacagagawgaactrcccagct

SEQ ID NO: 685 - DNA, 50 bp, Artificial Sequence; identical to SEQ ID NO: 181
  Description of Artificial Sequence/note = Synthetic Construct

SEQ ID NO: 686 - DNA, 50 bp, Artificial Sequence; identical to SEQ ID NO: 182
  Description of Artificial Sequence/note = Synthetic Construct

SEQ ID NO: 687 - DNA, 50 bp, Artificial Sequence
  Description of Artificial Sequence/note = Synthetic Construct
ataaacttgctcttctcactgtactccgcaactcgccttgaattccttcc

SEQ ID NO: 688 - DNA, 50 bp, Artificial Sequence
  Description of Artificial Sequence/note = Synthetic Construct
ctctgcttttgttgcttcattctttccttgctttgtttgtgcgttttgtc

SEQ ID NO: 689 - DNA, 50 bp, Artificial Sequence; identical to SEQ ID NO: 688
  Description of Artificial Sequence/note = Synthetic Construct

SEQ ID NO: 690 - DNA, 50 bp, Artificial Sequence; identical to SEQ ID NO: 196
  Description of Artificial Sequence/note = Synthetic Construct

SEQ ID NO: 691 - DNA, 50 bp, Artificial Sequence; identical to SEQ ID NO: 197
  Description of Artificial Sequence/note = Synthetic Construct

SEQ ID NO: 692 - DNA, 50 bp, Artificial Sequence
  Description of Artificial Sequence/note = Synthetic Construct
acctccaaccttctctttgttctttggacataccgaagaccacctggtct

SEQ ID NO: 693 - DNA, 50 bp, Artificial Sequence
  Description of Artificial Sequence/note = Synthetic Construct
acaactgtcttggtaaattatttttacctcccgcgccaccggccccagat

SEQ ID NO: 694 - DNA, 50 bp, Artificial Sequence; identical to SEQ ID NO: 204
  Description of Artificial Sequence/note = Synthetic Construct

SEQ ID NO: 695 - DNA, 50 bp, Artificial Sequence; identical to SEQ ID NO: 219
  Description of Artificial Sequence/note = Synthetic Construct

SEQ ID NO: 696 - DNA, 50 bp, Artificial Sequence; identical to SEQ ID NO: 220
  Description of Artificial Sequence/note = Synthetic Construct

SEQ ID NO: 697 - DNA, 50 bp, Artificial Sequence; identical to SEQ ID NO: 221
  Description of Artificial Sequence/note = Synthetic Construct

SEQ ID NO: 698 - DNA, 50 bp, Artificial Sequence; identical to SEQ ID NO: 222
  Description of Artificial Sequence/note = Synthetic Construct

SEQ ID NO: 699 - DNA, 50 bp, Artificial Sequence
  Description of Artificial Sequence/note = Synthetic Construct
agccatttgggtgtggtgtagaactggaaactgtgtcaagggtgactgag

SEQ ID NO: 700 - DNA, 50 bp, Artificial Sequence
  Description of Artificial Sequence/note = Synthetic Construct
aaattcccacttgcccatgctgtattcggagttgagcccaatctctctcc

SEQ ID NO: 701 - DNA, 50 bp, Artificial Sequence
  Description of Artificial Sequence/note = Synthetic Construct
tccccacttgtccttgctgtattcggagttgagcccaatctctctcccct

SEQ ID NO: 702 - DNA, 50 bp, Artificial Sequence; identical to SEQ ID NO: 226
  Description of Artificial Sequence/note = Synthetic Construct

SEQ ID NO: 703 - DNA, 50 bp, Artificial Sequence
  Description of Artificial Sequence/note = Synthetic Construct
ttgtactctgtccctttatttctcaagccagccgacgcttagggaaaata

SEQ ID NO: 704 - DNA, 50 bp, Artificial Sequence
  Description of Artificial Sequence/note = Synthetic Construct
actatcttgtgtgtgtctattatttctcaacctgccgatccgcctaggag

SEQ ID NO: 705 - DNA, 50 bp, Artificial Sequence
  Description of Artificial Sequence/note = Synthetic Construct
cgcccaataaattctgctcctcacccttcaatgtgtccgcgwgcctaatc

SEQ ID NO: 706 - DNA, 50 bp, Artificial Sequence
  Description of Artificial Sequence/note = Synthetic Construct
gkgacaagaacccgggttttagctgaactaaggagcaaaatyctgcawca

SEQ ID NO: 707 - DNA, 50 bp, Artificial Sequence
  Description of Artificial Sequence/note = Synthetic Construct
gttcctgaggtcggagcgttctccctattgcaatagtctttttgaataaa

SEQ ID NO: 708 - DNA, 50 bp, Artificial Sequence; identical to SEQ ID NO: 252
  Description of Artificial Sequence/note = Synthetic Construct

SEQ ID NO: 709 - DNA, 50 bp, Artificial Sequence; identical to SEQ ID NO: 253
  Description of Artificial Sequence/note = Synthetic Construct

SEQ ID NO: 710 - DNA, 50 bp, Artificial Sequence
  Description of Artificial Sequence/note = Synthetic Construct
cttcctcatncaccytataaaagcctttccttcaagcccctccggcggag

SEQ ID NO: 711 - DNA, 50 bp, Artificial Sequence; identical to SEQ ID NO: 255
  Description of Artificial Sequence/note = Synthetic Construct

SEQ ID NO: 712 - DNA, 50 bp, Artificial Sequence; identical to SEQ ID NO: 277
  Description of Artificial Sequence/note = Synthetic Construct

SEQ ID NO: 713 - DNA, 50 bp, Artificial Sequence; identical to SEQ ID NO: 278
  Description of Artificial Sequence/note = Synthetic Construct

SEQ ID NO: 714 - DNA, 50 bp, Artificial Sequence
  Description of Artificial Sequence/note = Synthetic Construct
tgtgggacttcaccttgtgatcgtgtgagtcaatactccttaataaactc

SEQ ID NO: 715 - DNA, 50 bp, Artificial Sequence; identical to SEQ ID NO: 301
  Description of Artificial Sequence/note = Synthetic Construct

SEQ ID NO: 716 - DNA, 50 bp, Artificial Sequence
  Description of Artificial Sequence/note = Synthetic Construct
gccacagaggtttccggccagaaaagcgacaccccaaggatcccatgaca

SEQ ID NO: 717 - DNA, 50 bp, Artificial Sequence
  Description of Artificial Sequence/note = Synthetic Construct
acactaaataaagctcttcttcgtcttcttcacccttcacttgtctgcgt

SEQ ID NO: 718 - DNA, 50 bp, Artificial Sequence
  Description of Artificial Sequence/note = Synthetic Construct
ttgargtctcccggttcgcgarctgtwctttctctyattgtatgcacaat

SEQ ID NO: 719 - DNA, 50 bp, Artificial Sequence; identical to SEQ ID NO: 323
  Description of Artificial Sequence/note = Synthetic Construct

SEQ ID NO: 720 - DNA, 50 bp, Artificial Sequence
  Description of Artificial Sequence/note = Synthetic Construct
agctacccctggacttttcagttacgtgaaccaataaattcccttttttg

SEQ ID NO: 721 - DNA, 50 bp, Artificial Sequence; identical to SEQ ID NO: 337
  Description of Artificial Sequence/note = Synthetic Construct

SEQ ID NO: 722 - DNA, 50 bp, Artificial Sequence
  Description of Artificial Sequence/note = Synthetic Construct
tttctgactcatccttgaattccttctcgcgatggtgtcaagagcctgga

SEQ ID NO: 723 - DNA, 50 bp, Artificial Sequence; identical to SEQ ID NO: 343
  Description of Artificial Sequence/note = Synthetic Construct

SEQ ID NO: 724 - DNA, 50 bp, Artificial Sequence
  Description of Artificial Sequence/note = Synthetic Construct
tgatttcagccttgtgagaccctgagcagaggacccagctaagccgtgcc

SEQ ID NO: 725 - DNA, 50 bp, Artificial Sequence
  Description of Artificial Sequence/note = Synthetic Construct
agccactgtacattttggggtttatttgttacagcagctagcgttacctt

SEQ ID NO: 726 - DNA, 50 bp, Artificial Sequence; identical to SEQ ID NO: 380
  Description of Artificial Sequence/note = Synthetic Construct

SEQ ID NO: 727 - DNA, 50 bp, Artificial Sequence
  Description of Artificial Sequence/note = Synthetic Construct
ttgatttcggccttgtgagaccctgagcagagaacccagccgagcccacc

SEQ ID NO: 728 - DNA, 50 bp, Artificial Sequence
  Description of Artificial Sequence/note = Synthetic Construct
tgcccaaattgcagattcgtgagcaaaataaatgattgttgttgttttaa

SEQ ID NO: 729 - DNA, 50 bp, Artificial Sequence
  Description of Artificial Sequence/note = Synthetic Construct
ctcagctttgcttgatcaacaggttttnttttctggtggtctttttgggg

SEQ ID NO: 730 - DNA, 50 bp, Artificial Sequence
  Description of Artificial Sequence/note = Synthetic Construct
tggtgctcycccttaccacagtaagcaataaactcagctttgtcttatca

SEQ ID NO: 731 - DNA, 50 bp, Artificial Sequence
  Description of Artificial Sequence/note = Synthetic Construct
gagagaccctgagccagaaccacccagctaagctgctcccgaattcctga

SEQ ID NO: 732 - DNA, 50 bp, Artificial Sequence
  Description of Artificial Sequence/note = Synthetic Construct
ggctgtgtctccctggtttgcaaactgttcactggaataaactctcctcc

SEQ ID NO: 733 - DNA, 50 bp, Artificial Sequence
  Description of Artificial Sequence/note = Synthetic Construct
ccctgctgtgccctgtccgaattcctgacccacagaatccgtgagcataa

SEQ ID NO: 734 - DNA, 50 bp, Artificial Sequence
  Description of Artificial Sequence/note = Synthetic Construct
agatgctcgcaccatgctttttgtccagccagcagaaytatgagccaaat

SEQ ID NO: 735 - DNA, 50 bp, Artificial Sequence
  Description of Artificial Sequence/note = Synthetic Construct
agccttcaagtcttcccagctgaggccccagacatcatggagcagagaca

SEQ ID NO: 736 - DNA, 50 bp, Artificial Sequence
  Description of Artificial Sequence/note = Synthetic Construct
tgcccttgaacttcccagcctgcagaaccatgagctaaataaacctcttt

SEQ ID NO: 737 - DNA, 50 bp, Artificial Sequence
  Description of Artificial Sequence/note = Synthetic Construct
gcctccagagggagcatggccctgctgacaccttkgatttcagcccagtg

SEQ ID NO: 738 - DNA, 50 bp, Artificial Sequence
  Description of Artificial Sequence/note = Synthetic Construct
gatgacgcagcaagaaggccctcaccagatgccggcnccwtgatcttgga

SEQ ID NO: 739 - DNA, 50 bp, Artificial Sequence
  Description of Artificial Sequence/note = Synthetic Construct
tctcgctttaataaattcctgctttcgctgcttcgttcctgtgtttcatt

SEQ ID NO: 740 - DNA, 50 bp, Artificial Sequence
  Description of Artificial Sequence/note = Synthetic Construct
tggtgtgagagcagaggaaaaacacggtttgagagagttttcccgaaaca

SEQ ID NO: 741 - DNA, 50 bp, Artificial Sequence
  Description of Artificial Sequence/note = Synthetic Construct
tctgtcttttgttacaggggtctattccaactaagaacttatgagggttg

SEQ ID NO: 742 - DNA, 50 bp, Artificial Sequence
  Description of Artificial Sequence/note = Synthetic Construct
tatctggatcgaccacattgaggaactgggaggaggcggagaactggaaa

SEQ ID NO: 743 - DNA, 50 bp, Artificial Sequence; identical to SEQ ID NO: 448
  Description of Artificial Sequence/note = Synthetic Construct

SEQ ID NO: 744 - DNA, 50 bp, Artificial Sequence
  Description of Artificial Sequence/note = Synthetic Construct
ctcattttcctcttgccgccgccatgtaagaagtgcctttcgcctcccgc

SEQ ID NO: 745 - DNA, 50 bp, Artificial Sequence
  Description of Artificial Sequence/note = Synthetic Construct
atgtgaagaaggacgtgtttgcttccccttccgccatgattgtaagtttc

SEQ ID NO: 746 - DNA, 50 bp, Artificial Sequence
  Description of Artificial Sequence/note = Synthetic Construct
atgattgnaagcttcctgaggcctcaccagaagccgagcagatgccggcg

SEQ ID NO: 747 - DNA, 50 bp, Artificial Sequence
  Description of Artificial Sequence/note = Synthetic Construct
gccatgcttcttgtacagcctgcagaaccgtgagccaaataaacctcttt

SEQ ID NO: 748 - DNA, 50 bp, Artificial Sequence
  Description of Artificial Sequence/note = Synthetic Construct
ctgtggagtgtactttcgcttcaataaatctgtgctttcgttactncgtt

SEQ ID NO: 749 - DNA, 50 bp, Artificial Sequence
  Description of Artificial Sequence/note = Synthetic Construct
tgggtggcaccacagttccgagaaatcttcacctttttccaggaatcttc

SEQ ID NO: 750 - DNA, 50 bp, Artificial Sequence; identical to SEQ ID NO: 564
  Description of Artificial Sequence/note = Synthetic Construct

SEQ ID NO: 751 - DNA, 50 bp, Artificial Sequence
  Description of Artificial Sequence/note = Synthetic Construct
tccttttacccctccctcaaagtgctttgctctcagcttctgccagaggc

SEQ ID NO: 752 - DNA, 50 bp, Artificial Sequence
  Description of Artificial Sequence/note = Synthetic Construct
ttgttacaggggtctgtcccagctaagaactatgaagggtagagagaaaa

SEQ ID NO: 753 - DNA, 50 bp, Artificial Sequence
  Description of Artificial Sequence/note = Synthetic Construct
gatatgccgcyggtaactcagggtaactcggatctcttccaccggtaaca

SEQ ID NO: 754 - DNA, 50 bp, Artificial Sequence; identical to SEQ ID NO: 582
  Description of Artificial Sequence/note = Synthetic Construct

SEQ ID NO: 755 - DNA, 50 bp, Artificial Sequence
  Description of Artificial Sequence/note = Synthetic Construct
catcttggaagcagagascaggccctcaccagacaccaaacctgctggna

SEQ ID NO: 756 - DNA, 50 bp, Artificial Sequence
  Description of Artificial Sequence/note = Synthetic Construct
cttgtgagaccctgagcagaggacccagctaagctgtgcccagactcctg

SEQ ID NO: 757 - DNA, 50 bp, Artificial Sequence
  Description of Artificial Sequence/note = Synthetic Construct
tcacgggccatggtcactcatatttggctcagaataaatctcttcaaata

SEQ ID NO: 758 - DNA, 50 bp, Artificial Sequence; identical to SEQ ID NO: 592
  Description of Artificial Sequence/note = Synthetic Construct

SEQ ID NO: 759 - DNA, 50 bp, Artificial Sequence
  Description of Artificial Sequence/note = Synthetic Construct
acaccttgattgcagccttgtgagagaccctgagccagaagacccaacta

SEQ ID NO: 760 - DNA, 50 bp, Artificial Sequence
  Description of Artificial Sequence/note = Synthetic Construct
cttctcagcctccataatcaagtgagccaattcccctaataaatcccttc

SEQ ID NO: 761 - DNA, 50 bp, Artificial Sequence
  Description of Artificial Sequence/note = Synthetic Construct
gagcagtaccgttcaataaaagattgctgtctaacaccactggctcaccc

SEQ ID NO: 762 - DNA, 50 bp, Artificial Sequence
  Description of Artificial Sequence/note = Synthetic Construct
ctcaggcaaagghaccachgghcacagaggtttctggccagaaaagbgac

SEQ ID NO: 763 - DNA, 50 bp, Artificial Sequence; identical to SEQ ID NO: 609
  Description of Artificial Sequence/note = Synthetic Construct

SEQ ID NO: 764 - DNA, 50 bp, Artificial Sequence
  Description of Artificial Sequence/note = Synthetic Construct
tgtgggatctgatgctaactccagggtagatagtgtcagaattgaattaa

SEQ ID NO: 765 - DNA, 50 bp, Artificial Sequence
  Description of Artificial Sequence/note = Synthetic Construct
cctgggtctccagcttgccaactcaccctgcagatcttgggacttctcag

SEQ ID NO: 766 - DNA, 50 bp, Artificial Sequence
  Description of Artificial Sequence/note = Synthetic Construct
taaatatgtgggtcaaactctgtttgtggctctcagctctgaaggctgtt

SEQ ID NO: 767 - DNA, 50 bp, Artificial Sequence
  Description of Artificial Sequence/note = Synthetic Construct
tacaccatgtggagcagaagaaccacccagctgagcccagccaacacaga

SEQ ID NO: 768 - DNA, 50 bp, Artificial Sequence
  Description of Artificial Sequence/note = Synthetic Construct
aaaaccaagctgtgctctgaccaccttgggcacatgtcgtcaggacctcc

SEQ ID NO: 769 - DNA, 50 bp, Artificial Sequence
  Description of Artificial Sequence/note = Synthetic Construct
tcanaggccatggtcactcatatttggctcagaataaatctcttcaaata

SEQ ID NO: 770 - DNA, 50 bp, Artificial Sequence
  Description of Artificial Sequence/note = Synthetic Construct
tgcttgcttcccctttgccttctgccatgattgtaagtttcctgaggcct

SEQ ID NO: 771 - DNA, 20 bp, Artificial Sequence
  Description of Artificial Sequence/note = Synthetic Construct
ccaccactgctgtttgccac

SEQ ID NO: 772 - DNA, 23 bp, Artificial Sequence
  Description of Artificial Sequence/note = Synthetic Construct
gcctcgtgttctctgacctgggg

SEQ ID NO: 773 - DNA, 20 bp, Artificial Sequence
  Description of Artificial Sequence/note = Synthetic Construct
cgggtgatttctgcatttcc

SEQ ID NO: 774 - DNA, 20 bp, Artificial Sequence
  Description of Artificial Sequence/note = Synthetic Construct
gacatttaagtctgcagagg

SEQ ID NO: 775 - DNA, 20 bp, Artificial Sequence
  Description of Artificial Sequence/note = Synthetic Construct
ttggcggtatcacaacctct

SEQ ID NO: 776 - DNA, 24 bp, Artificial Sequence
  Description of Artificial Sequence/note = Synthetic Construct
tcataaagcaagtcctcagtgacc

SEQ ID NO: 777 - DNA, 18 bp, Artificial Sequence
  Description of Artificial Sequence/note = Synthetic Construct
gtgacgattccggattga

SEQ ID NO: 778 - DNA, 24 bp, Artificial Sequence
  Description of Artificial Sequence/note = Synthetic Construct
ggggtggagagttctgtagatgtc

(19) United States
(12) Patent Application Publication
(10) Pub. No.: US 2006/0115806 A1
(43) Pub. Date: Jun. 1, 2006

(54) Global analysis of transposable elements as molecular markers of cancer

(75) Inventor: John F McDonald, Arnoldsville, GA (US)

Correspondence Address: NEEDLE & ROSENBERG, P.C., SUITE 1000, 999 PEACHTREE STREET, ATLANTA, GA, 30309-3915, US

(73) Assignee: UNIVERSITY OF GEORGIA RESEARCH FOUNDATION, INC., Athens, GA (US)

(21) Appl. No.: 10/554,711

(22) PCT Filed: Apr. 29, 2004
(86) PCT No.: PCT/US04/13522

§ 371 Date: Jan. 19, 2006

Related U.S. Application Data
(60) Provisional application No. 60/466,798, filed on Apr. 29, 2003.

(51) Int. Cl. C12Q 1/70; C12Q 1/68; C12N 15/86

(52) U.S. Cl. 435/5; 435/6; 435/456

(57) ABSTRACT

The present invention provides methods of determining expression patterns, methylation patterns and chromatin status patterns for transposable element gene sequences. These methods can be utilized to diagnose, stage and treat cancer.

This application claims priority to U.S. provisional application Ser. No. 60/466,798, filed Apr. 29, 2003, which is herein incorporated by this reference in its entirety.

### FIELD OF THE INVENTION

This invention relates to the determination of expression patterns, DNA methylation patterns and chromatin properties of families of transposable elements in order to detect, classify, characterize and treat cancer.

### BACKGROUND

The human genome comprises numerous families of transposable elements, such as DNA elements, i.e. Charlie- and Tigger groups (see Smit (1999) Interspersed repeats and other mementos of transposable elements in mammalian genomes. Current Opinion in Genetics & Development, 9: 657-663) and retroelements, i.e., LINEs (long interspersed nuclear elements), SINES (short interspersed nuclear elements) and HERVs (human endogenous retroviruses). To date, over 50 families of retroviral elements have been identified and the members of these families make up greater than 43% of the genome (See Li et al. (2001) Evolutionary analysis of the human genome. Nature, 409 (6822): 847-9). Some families can include hundreds to thousands of retroelements and the expression of retroelements genes is normally suppressed. However, under certain conditions, such as cancer, retroelements may no longer be suppressed and expression of retroelement genes is activated, concomitant with changes in DNA methylation patterns and/or chromatin states.

The present invention provides methods of determining patterns of transposable element expression, transposable element methylation and chromatin status of transposable elements within the genome such that these patterns can be used to diagnose cancer, identify a type of cancer, classify a cancer at a particular stage and measure progression of cancer. All of the methods of the present invention can be utilized to analyze full-length transposable element sequences or fragments thereof. These transposable elements include retroelements and fragments thereof as well as DNA elements and fragments thereof from mammalian species. Thus, the present invention provides methods of determining patterns of retroelement expression, retroelement methylation and chromatin status of retroelements within the genome such that these patterns can be used to diagnose cancer, identify a type of cancer, classify a cancer at a particular stage and measure progression of cancer. Also provided are methods of determining DNA element expression, DNA element methylation and chromatin state of DNA elements within the genome such that these patterns can be used to diagnose cancer, identify a type of cancer, classify a cancer at a particular stage and measure progression of cancer.

### SUMMARY OF THE INVENTION

The present invention provides a method of determining an expression pattern of one or more families of transposable elements in a sample comprising determining expression of one or more families of transposable elements.

Also provided by the present invention is a method of assigning an expression pattern of transposable elements to a type of cancerous cell in a sample, comprising: a) determining expression of one or more families of transposable elements; and b) assigning the expression pattern obtained from step a) to the type of cancerous cell in the sample.

Further provided by the present invention is a method of diagnosing cancer comprising: a) determining expression of one or more families of transposable elements in a sample to obtain an expression pattern; b) matching the expression pattern of step a) with a known expression pattern for a type of cancer; and c) diagnosing the type of cancer based on matching of the expression pattern of a) with a known expression pattern for a type of cancer.

The present invention also provides a method of determining the effectiveness of an anti-cancer therapeutic in a subject comprising: a) determining expression of one or more families of transposable elements, in a sample obtained from the subject, to obtain a first expression pattern; b) administering an anti-cancer therapeutic to the subject; c) determining expression of one or more families of transposable elements in a sample obtained from the subject after administration of an anti-cancer therapeutic to obtain a second expression pattern; and d) comparing the second expression pattern with the first expression pattern such that if transposable elements are differentially expressed in the second expression pattern as compared to the first expression pattern, the anti-cancer therapeutic is an effective anti-cancer therapeutic.

Also provided by the present invention is a method of determining a methylation pattern of one or more families of transposable elements in a sample comprising determining methylation of one or more families of transposable elements.

The present invention also provides a method of assigning a methylation pattern of transposable elements to a type of cancerous cell in a sample, comprising: a) determining methylation of one or more families of transposable elements; and b) assigning the methylation pattern obtained from step a) to the type of cancerous cell in the sample.

Also provided by the present invention is a method of diagnosing cancer comprising: a) determining methylation of one or more families of transposable elements in a sample to obtain a methylation pattern; b) comparing the methylation pattern of step a) with a known methylation pattern for a type of cancer; and c) diagnosing the type of cancer based on matching of the methylation pattern of a) with a known methylation pattern for a type of cancer.

The present invention also provides a method of determining the effectiveness of an anti-cancer therapeutic in a subject comprising: a) determining methylation of one or more families of transposable elements, in a sample obtained from the subject, to obtain a first methylation pattern; b) administering an anti-cancer therapeutic to the subject; c) determining methylation of one or more families of transposable elements in a sample obtained from the subject after administration of an anti-cancer therapeutic to obtain a second methylation pattern; and d) comparing the second methylation pattern with the first methylation pattern such that if there is a change in the second methylation pattern as compared to the first methylation pattern, the anti-cancer therapeutic is an effective anti-cancer therapeutic.

### BRIEF DESCRIPTION OF THE FIGURES

FIG. 1 shows RT-PCR from normal and tumor ovarian samples comparing expression levels of HERV-K and HERV-W. (−) indicates a control without reverse transcriptase documenting absence of relevant DNA contamination. No Herv K or Herv W expression was detectable in this normal sample, HervW expression and even higher HervK expression was detected in this ovarian carcinoma sample.

FIG. 2 is a southern blot analysis of genomic DNA after digest with MspI (N) or its methylation-sensitive isoschizomer HpaII (H), resp., hybridized with a HERV-W probe spanning the putative promoter region of the element. Equal amounts of DNA were loaded per sample, i.e. MspI/HpaII pair. Fragment sizes range from >0.1 kb to >3.0 kb. Samples represent ovarian carcinoma (T—malignant), ovarian adenoma (B—benign), borderline ovarian tumor (LMP) and non-tumor ovarian tissue (N). Fragments between 0.3 kb and 1 kb appear in most of the malignant samples in the HpaII digests, but not in adenoma, borderline or non-tumor samples, indicating extensive cytosine methylation of this particular HervW region in non-carcinoma ovarian tissue and loss of HervW methylation in ovarian carcinoma. See region defined by arrows.

FIG. 3 is a southern blot analysis of genomic DNA after digest with MspI (M) or its methylation-sensitive isoschizomer HpaII (H), resp., hybridized with a LINE1 probe spanning the putative promoter region of the element. Equal amounts of DNA were loaded per sample, i.e. per MspI/HpaII pair. Fragment sizes range from 0.1 kb to >3.0 kb. Samples represent ovarian carcinoma (T—malignant), borderline ovarian tumor (B) and non-tumor ovarian tissue (N).

FIG. 4 shows hypomethylation and expression of L1 and HERV-W elements in ovarian cancer. Genomic DNA was digested either with MspI (left) or HpaII (right), and hybridized with probes specific for the promoter regions of L1 (A) or HERV-W (B) elements. The restriction enzymes MspI and HpaII recognize the sequence CCGG but HpaII only cuts when the recognition sequence is unmethylated at the inner cytosine (i.e., CCGG) while MspI is indifferent to the methylation status of the inner cytosine. Brackets indicate bands from restriction cut sites internal to the elements (B=benign cystic mass; LMP=low-malignancy potential or borderline tumor; N=normal ovary. (C) Real time RT-PCR was performed to determine expression levels of LINE-1 and HERV-W elements in representative malignant and non-malignant samples. Normalized values (retroelement expression value divided by expression value of the RPS27A control gene. Shown is the average of 3 replicate assays per sample USE. Ribosomal protein S27A (RPS27A) expression has been previously determined to be unchanged between the malignant and non-malignant samples examined in this study.

FIG. 5 is an example of an array that was utilized to assess retroelements patterns in cancer cells. Each dot represents a hybridization of the labeled RNA pool (from either a cancer or control sample in this case a cancer sample) to the “spots” representing retroelement sequences. A bright color indicates that the element was expressed in this sample. The intensity of the dot is correlated with the level of expression. In this array, 3 replicate copies of the elements (spots) are aligned vertically. Different elements families are arranged side by side.

### DETAILED DESCRIPTION OF THE INVENTION

The present invention may be understood more readily by reference to the following detailed description of the preferred embodiments of the invention and the Examples included therein.

Before methods are disclosed and described, it is to be understood that this invention is not limited to specific methods, as such may, of course, vary. It is also to be understood that the terminology used herein is for the purpose of describing particular embodiments only and is not intended to be limiting.

It must be noted that, as used in the specification and the appended claims, the singular forms “a,” “an,” and “the” include plural referents unless the context clearly dictates otherwise. Thus, for example, reference to “a nucleic acid” includes multiple copies of the nucleic acid and can also include more than one particular species of nucleic acid molecule. Similarly, reference to “a cell” includes one or more cells, including populations of cells.

Analysis of Expression Patterns

The present invention provides a method of determining an expression pattern of one or more families of transposable elements in a sample comprising determining expression of one or more families of transposable elements.

As used herein a “sample” can be from any organism and can be, but is not limited to, peripheral blood, plasma, urine, saliva, gastric secretion, feces, bone marrow specimens, primary tumors, metastatic tissue, embedded tissue sections, frozen tissue sections, cell preparations, cytological preparations, exfoliate samples (e.g., sputum), fine needle aspirations, amino cells, fresh tissue, dry tissue, and cultured cells or tissue. It is further contemplated that the biological sample of this invention can also be whole cells or cell organelles (e.g., nuclei). The sample can be unfixed or fixed according to standard protocols widely available in the art and can also be embedded in a suitable medium for preparation of the sample. For example, the sample can be embedded in paraffin or other suitable medium (e.g., epoxy or acrylamide) to facilitate preparation of the biological specimen for the detection methods of this invention.

The sample can be from a subject or a patient. As utilized herein, the “subject” or “patient” of the methods described herein can be any animal. In a preferred embodiment, the animal of the present invention is a human. In addition, determination of expression patterns is also contemplated for non-human animals which can include, but are not limited to, cats, dogs, birds, horses, cows, goats, sheep, guinea pigs, hamsters, gerbils, mice and rabbits.

The sample can comprise a cell or cells selected from the group consisting of: a carcinoma cell, a fibroma cell, a sarcoma cell, a teratoma cell, a blastoma cell, a breast tumor cell of epithelial origin, an ovarian tumor cell of epithelial, stromal or germ cell origin, mixed cell types from a tumor or any other cancer cell. The present invention also provides for the analysis of a sample comprising a normal cell or normal cells from a particular tissue. The patterns obtained from normal cells can be compared to the expression patterns for cancerous cells in order to access the differences between normal and cancerous cells.

The term “cancer,” when used herein refers to or describes the physiological condition, preferably in a mammalian subject, that is typically characterized by unregulated cell growth. Examples of cancer include but are not limited to ras-induced cancers, colorectal cancer, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, prostrate carcinoma, rhabdomyosarcoma, colon carcinoma, ovarian cancer and head and neck cancer. While the term “cancer” as used herein is not limited to any one specific form of the disease, it is believed that the methods of the invention will be particularly effective for cancers which are found to be accompanied by changes in transposable element expression, transposable element methylation and/or changes in chromatin status of transposable elements.

There are numerous transposable element families that can be analyzed by the methods of the present invention, including, but not limited to, retroelement families and DNA element families. The retroelement families that can be analyzed utilizing the methods of this invention include but are not limited to, endogenous retroviruses (ERVs), short interspersed nuclear elements (SINEs), long interspersed nuclear elements (LINEs), the vertebrate long terminal repeat (LTR)-containing elements, and the poly(A) retrotransposons. The DNA element families that can be analyzed by the methods of the present invention include, but are not limited to the Mariner/Tci superfamily (e.g. human Mariner, Tigger, Marna, Golem, Zombi), hAT (hobo/Activator/Tam3) superfamily, TTAA superfamily (e.g. Looper), MITEs (e.g. MER85), MuDR superfamily (e.g. Ricksha), T2-family (E.G. Kanga 2) and others. Any combination of retroelement families and the members of these retroelement families can be analyzed by the methods of the present invention to determine a pattern of expression, a retroelement methylation pattern and/or a retroelement chromatin status pattern. For example, one of skill in the art could analyze the expression of ERVs as well as the expression of SINEs or one of skill in the art could analyze the expression of SINEs, LINEs and ERVs. As stated above, any combination of families and members of transposable element families may be analyzed to provide an expression pattern, chromatin status pattern and/or a methylation pattern. Therefore, combinations of retroelement families and DNA element families can also be also analyzed by the methods of the present invention. A publicly available database, RepBase Update, contains consensus sequences of genomic repeats from different organisms that can be utilized to design the oligonucleotides utilized in the methods of the present invention. This database can be accessed at www.girinst.org. This database was utilized to identify consensus sequences for numerous retroelements which were then used to design oligonucleotide probes for the microarrays of the present invention.

Files were obtained from RepBase Update containing human-specific repeats (consensus sequences for transposon families). Selected RepBase files were then input into the OligoArray program, a publicly available software tool for microarray oligo-design at http://berry.engin.umich.edu/oligoarray and the design algorithm was run. The BLAST algorithm at http://www.ncbi.nlm.nih.gov/BLAST/ (Altschul S F, Gish W, Miller W, Myers E W, Lipman D J Basic local alignment search tool. in J Mol Biol Oct. 5, 1990;215(3):403-10)) was then utilized to verify compatibility of oligonucleotides in the OligoArray output file with transposon sequences in the human genome sequence (http://www.ncbi.nlm.nih.gov/genome/guide/human/). Selection of appropriate oligonucleotides was based on several criteria such as, the quality of match/specificity, technical parameters and the broad representation of transposable element families. Utilizing this approach, numerous oligonucleotides were designed based on these consensus sequences. The identifiers of retroelement consensus sequences and their corresponding oligonucleotide sequences which can utilized in the methods described herein, are listed in Table 1. Similar analyses can be performed to obtain consensus sequences for non-retroelement transposable element sequences.

TABLE 1

| | | |
|---|---|---|
| FLA | GAGTTCGAGACCAGCCTGGGCAACATAGCGAGACCCCGTCTCTAAAAAAA | SEQ ID NO: 1 |
| FLAM_A | GGAGTTCGAGACCAGCCTGGGCAACATAGCGAGACCCCGTCTCTAAAAAA | SEQ ID NO: 2 |
| FLAM_C | GGAGTTCGAGACCAGCCTGGGCAACATAGCGAGACCCCGTCTCTAAAAAA | SEQ ID NO: 3 |
| AluJo | GAGGCAGGAGGATCGCTTGAGCCCAGGAGTTCGAGGCTGCAGTGAGCTAT | SEQ ID NO: 4 |
| AluJb | GGAGTTCGAGACCAGCCTGGGCAACATGGTGAAACCCCGTCTCTACAAAA | SEQ ID NO: 5 |
| AluSc | TCACGAGGTCAAGAGATCGAGACCATCGTGGCCAACATGGTGAAACCCCG | SEQ ID NO: 6 |
| AluSg | CCAACATGGTGAAACCCCGTCTCTACTAAAAATACAAAAATTAGCCGGGC | SEQ ID NO: 7 |
| AluSp | CCAGCCTGACCAACATGGAGAAACCCCGTCTCTACTAAAAATACAAAAAT | SEQ ID NO: 8 |
| AluSq | CAACATGGTGAAACCCCGTCTCTACTAAAAATACAAAAATTAGCCGGGCG | SEQ ID NO: 9 |
| AluSx | CCAACATGGTGAAACCCCGTCTCTACTAAAAATACAAAAATTAGCCGGGC | SEQ ID NO: 10 |
| AluSz | CCAACATGGTGAAACCCCGTCTCTACTAAAAATACAAAAATTAGCCGGGC | SEQ ID NO: 11 |
| AluY | GAGATCGAGACCATCCTGGCTAACACGGTGAAACCCCGTCTCTACTAAAA | SEQ ID NO: 12 |
| AluYa5 | CGGGCGGATCACGAGGTCAGGAGATCGAGACCATCCCGGCTAAAACGGTG | SEQ ID NO: 13 |
| AluYa8 | GAAACCCCGTCTCTACTAAAACTACAAAAAATAGCCGGGCGTAGTGGCGG | SEQ ID NO: 14 |
| AluYb8 | AGACCATCCTGGCTAACAAGGTGAAACCCCGTCTCTACTAAAAATACAAA | SEQ ID NO: 15 |
| AluYb9 | AGACCATCCTGGCTAACAAGGTGAAACCCCGTCTCTACTAAAAATACAAA | SEQ ID NO: 16 |
| AluYc1 | GAGATCGAGACCATCCTGGCTAACACGGTGAAACCCCGTCTCTACTAAAA | SEQ ID NO: 17 |
| AluYc2 | GAGATCGAGACCATCCTGGCTAACAAGGTGAAACCCCGTCTCTACTAAAA | SEQ ID NO: 18 |
| AluYd3a1 | CGCCTGTAGTCCCAGCTACTCGGAGAGGCTGAGGCAGGAGAATGGCGTGA | SEQ ID NO: 19 |
| AluYe | ACCATCCTGGCTAACACGGTGAAACCCCGTCTCTACTAAAAATACAAAAA | SEQ ID NO: 20 |
| LTR26B | ATGGATTTGAGGTTTCCTCCCATCTCCTCATTGGGCGGCCCTACGATTAA | SEQ ID NO: 21 |
| LTR26C | ACGGATTTGAGGTTTCCTCCCATCTCCTCATTCGGCAGCCCTACGATTAA | SEQ ID NO: 22 |
| LTR26D | GGCGTATTGACTTGCTGTGTGCATCGGGCAATGAACCTATTACGGTTACA | SEQ ID NO: 23 |
| AluYa1 | GAGATCGAGACCATCCTGGCTAACACGGTGAAACCCCGTCTCTACTAAAA | SEQ ID NO: 24 |
| AluYa4 | CGGGCGGATCACGAGGTCAGGAGATCGAGACCATCCCGGCTAAAACGGTG | SEQ ID NO: 25 |
| AluYb3a1 | GAGATCGAGACCATCCTGGCTAACACGGTGAAACCCCGTCTCTACTAAAA | SEQ ID NO: 26 |
| AluYb3a2 | GAGATCGAGACCATCCTGGCTAACACGGTGAAACCCCGTCTCTACTAAAA | SEQ ID NO: 27 |
| AluYe5 | ACCATCCTGGCTAACACGGTGAAACCCCGTCTCTACTAAAAATACAAAAA | SEQ ID NO: 28 |
| AluYf1 | GAGATCGAGACCATCCTGGCTAACACGGTGAAACCCCGTCTCTACTAAAA | SEQ ID NO: 29 |
| AluYg6 | GAGATCGAGACCATCCTGGCTAACACGGTGAAACCCCGTCTCTACTAAAA | SEQ ID NO: 30 |
| AluYh9 | GAGATCGAGACCATCCTGGCTAACGCGGTGAAACCCCGCCTCTACTAAAA | SEQ ID NO: 31 |
| AluYl6 | AGATCGAGACCATCCTGGCTAACACGGTGAAACCCCGTCTCTACTAAAAA | SEQ ID NO: 32 |
| AluYbc3a | AGATCGAGACCATCCTGGCTAACACGGTGAAACCCCGTCTCTACTAAAAA | SEQ ID NO: 33 |
| AluYe2 | GACCATCCTGGCTAACACGGTGAAACCCCGTCTCTACTAAAAATACAAAA | SEQ ID NO: 34 |
| AluYf2 | GATCGAGACCATCCTGGCTAACACAGTGAAACCCCGTCTCTACTAAAAAA | SEQ ID NO: 35 |
| ALU | GAGGCAGGAGGATCGCTTGAGCCCAGGAGTTCGAGGCTGCAGTGAGCTAT | SEQ ID NO: 36 |
| MIR | GGCTCTGCCACTTACTAGCTGTGTGACCTTGGGCAAGTTACTTAACCTCT | SEQ ID NO: 37 |
| L1PA2 | ATCACATGGACACAGGAAGGGGAATATCACACTCTGGGGACTGTGGTGGG | SEQ ID NO: 38 |
| L1PA7 | CCTGTCGGGGGGTGGGGGGCTAGGGGAGGGATAGCATTAGGAGAAATACC | SEQ ID NO: 39 |
| L1PA11 | TGGGCTTAATACCTAGGTGATGGGATGATCTGTGCAGCAAACCACCATGG | SEQ ID NO: 40 |
| L1PA15 | TCGGGTACTATGCTTATTACCTGGGTGACGAAATAATCTGTACACCAAAC | SEQ ID NO: 41 |
| L1PB1 | ATCTCAGAAATCACCACTAAAGAACTTATTCATGTAACCAAACACCACCT | SEQ ID NO: 42 |
| L1PB3 | AAGTGGGAGCTAAGCTATGGGTACGCAAAGGCATACAGAGTGGTATAATG | SEQ ID NO: 43 |
| L1MA2 | GGGAAGGGTAGTGGGGGGTTGGTGGGGAGGTGGGGATGGTTAATGGGTAC | SEQ ID NO: 44 |
| L1MA5 | ATAGGGAGAGGTTGGTTAATGGATACAAAATTACAGCTAGATAGGAGGAA | SEQ ID NO: 45 |
| L1MA9 | AGATCTTAAGTGTTCTCACCACACACAAAAAAATGGTAACTATGTGAGGT | SEQ ID NO: 46 |
| THE1B | CTGCACAWGCTCTCTTGCCTGCCGCCATGTAAGACGTGMCTTTGCTCCTC | SEQ ID NO: 47 |
| MSTA | TCCCCTTGGTGCTGTCCTCGTGATAGTGAGTGAGTTCTCGTGAGATCTGG | SEQ ID NO: 48 |
| MSTC | GATTAATGGATTAATGGGTTATCATGGGAGTGGGACTGGTGGCTTTATAA | SEQ ID NO: 49 |
| MLT1A | TGAGGACACAGTGAGAAGGCGCCGTCTACGAACCAGGGAATGAGCCCTCA | SEQ ID NO: 50 |
| MLT1B | GGAGAAGACGGCCATCTACAAGCCAAGGAGAGAGGCCTCAGAAGAAACCA | SEQ ID NO: 51 |
| MLT1C | CCAGCAAACCACCAGAAGCTAGGGGAGAGGCATGGAACAGATTCTCCCTC | SEQ ID NO: 52 |
| MLT1D | GGTCAGAGTCAGAGAAGGAGATGTGACGACGGAAGCAGAGGTCGGAGTGA | SEQ ID NO: 53 |
| MLT1E | GATTCCGTCTTGNCGNCANTCTTGCTGAGAGNCTCTCTTGCTGGCTTTGA | SEQ ID NO: 54 |
| MLT1F | TGTAGTCCCCTCCCACATTGAATAGGGCTGACCTGTGTGACCAATAGAAT | SEQ ID NO: 55 |
| THE1BR | CAAGAGGTGACTTGGGTGCTGTTAAAGGCATTCAGTTTTAAAAGGGAAGC | SEQ ID NO: 56 |
| MSTAR | TCTTTTTGATTTTACAGGCTCATAGGTGGAAGGAACTTGCCTTGTCTCAG | SEQ ID NO: 57 |
| MLT1R | AGCCTGATCATGTAACAGAAANNNCAATAGCGTTCTCTGGAAAGAANACC | SEQ ID NO: 58 |
| MLT2A1 | GGGTGTTGCCAAAGGAGGTTAACATTGGACTCAGTGGGCTGGGGAGAGGC | SEQ ID NO: 59 |
| MLT2B2 | TTCCAGATGAGATTAGCATTTGAATCAGCGGACTGAGTAAAGAAGATTGC | SEQ ID NO: 60 |
| MLT2C2 | CTCAAGACTGCAACGTGGAAATCCTGCTGNTTTWCCAGCCTCCAAGCCTT | SEQ ID NO: 61 |
| MLT2D | GGCTAGGCTATGGTGTGCAGACGTTTGGTCAAACATTAGTCTGGGTGTTT | SEQ ID NO: 62 |
| LTR2 | CAATGCTCCCAGCTGATTAAAGCCTCTTCCTTCATAGAACCGGTGTCTAA | SEQ ID NO: 63 |
| LTR3 | GCAAGGAGCCCCCTGACCCCTTCTTCCAAACATACTCTTTTGTCTTTGTC | SEQ ID NO: 64 |
| LTR4 | ATCCTCCTGTCCCACCCATTGGTCTCTCCTGTCCCTTGATTCCTGCAACA | SEQ ID NO: 65 |
| LTR5 | ACTCAGAGGCTGGTGGGATCCTCCATATGCTGAACGTTGGTTCCCCGGGC | SEQ ID NO: 66 |
| LTR11 | AACTCCGTCACTGTAATCCCAATGTAAAGCAAGAATTCCAAACCAGGAAA | SEQ ID NO: 67 |
| LTR12 | GCTTCATTCTTGAAGTCAGCGAGACCAAGAACCCACCGGAAGGAACCAAT | SEQ ID NO: 68 |
| LTR13 | CTTGTGTCTTTATTTCTACACTCTCTCGTCTCCGCACACGGGGAGAAAAA | SEQ ID NO: 69 |
| MER1A | AAGCTTCATCTGTAKTTACAGCCGCTCCCCATCACTCGCATTACCGCCTG | SEQ ID NO: 70 |
| MER1B | TGATCTGAGGTGGAACAGTTTCATCCCGAAACCATCCCCGCCCCCCGGTC | SEQ ID NO: 71 |
| MER2 | AAAATCCACGGATGCTCAAGTCCCTGATATAAAATGGCGTAGTATTTGCA | SEQ ID NO: 72 |
| MER3 | ATGTGGCTAYTGAGCACTTGAAATGTGGYTAGTGCGACTGAGGAACTGAA | SEQ ID NO: 73 |
| MER4A | GGACCTCAAGATCTTTACCCTAAAACAGTTCTGYTGAMYTTCACCTTGGC | SEQ ID NO: 74 |
| MER4B | TTGGTCTCCGCAACCCCTTATNTCATAACCCGGACATTCCTTTCCATTGA | SEQ ID NO: 75 |
| MER4C | CCTCCCTCTTTCCCCTCCAGCCCGCTTTTCCCCTTTAAATATTGAAGCCC | SEQ ID NO: 76 |
| MER5A | GTCCCCGGACCAGCAGCATCAGCATCACCTGGGAACTTGTTAGAAATGCA | SEQ ID NO: 77 |
| MER5B | TCAGTATTTTTTAAARCTCYYCAGGTGATTCCAATGTGCAGCCAAGGTTG | SEQ ID NO: 78 |
| MER6 | AAGTCGCAGTTTCGAAGAACCTATCGACGACGTTAAGTGAGGACTTACTG | SEQ ID NO: 79 |
| MER8 | AAAAATCCGCGTATAAGTGGACCCACGCAGTTCAAACCCGTGTTGTTCAA | SEQ ID NO: 80 |
| MER9 | GCTGTGAGACCCCTGATTTCCCACTTCACACCTCTATATTTCTGTGTGTG | SEQ ID NO: 81 |
| MER11A | TGATTTTGCCCTTGTCCTGTTTCCTCAGAAGCATGTGATCTTTGTTCTCC | SEQ ID NO: 82 |
| MER11B | ACTTGCTGGTTTTTGCGGCTTGTGGGGCATCACGGAACCTACCGACATGT | SEQ ID NO: 83 |
| MER20 | CCCCACAACAAAGAATTATCCGGCCCAAAATGTCGATAGTGCVAAGGTTG | SEQ ID NO: 84 |
| MER21 | SAGCAGAGGRAAAACATGGTTTGAGAGAGGTTTTYCTGMAAYAGRAGGGC | SEQ ID NO: 85 |
| MER21B | CGGTCAGAAGCACAGGTNACAACCTGGNGCTTGCGACTGGCATCTGAAGT | SEQ ID NO: 86 |
| MER22 | TGAGTCTCCCCAAAAGTGGAGCCCTTGTGATGACGAGCACAGGTCCGCCT | SEQ ID NO: 87 |
| MER28 | AAGACGANGAGGATGAAGACCTTTATGATGATCCACTTCCACTTAATGAA | SEQ ID NO: 88 |
| MER30 | TTTTAAGAAAGTTTACGAATTTGTGTTGGGCCGCATTCAAAGCCATCCTG | SEQ ID NO: 89 |
| MER35 | GATGAAAAGGGGATCCTGTGCAGAAACCACACTACCCATCAGAGAAGCAA | SEQ ID NO: 90 |
| MER39 | GGCAGGTCATAGAAACTAGAACTCCTCTCCCCCAAAGCAAGCCATAAAAC | SEQ ID NO: 91 |
| MER44A | AGGGTTCGGTACTATCCGCGGTTTCAGGCATCCACTGGGGGTCTTGGAAC | SEQ ID NO: 92 |
| MER44C | CGCACCTCAAACTGCAAAAGTTACGGCCACAGTGCGTGATAAGTGCTTAG | SEQ ID NO: 93 |
| MER45 | GAAATTCTTAATAATTTTTGAACAAGGGGCCCCGCATTTTCATTTTGCAC | SEQ ID NO: 94 |
| MER48 | TGTTGTTGTGGACGCGCTCTCGGGGTTSCAACCGAYACAAGARCCTTACA | SEQ ID NO: 95 |
| LOR1 | TCTTCCTTGGCAATAMTYRTTGTCTCAGTGATTGGCTTTCTGTGCAGTGA | SEQ ID NO: 96 |
| SVA | GGGGAAAGGTGGGGAAAAGATTGAGAAATCGGATGGTTGCCGTGTCTGTG | SEQ ID NO: 97 |
| ALR | GTGGAGATTTCAGCCGCTTTGAGGTCAATGGTAGAATAGGAAATATCTTC | SEQ ID NO: 98 |
| MSR1 | GGAGTCAAGACCGCCCAGCCCCTCCTCCCTCAGACTCATGAGTCCAGACC | SEQ ID NO: 99 |
| TAR1 | ACTCATGGAGGGTTAGGGTTCAGGTTCGGGTTCGGGTTCGGGTTCGGGTT | SEQ ID NO: 100 |
| CER | GGTTCTGAGTGTTTGTCCCTCACATAGGATTCCAGAACACTGCTGCTGGG | SEQ ID NO: 101 |
| BSR | TCACAATGCCCCTGTAGGCAGAGCCTAGACAAGAGTTACATCACCTGGGT | SEQ ID NO: 102 |
| HSATII | GGGTCCATTCGATGATGATCACACTGGATTTCATTCCATAATTCTATTCG | SEQ ID NO: 103 |
| HSATI | CCACTGTCTGTGCTGTGTCTTTCAAAGGTCAGAAGAGATTGNACCTTTGT | SEQ ID NO: 104 |
| R66 | TGCRTTTACAAACCTTTAGCTAGACACAGAGCGCTGATTGGTGCGTTTTT | SEQ ID NO: 105 |
| SN5 | CCTGACTCCTGAGTCACGTTACTGTCCCACTATACGTTAAGAGGAGGGAA | SEQ ID NO: 106 |
| HIR | AATATCAGGAACACCGGCATGTGCACTTAGGACCATGTTTTAATTTTTCA | SEQ ID NO: 107 |
| GGAAT | GGAATGGAATGGAATGGAATGGAATGGAATGGAATGGAATGGAATGGAAT | SEQ ID NO: 108 |
| KER | GGATGAGGCAGGAAAGACAGCTGAGGGTCAGAACCCAGGCAGGTCCAATG | SEQ ID NO: 109 |
| TIGGER1 | ACTCGCTGAAGGCTCAGATGATCGTTAGCATTTTTTAGCAATAAAGTATT | SEQ ID NO: 110 |
| TIGGER2 | TAAAGTTACACCGAGTGTGCCTGCCTCTCCTGCCTCCCCTTCCACCTCCT | SEQ ID NO: 111 |
| GSAT | GGGACTCAGGAGGATGTTGAGGGAGACAGAGGGGTGAAGCGTTGAGACGA | SEQ ID NO: 112 |
| GSATX | CAGGCGGCCAGNCTTTCAGGGGGAGGATGAAGTAGGCCTGGGACAAAAGC | SEQ ID NO: 113 |
| HERVL | AGGACTCTACTTCTAATAGTATGGAGAACACTGATAGTCCTTGGCATGAA | SEQ ID NO: 114 |
| HERVK | CCCTGTCACTTGGGTTAAGACCATTGGAAGTACATCGATTATAAATCTCA | SEQ ID NO: 115 |
| HERVR | AACCCAACAGTATCAGGTGCTCAGAACCGATGAAGAAGCTCAAGATTGAG | SEQ ID NO: 116 |
| HRES1 | TGGTTAATGTGTAACAAGGAGGCAGTAGGCCCCAGGTGTCCAGCCAGAGG | SEQ ID NO: 117 |
| HERVE | AAAAGTGAGGACGAGAGTAAGAACTCCCACTAAAAGTGAAAATTCTCAAA | SEQ ID NO: 118 |
| HERVH | CATACCACCCCCCAAAAATTTTCACTGCCCCAACACTTCAACACTATTTT | SEQ ID NO: 119 |
| HERVI | TTGTAGGATGCTGTGTCATACCCTGTGCCCTAGGATTAATACAAAAGCTC | SEQ ID NO: 120 |
| LTR14 | GCCTCCACTCTTTATGAACTCTTAACCTGTCTCTTCTCATTCCTTTGTCA | SEQ ID NO: 121 |
| HERVKC4 | CCGGATCATTCACAGAGTTCAATTCAATTAACAGTTTAAGCCCCCAAAAA | SEQ ID NO: 122 |
| MER4I | AGAGATCAGACGAAACCTGAGACCAGAGACTCATTTTCTTCTAAAATGCT | SEQ ID NO: 123 |
| MER49 | ACATGCATGTTTGTTCAATACGCATGCGTCAGGACCACCTTCATGAATAT | SEQ ID NO: 124 |
| MER4D | CAACCCCCCTTATCTTAACTCAAGCTGACTTCAACTCTTCAGGCAGAGCT | SEQ ID NO: 125 |
| MER39B | GCCCTCCTGTCTCTCAGTCCCA1TCTCCCCCGAGGCTAGCCATAGAAACT | SEQ ID NO: 126 |
| IN25 | TCTTGGAGAAGGGATCCTTGTTCCCCNCTGGCNCTGGTANNCCACTGCAG | SEQ ID NO: 127 |
| MER61 | AAGCCTAAWTTTTCGTGGCCGTGTGACAAGGACCCCGTCTTTAGCTGAAC | SEQ ID NO: 128 |
| HERV3 | CAACCCTTGCCAAATGAAGAGAACTGGCTTCNCATGAAGAATTAANTAGT | SEQ ID NO: 129 |
| HERV9 | GCACAGAGCGATACAACTAATACCCCTACTTATAGGGTTAGGAATGGCTA | SEQ ID NO: 130 |
| HERVS71 | AAACTGGACTAATGTCCTTGTCCCAACAGGTAGATGCTGATTTAAATAAC | SEQ ID NO: 131 |
| HSMAR1 | CACTTCTTCAAGCATCTCGACAACTTTTTGCAGGGAAAACGCTTCCACAA | SEQ ID NO: 132 |
| HSMAR2 | TGGTATCATCGCTTACAAAAGTGTCTTGAACTTGATGGAGCTTATGTTGA | SEQ ID NO: 133 |
| L1 | AAACAACCCCATCAAAAAGTGGGCAAAGGATATGAACAGACAGTTCTCAA | SEQ ID NO: 134 |
| L1MA10 | GTGATGGTTTCACGGGTGTATGCATATGTCCAAACTCATCAAATTGTATA | SEQ ID NO: 135 |
| L1MB3 | TCAGTTTGGGAAGATGAAAAAGTTCTGGAGATGGATGGTGGTGATGGTTG | SEQ ID NO: 136 |
| L1MB7 | AGATAGTGGTGATGGTTGCACAACTCTGTGAATATACTAAAAACCACTGA | SEQ ID NO: 137 |
| L1MC2 | ATGTTAATAATAGGGGAAACTGTGTGNGGGNGGGGTGAGGGGGTATATGG | SEQ ID NO: 138 |
| L1MC3 | CTGTTGGAGTGGGAGGTTACAGATAAGCAAGGGGAGGAGGCTAGAATGAT | SEQ ID NO: 139 |
| L1MC4 | TATTTAGGGGTAANGGGGCATCATGTCTGCAACTTACTCTCAAATGGTTC | SEQ ID NO: 140 |
| L1MD1 | GCAGGAGGGAAGTGGGTGTGGCTATAAAAGGGCAACATGAGGGATCCTTG | SEQ ID NO: 141 |
| L1MD2 | GNGNGGGGGAAGGGAGGTGGGTGTGGCTATAAAAGGGCAGCACGAGGGAT | SEQ ID NO: 142 |
| L1ME2 | AGTGGTTGCCTCTGGGGAGGGTGANTGACTGGAAAGGGGCATGAGGGAAC | SEQ ID NO: 143 |
| L1ME3A | GGCAAAACTAATCTATGSTGTTAGAAGTCAGGATAGTGGTTACCCTTGGG | SEQ ID NO: 144 |
| LSAU | GGTGTTGGGAGAGCCTCAGCCGGAATTTCGTGGACGGACAAGGGCACAGA | SEQ ID NO: 145 |
| LTR1 | CTAGAGGTTTGAGCAGCGGGGCACTGAAGAAGCGAGCCACACCCCCATCG | SEQ ID NO: 146 |
| LTR15 | ATCCTCCTCAACCCCATCGGTCTCTCTGATTCCTAAATCATCCCCAAACA | SEQ ID NO: 147 |
| LTR8 | TTTCTCTATTGCAATTCCCCTGTCTTGATGAATCGGCTCTGTCTAGGCAG | SEQ ID NO: 148 |
| LTR9 | TAAACTCCTCGTGTGTGTCCGTGTCCTAAATTTTCCTGGCGCGNGACGAC | SEQ ID NO: 149 |
| MER31 | CCTGTACCTATCGCAATGGTCCTGAATAAAGTCTGCCTTACCGTGCTTTA | SEQ ID NO: 150 |
| MER34 | GCCCAAACCCCTTTGTCTTGTCACGTTTTCACAATTTACTACTCTTTGTC | SEQ ID NO: 151 |
| MER41A | GCAACGTCAGGAAGTTACCCTATATGGTCTAAAAAGGGGAGGGATGAATA | SEQ ID NO: 152 |
| MER41B | TGCCATGGCAACGTCAGGAAGTTACCCTATATGGTCTAAAAAGGGGAGGA | SEQ ID NO: 153 |
| MER41C | TAGCAGAGCACATCTCCCCCGTAATGTTCTTTGGCTTTGTTATCCTATAT | SEQ ID NO: 154 |
| MER50 | TGGCCCTCTICCAAGTGTACTTCGCTTCC1TTCG1TCCTGCTCTAAAACT | SEQ ID NO: 155 |
| MER63A | TTCAAGCTACCAACGTGATGTCACTGAATGSGGAGTTGGGAAAAGATATA | SEQ ID NO: 156 |
| MER63B | ATGTCACTGAATGSGGAGTTGGGAAGAGATGCACAGTAGCACACYATTAT | SEQ ID NO: 157 |
| MER63C | ACAATGTAACGGCTACAGACACGACACACTTTTAAGTTTAATCTGCATTA | SEQ ID NO: 158 |
| MER65A | GAATATGCACATAGTTTACTATGGCACGCGTATTCCCATTGCAATGCTCT | SEQ ID NO: 159 |
| MER65B | ACATTTGCCTGACAACTGTCTCACRAACCTAGCTACTGCAAGAGCCTACT | SEQ ID NO: 160 |
| MER66A | AGACTAGCTGAAACAGGGCCAGGGCAAAAGCACCTCTCCATAAGACACAC | SEQ ID NO: 161 |
| MER66B | CTTGAACACCAGACCAAATTGAAGACTAGCTGAAACAGGGCCAGGGCAAA | SEQ ID NO: 162 |
| MER67A | GCCTCAACCTCGGCCTATAAAGACTTGAACAAACACTAACATAGTTTCTA | SEQ ID NO: 163 |
| MER67B | CACAGAACAACTCCATCCAAACCCCTGCACTAAGAGACTTGACCAAACTC | SEQ ID NO: 164 |
| MER67C | TCTTGAGAACATGTATGTAATGGGCTGTATCTGCTCGGCTATATAAAAGG | SEQ ID NO: 165 |
| MER68A | AACCCTGGGCACTGAGTCTCTAATGAGCTTCCCTGGTAGACAACATTTCA | SEQ ID NO: 166 |
| MER68B | TTCCCTTTGCTGATCTTGCCGTGTATCCTTACNRTGTCGCTGTAATAAAT | SEQ ID NO: 167 |
| MER69A | CCCCCAAATTGTATAAGCTTCAGGCCCCACAAAACCTGGATCTGCCCCTG | SEQ ID NO: 168 |
| MER69B | TTACAAAATCATTGTCATATGAAGAGGCGATCAAAGAGTATGCAGCCAAA | SEQ ID NO: 169 |
| MER70A | TGTTCTGTCTCACCGGACTCAGACAAGTTGGTAACCAGTGCACAGTGAAC | SEQ ID NO: 170 |
| MER70B | TCNGACCCCTATTCCTGGTGG1TGGCATAGTGATGATCTTTGCTATTCTC | SEQ ID NO: 171 |
| MER72 | GGCATGAAGCTCAATTGCACATGTGCATG1TTCTCCTITCATAAATATTC | SEQ ID NO: 172 |
| MER73 | GGTGACGGGGTACGACTGGGTTTCAAACAACTTATGTCAGGCCTAAAAAT | SEQ ID NO: 173 |
| MER74 | GGGGGTATGGGCTCTGGATTGGTTGGTTTGCATATGAAAGGCGCGCTCCC | SEQ ID NO: 174 |
| MER75 | TGGCCGAAGATTCA1TTGATGAATCCGATTTTTCCGAAATAGACGATTCT | SEQ ID NO: 175 |
| MER76 | TGTTGCCTTAATCGGCTNCTCTGACACCCGGCAGCTCAGCTCTCTCTCCA | SEQ ID NO: 176 |
| MER77 | GGTGAGCTTCCCTGGTTGGCAATACTCTNTGCATGTTGTCACACATCGTT | SEQ ID NO: 177 |
| MER80 | CCATAGGCTTCACCAGACTGCCAAAGGGGCCCATGGCACAAAAAAGGTTA | SEQ ID NO: 178 |
| MER82 | NTGCAAATGACCGNGAAAGTGCTNCAAGTATTGATTTTGGGGTTACAAAT | SEQ ID NO: 179 |
| MLT1G | CACAAATTCTTTGACACTCTTCCCATCGAGGAGTGGGGTCCGTNTCCTCT | SEQ ID NO: 180 |
| PABL_A | AATAAAAACTCTCTTCCTCCCCAGTTCATCTGCATCTCGTTATTGGGCCA | SEQ ID NO: 181 |
| PABL_B | CCAGTTCATCTGCATCTCGTTATTGGGCCACGAGAATAAGCAGCCCGACC | SEQ ID NO: 182 |
| MER57I | GCAGTTATGGGGGATACTCGGCTC1TTGCACATTTGGATNAGAGAAGCAT | SEQ ID NO: 183 |
| MER65I | CCTGGATAAATTCCCCTGGGGAACTTGAGGCCCCATATACACGAAATTAC | SEQ ID NO: 184 |
| MER41I | TTTGTTGGGAACTOAGTTACAAATAACCCTCACCATACCAGTACTTTCTG | SEQ ID NO: 185 |
| PTR5 | CATGCTTAAGGAGCCCTTCAGCCTGCCACTGCACTGTGGGAACACTGGCC | SEQ ID NO: 186 |
| L1M2_5 | CGCCTCCTCCACAAAGAAGAACCAAAATAGCGAGTAGATAATCACACTTT | SEQ ID NO: 187 |
| LTR10A | TGCTCCATCTGCGAGACGCACCCTTCTATAGAAGTAAAATTGCCTTGCTG | SEQ ID NO: 188 |
| LTR10B | GCTGAGAGACCCTTTGTCCTTTGGCTCAGTGTTGGTTCTTCTTTGCAGCA | SEQ ID NO: 189 |
| LTR10C | CAGTGTACTCTCATGGCAAAACTGCTGGTGAGTGTACCCTTTCTGCAGAA | SEQ ID NO: 190 |
| LTR16A | CTGCATTGCAGCCCAACTTCTCCCTCTGCCCAATCCTGCTTCCTTCCCTT | SEQ ID NO: 191 |
| LTR17 | CCAAGAACCCCAGGTCAGAGAACACGAGGCTTGCCACCATCTTGGAAGTG | SEQ ID NO: 192 |
| MER41D | GCACGTAGGCACAGCTTAGTTTAGTCTTTACATAGACAAGACTCCTATAT | SEQ ID NO: 193 |
| MER51A | TCCGCAACCAATCAGACGTTTGCATAGGAGTGTAACTTTGTAACTTCACT | SEQ ID NO: 194 |
| MER51B | CTTTACTTCGTCCTCTTCATTTACATAGGGCGTACCCCAAGTAACCAATG | SEQ ID NO: 195 |
| MER57A | ATCTTCTACCACATGGCTGCACTGGAGTCTCTGAACCTACTCTGGTTCTG | SEQ ID NO: 196 |
| MER57B | TATAAATTTGTTCCGACCACGAGGCATCCCTGGAGTCTCTCTGAATCTGC | SEQ ID NO: 197 |
| MER65C | CAACCCTGGCTGCTGAAACTGCCTGTTGTAACCTGAAACCAGTTTTATCT | SEQ ID NO: 198 |
| MER83 | TCTGCAGCCCAAGAACCATCCTATAAAATCTCCAGCAAGCCTTTGTCTCC | SEQ ID NO: 199 |
| MER84 | CATAAATGCTCCTAAGGAAAAATCCACCGCGGCGCGCTCAGTCCTCTCTT | SEQ ID NO: 200 |
| HERV16 | TTGACTATGATGTGTAGGAGGGGTAGGGCTGCTTTAGTAAAATGAGTAAG | SEQ ID NO: 201 |
| HERV17 | GAAGGCACCCCTCCCGAGGAAATCTCAACTGCACGACCCCTACTACGCCC | SEQ ID NO: 202 |
| PMER1 | GTTCTCAACCTTCCTAATGCCGCGGCCCTTTAATACAGTTCCTGTGGGTC | SEQ ID NO: 203 |
| MER54 | TGAAAGATACACTGTAAACACCCACAACCAMCTTCCCTGGAGCCCCATCA | SEQ ID NO: 204 |
| LTR18A | TGTACATACGGCTTGCGCCCAGGCTCACTCGCGCCCAGAGAGAGAGTAAA | SEQ ID NO: 205 |
| LTR18B | ATGAGAGAGCTGCTGAATAAAACCATATTTCACCTGCCTACGGCCCCCCG | SEQ ID NO: 206 |
| LTR19A | AGAGAGTGCTCCTGACTGAAATCGGCCAGAAGCCCCTCTCAGGTTTATTC | SEQ ID NO: 207 |
| LTR19B | GACTGKWGAGCCGCTTTTCGTGTTTCTTTCCTCTTTCTTTAATTCTTACA | SEQ ID NO: 208 |
| LTR20 | AATAAATTCTGCTCYACCTCACCCTTCAATGTGTCTGCATGCCTAATTCT | SEQ ID NO: 209 |
| LTR16C | GTAACTNGCTTGATAACGCACCCTTTATTGGCTTCCTTCCCTTCCCTGTC | SEQ ID NO: 210 |
| LTR21A | CTGCTTYCCTTGACTGTKAWGGGGGCAGCCGRCAGGTTAATAAARGCTTG | SEQ ID NO: 211 |
| LTR21B | CAATAAAGCTTGCTTGCCTGACTTTGGGTCTCYTCATCCTTTCTCTCGGC | SEQ ID NO: 212 |
| MER85 | TTGAGCAGTAGGATATAAATAACTCCCACATGCTTAGCGTTCCAATAATG | SEQ ID NO: 213 |
| LTR22 | GTGCYAGCTGNTTAGGGCCAGCWGCWGTKAGAAACCTYYCTTGGWGTSTG | SEQ ID NO: 214 |
| LTR23 | CCTTTAAAAACCACTTGTAACTGCTGCTAATTGGAGTGTATATTCAGGGC | SEQ ID NO: 215 |
| LTR24 | AAACCTTAACTTCTCCACTTTGGAACGCTGACCCCATTCCTTTGGAGTCT | SEQ ID NO: 216 |
| HERV23 | GTCCTGTCCCCCCAACCATGTGAGATAGAGCCATCTGGGAATGAGCTTTA | SEQ ID NO: 217 |
| HERV18 | AGCGGGAATATTAGTGGTGAGTTGTTGCTCCCTGTATTGTTGCTGTGGCC | SEQ ID NO: 218 |
| MER87 | ACTTACTGGCTGTCGWGCGGTGAGCAGTACCAGCTTTGGATTCAGTTACA | SEQ ID NO: 219 |
| MER74A | AATGGCAGTCGTCTCCTGATCTGTTGGCCTTACCATACCTGAATAATAAT | SEQ ID NO: 220 |
| MER74B | CTTTTCAATGGCAGTCGTCTCCTGATCTGTTGGCCTTACCATACCTSAAT | SEQ ID NO: 221 |
| MER88 | AGGGGAACTTGTGGCAGGGACCAGCCTTATCACACTGGTGCACCTGGTCA | SEQ ID NO: 222 |
| MER54B | GAGCCCAGTCTGCTAGGCGGGAGAGATGCCTCTAAGTTCTTATCTCTGGC | SEQ ID NO: 223 |
| MER31A | GGCTCCTGAACCTTCTCCTAGGCCCATCTGTGCACTTCCTTGTAAAATCC | SEQ ID NO: 224 |
| MER31B | GCCCTGTCCTTGGCCTGCWTAGCCCAGTTTTAGCAAGAATCCTGCTAAGT | SEQ ID NO: 225 |
| MER67D | ATCCACCTGCCTTTTGTTTCAGNGGAGTTGAGTTCAANCTCTAACCCCTA | SEQ ID NO: 226 |
| MER31I | GATGATTCAGCTGGTCCTTAATGAACAAAAGGCMACCCAACAAGAAAATG | SEQ ID NO: 227 |
| CHARLIE1 | TTCCACATTGCAACTAACCTTTAAGAAACTACCACTTGTCGAGTTTTGGT | SEQ ID NO: 228 |
| CHARLIE1A | CACCGCAACTAACCTTTAAGAAACTACCACTTGTTGAGTTITGGTGTAGT | SEQ ID NO: 229 |
| CHARLIE1B | CAGTGGAGTTTTCCAGAGGCTACATGACGTGTGATGTCGCAACAGATTGA | SEQ ID NO: 230 |
| CHARLIE2 | TAAAATTCTGTGGGGGAAGTGGAATGGAAATACGAGTTCAAGGAGAAAAA | SEQ ID NO: 231 |
| MER30B | CAATCTTTTGGCTTCCCTGGGCCACATTGGAAGAAGAATTGTCTTGGGCC | SEQ ID NO: 232 |
| MER45B | CCGCATACGAGTTAAATGCTCTTATATTTGCATTTAAAACTGGCATTGCA | SEQ ID NO: 233 |
| MER45C | GCGAGTATCCCCGTGCCCGAGGGAGCGTGACATTAAATAGCAAATAAAAA | SEQ ID NO: 234 |
| LTR25 | CTCTCCGCTGRCAGAGAGCTTTCTTCTTTCACTTATTAAACTTTCACTCC | SEQ ID NO: 235 |
| LTR26 | TCTCAGTGTAATTGGTCTGTTACTGCGCAGTGGGCATATGAACCTGTTGG | SEQ ID NO: 238 |
| HERVK9I | ATCCCGACTCCTGCGAGAAGTAGCTCACCGTGACAAAGCTGCCTTTGCTT | SEQ ID NO: 237 |
| HERVH48I | TCTCTCAAGAATACCCCAAAAATTAAGTTTTTCTTTTTCCAAGGTGCCCA | SEQ ID NO: 238 |
| MER11C | CCTGTGATCTCGCCCTGCCTCCACTTGCCTTGTGATATTCTATTACCYTG | SEQ ID NO: 239 |
| MER11D | TTCATCCCCATGTGACCATCTCACCTCATAATCAAATGACCCTAAATCCC | SEQ ID NO: 240 |
| LTR10D | GGCGACTGGCCAAGGAGAAGCACCCCTCTGCGCAGAAGTAAAATTGCTTT | SEQ ID NO: 241 |
| LTR14A | CCACACTCGCGATGGCCCCCTGGTCCCACTTTCTCTCTCAAACTGTCTTT | SEQ ID NO: 242 |
| LTR14B | TTTGCAGCCTCCATACTTAGCGTTGGCCCCCTGGACCCACTTTCTCTCTC | SEQ ID NO: 243 |
| LTR27 | GTGGGACAAGAACTTGGGAATCAGTGCACAAGCCAGACTTGGCCTGGGAA | SEQ ID NO: 244 |
| LTR28 | ATTGATCCCCACCCTTCACCTATTTTACATATACCCACCCTTTCCTAATT | SEQ ID NO: 245 |
| LTR29 | TTAATCAATCTGCCTTNTGTCAGTGATTTTTCAGCGAACCTTCAGGGGGC | SEQ ID NO: 246 |
| LTR30 | CTTTTTTTCTCTCTTGGTCCGATCCGTGTCTCTCWCTCGCCGCGGGCWGC | SEQ ID NO: 247 |
| LTR31 | TTTCTCTTTTGCAAAACCCATCGTCACAGTGATTGRCTTACTGCGCGCGG | SEQ ID NO: 248 |
| MER61B | ACCCTTTCCTGACTGATTCTCTCTGAATAATGCCCACCTGCGCACTGGGA | SEQ ID NO: 249 |
| MER61C | CCGACCGGCCCCACAAGTGTTTACATCAGATGCTTTTGTGCAGATGAGGG | SEQ ID NO: 250 |
| MER92A | CGCTTGCCCACTGTCYCCTTTCTACTGGTTCTGCTTAYCYCTCCCTATAA | SEQ ID NO: 251 |
| MER92B | TTCTGCCTGAACTTTGAGATGCTTGCAGATCTTATGGTCAGAGCGTTCTC | SEQ ID NO: 252 |
| MER92C | TATCTACCCCTTCCTATAAAAGTCCAAGGCAAAACCACCCTGCCGAGACA | SEQ ID NO: 253 |
| MER93 | GCCCTGGGTTCCTACGTAAGCAAACCGAAACCTAACTCAGNCGTTTCTTA | SEQ ID NO: 254 |
| MLT1H | CACAGATGCATGAGGGAGCCCAGCCGAGACCAGAAGAACCACCCAGCTGA | SEQ ID NO: 255 |
| L1P_MA2 | GAACCCAGAAACAAATCCATACATYTACAGCGAACTCATTTTCGACAAAG | SEQ ID NO: 256 |
| LTR32 | ATGTAAGTCCCCAATAAACCCTATGTCTCATITGCTGGCTCTGGGTCTCT | SEQ ID NO: 257 |
| GOLEM | GCACAACGACGAAATCGCCTAACGACGCATTTCTCAGAACGTATCCCCGT | SEQ ID NO: 258 |
| ZOMBI | TAGTGACACCTTTGCTTTCTGATGGTTCAATGTACACAAACTTTGTTTCA | SEQ ID NO: 259 |
| ZOMBI_A | CGGATTTTCAGATTTGGGATGCTCAACCGGTAAGTATAATGCAAATATTC | SEQ ID NO: 260 |
| ZOMBI_B | NCTGCCAGNCAACNACAGNTTGTGCACCTNGNTGGCARAGANACTGACAC | SEQ ID NO: 261 |
| LTR33 | CGCTGTTGCTAGCCCCGGGGTGCTTCACCATCCCTTGTTGGTTTCCCTTA | SEQ ID NO: 262 |
| L1PA12_5 | AAGTCAGCTTGAAATAAAGACCCTGCACAAAGCCTCGGCCCGGTGAAAAC | SEQ ID NO: 263 |
| L1PA16_5 | GACAGCCANACAATAGACAGCCTGTCAATAGANATAGCCACACAATAATA | SEQ ID NO: 264 |
| L1PBA_5 | AAGAATCTGAACAGCAGCCCTTGAGTCCCAGATCTTCCCTCTGACATAGT | SEQ ID NO: 265 |
| L1PBB_5 | AATCTACCCACCTGCTTTAGCCACARCTGGTKYYTACCCAKGGAYACCTC | SEQ ID NO: 266 |
| L1M3A_5 | AAGAAACATAWTCACATTCAARGGAGTCCCAATATGGCTATCAGCAGATT | SEQ ID NO: 267 |
| L1M3B_5 | AGTGGMAATCTCATCAGCCCAGGGATCTRACAGGAGAAGGTCTTCCTCCC | SEQ ID NO: 268 |
| L1M3C_5 | YACATCMATAGAAAAGGTCTGAGAGAGYCCCAGAATCCCTAGCCAGGCTG | SEQ ID NO: 269 |
| L1M3D_5 | GTCGCGCTACGCTGATANGATTNANCATACCCTANATGCTCGGCGACTGC | SEQ ID NO: 270 |
| L1MB6_5 | CACTCAGTGCGAAAAAGCATTATACCTGGGGGCATTGTIGAAAACAWTTA | SEQ ID NO: 271 |
| L1MCA_5 | TGAAAGTGGACTTGGATTAGTTGTAAATGTATATTGCAAACTCTAGGGCA | SEQ ID NO: 272 |
| L1MCB_5 | CTGACACCTACAGCTACAGCAAACAGTAAACACAGTCTAACTCTTAGCCA | SEQ ID NO: 273 |
| L1MEA_5 | ACCACAGCCACTGGAAAGAGTGGGGAAAATCCCGGAAAGGAGAGAGCCAG | SEQ ID NO: 274 |
| L1MEC_5 | ACAAAAATATCCAGCACCCAACAAGGTAAAATTCACAATGTCTGGCATCC | SEQ ID NO: 275 |
| L1ME_ORF2 | TCGTGACCTTGGGYTAGGCAAWGATTTCTTAGATATGACACMAAAAGCAC | SEQ ID NO: 276 |
| MER89 | AAGCTCTGAATAAATAGCCTTTGCTTGTTCTCATTTGGKTGGTCTTCATT | SEQ ID NO: 277 |
| MER90 | CCTCGCTGCARCGAGCAATAAACCCAACTTGTTCAACCACAGGTGTGTTC | SEQ ID NO: 278 |
| CHARLIE3 | ACAGCAACCAAAACGAGNTTACGGAGTAGACTGGACATAAGCAACACACT | SEQ ID NO: 279 |
| MER91B | ATAATGACAATTTTCCAACAGATGGCAGTAAAGTGTCTTGAGGAAGGGGC | SEQ ID NO: 280 |
| HARLEQUIN | CCTGTACTTCTTCAAATGATAAAAAGCTTCATCGCTACCTTAGTTCACCA | SEQ ID NO: 281 |
| CHESHIRE | TGCCTTCCAAGCAATGAATATGCTCAATTNAAATCATATGCTCGTGATTG | SEQ ID NO: 282 |
| GOLEM_A | GAAATTGCCTAATGACGCATTTCTCAGAACGTATCCCCGTCGTTAAGCGA | SEQ ID NO: 283 |
| GOLEM_B | TCCTGCAAGCTCCATTCATGGTAAGTGCYCTATACAGGTGTACCATTTTT | SEQ ID NO: 284 |
| LTR34 | TGTGTCTGTGGCTCGCGTTTTTCCCGGACATGCCCTAAAGCTGGCTTAAT | SEQ ID NO: 285 |
| LTR35 | CGTGTTAATTTCYATTACATGGRGAGCCCAGGAACCTGTGGTCNNTAACA | SEQ ID NO: 286 |
| LTR36 | CCTGTACTTCTTCCCCCTAAGCTAGCTTTGGAATAAAAAGTCACTTTCTT | SEQ ID NO: 287 |
| MLT2A2 | CAGACTGAAGGCTGCACTGTYGGCTTCCCTACTTTTGAGGTTTTGGGACT | SEQ ID NO: 288 |
| HAL1 | GNAGGGATGGGGACTGCTTTTCGTNATAAGCCTTGTAGNACTATTTGAGT | SEQ ID NO: 289 |
| MER66I | CTGGGCCCCTTAGATCAGGTATCCAGAGATTTTTACTCCTCCGGTGCTAG | SEQ ID NO: 290 |
| LTR37A | TTCCTTCCCCCACTGTGGAAAAAGCCAGTTTTGCNTCYATTTGCAAATTC | SEQ ID NO: 291 |
| LTR37B | GGGAATGTACCTNTGTTGACTTTGCTATTTACTATTTGATTAGGGCCCAG | SEQ ID NO: 292 |
| CHARLIE5 | ACGTTTTCTCACCGATATCACACTGCATATGAACAAGCTAAATTTGAAGC | SEQ ID NO: 293 |
| TIGGER5 | TTAAGGTAGGCTAGGCTAAGCTATGATGTTCGGTAGGTTAGGTGTATTAA | SEQ ID NO: 294 |
| TIGGER5_A | GGTTTCTACTGAATGTGTATCGCTTTCGCACCATCGTAAAGTTGAAAAAT | SEQ ID NO: 295 |
| TIGGER5_B | GTTTACCCTCGTGATCGCGCGGCTGACTGGGARCTGCGGYTCACTGYCGC | SEQ ID NO: 296 |
| LTR38 | ATCTCCCATCTGCTAGCATTTGATTAATAAAGCTGCTTTCCTTTCACCAC | SEQ ID NO: 297 |
| LOOPER | ATGACAGTTGATGAGCAGTTAGTTGCATTCAAAGGATATTGCCCATTTCG | SEQ ID NO: 298 |
| HERVK22I | GCGCCTGACAGACCTGTTGCTGCACACATCTGTACTCTTCAATCAACAAA | SEQ ID NO: 299 |
| MER51I | ACCACCCCTGGTCATTAAGGAGCTACCCTGTCTCCATTAGAHAGAGCAGG | SEQ ID NO: 300 |
| MLT1I | GAGCAGAGCCCCAGCCGACCCGCGATGGACATGTAGCATGAGCAAGAAAT | SEQ ID NO: 301 |
| LTR41 | AGGGGTAGTGGCTGCTCCTTATATCTGCTATTCCTATATTCTTTAGAGTT | SEQ ID NO: 302 |
| MER52A | CAATAAAGCTCCTCTTCGCCTTGCTCACCCTCCACTTGTCCGCGTACCTC | SEQ ID NO: 303 |
| MER52B | TCTCCTCTGAGCTGTTCTATCGGTCAATAAAGCTCCTCTTCATCTTGCTC | SEQ ID NO: 304 |
| MER52C | AGGATGGCCAGAGGACAAAGRGGGCAGAGAGACAATGGGACWGGATGACC | SEQ ID NO: 305 |
| MER94 | GCCTGGGACAGTCCTGGTTTATRCCTGTTGTCCTGGCGTAATTATTAATA | SEQ ID NO: 305 |
| CHARLIE6 | GAGGGGNAACCACACAAAAAGAGNAGGCTAATAAGTTGGCCAAAATAAGC | SEQ ID NO: 307 |
| LTR39 | TTTCTCCCGCTGCAAAATCTCGGTGTSGATGTTTGGTTTTACTGCGCCGG | SEQ ID NO: 308 |
| LTR40A | TCTCTGACCCAGGAGTCTCGTGTCTTCTGCCAGCATCCATGAAACTGTGG | SEQ ID NO: 309 |
| LTR40B | TCTCTGACCCAGGAGTCTCATGTCTTCTGCCAGCATCCATGAAACTGTGG | SEQ ID NO: 310 |
| HERVL_40 | TGCTTGGATGTCCTGTTGATAGTAGCCTTAATTAAATGCTNTATGAGACA | SEQ ID NO: 311 |
| LTR9B | GTGTCGTTTTATCTAAATCGGCGCGAGGACCAAGGACCCTGGTGTTCCTC | SEQ ID NO: 312 |
| HUERS-P3 | CTCCAAATGGTGCTGCAGACCGAACCACACATAGACACGCCATTCTTCCA | SEQ ID NO: 313 |
| HUERS-P3B | GAGATSAAATCAAAATCATTGACAGGCTCAGGGAAAATGCCGGCTTCAGC | SEQ ID NO: 314 |
| HUERS-P2 | TAGACACAGGNAAGAGACCTGGGAAGCTTNAGTAGCCACCGTGTAAGCCC | SEQ ID NO: 315 |
| LTR20B | TTCGCTCCAACCTCACCCTTTGTGTCCATGCTCCTTAATTTTCTTGGTCG | SEQ ID NO: 316 |
| HERVG25 | CTRAGRACCCTTAAACCAGCCTCRRGARAARTCCTAACTGCTGTTNCCTA | SEQ ID NO: 317 |
| LTR42 | CTTCTTTCTTTGGAATCCCAACTGGCCCCATCTCAGGANGGTTTGGGGYA | SEQ ID NO: 318 |
| LTR43 | TTCYTTTGCAATAAATTRQTCTATGCTGCATCTCCTTTGCTGTGTGTCTC | SEQ ID NO: 319 |
| LTR44 | GTGTGTCTTCCCAGGTCAATCCTCACATTTGGCTTCCAATAAACCTTTAT | SEQ ID NO: 320 |
| MER95 | GTCTCCCGGTTCGCGARCTGTWCTTTCTCTYATTGTATGCACAATAAACT | SEQ ID NO: 321 |
| L1MC5 | TAAATGACACCATRGGGATGCAATCAGCAAAATCCAGACTGTGGGAAACT | SEQ ID NO: 322 |
| MLT1J | ATGGAGCAGAGCTGCCATACCAGCCCTGGACTGCCTACGTCTAGACTTCT | SEQ ID NO: 323 |
| HERVFH21 | CAAGACATGATGCTACTCCAAGAATACCGACGGCTCCAGGAACAGCAGTC | SEQ ID NO: 324 |
| ZOMBI_C | AAACTCATTTGGCAGCAAAACCTGACCTGAACTGATATGAGGCTATTTAT | SEQ ID NO: 325 |
| MER96 | AATTTAAGGAGGCACTCACTCTCAGGGTCGTGCAAGTGCAGGGTCGGCAT | SEQ ID NO: 326 |
| LTR45 | GCCCACCTCCTGTCTCCTTGCTGGCCGGTTTTGCAATAAAGCCTTTCTTT | SEQ ID NO: 327 |
| LTR46 | TCTGGCATTAAGCTGGTCCCCCACYTYYRCAGGTTTTNTGCTGGATATAA | SEQ ID NO: 328 |
| MER99 | GCTTTCAACTTGATGTCAGTGGATTCCTTCGAATCAGTAATGTCTCTATG | SEQ ID NO: 329 |
| RICKSHA | AATACGGTTCGTCTGCTCATAACTGTTATACCCGTGCGACTGTCATTAGT | SEQ ID NO: 330 |
| MER96B | CTCAGGCTCCAGTATGAGTNGACACTGCACAGTTRCTGATCCTGTATTTA | SEQ ID NO: 331 |
| MLT1K | TCTTGCCACCACGNGGAGAGAGCCTGCCTGAGAATGAAGCCAACACAGAG | SEQ ID NO: 332 |
| HERVK3I | CCCTTGGACCAGTCTAAAGCACCACATTAACATCTTATATGTAGTCCTTG | SEQ ID NO: 333 |
| LTR22A | CGCTGCATACCTGTGTCTGAGTACTCATTTCATCCATCGGTCGGCCAGGG | SEQ ID NO: 334 |
| LTR47A | ACACAGACGTGGCTTCTGTTTGTAAGTCCCTATTAAATGTTTCTTTCTGA | SEQ ID NO: 335 |
| LTR47B | TCCTTCTGCGTTTGGGGGTCATTTTGCATATACGGCCCTTTCACGAAACA | SEQ ID NO: 336 |
| MER101 | TTCGTTTTACACCGAAGGCTGCATCTCCCCGGTTTGCAAACTGTTCACTG | SEQ ID NO: 337 |
| LTR48 | CAGTTCATTTCAGCAAACCTTGAGAGGGGACAGAGGGGAAGCTTTCCTTT | SEQ ID NO: 338 |
| LTR48B | TAATCATTCTCCTCTGTGATTCCCCCATGCTATGCACGTTAAAATAAATT | SEQ ID NO: 339 |
| LTR49 | TGCCTTTTGTCAGTTGATTTTTCAGCGAACCTTCAGAGGGCGAAGGGGAA | SEQ ID NO: 340 |
| LTR8A | CTCTTTCTTTATTGCAATGCCATGGTCTTTGTCTGTGCAGCGGGCAGGAA | SEQ ID NO: 341 |
| MER41E | GTAGAAGCCCCAAACCCYMTTGGCGCAACTCWCTCTCTTGAGTATGCCCG | SEQ ID NO: 342 |
| MLT2E | TCCCCCCTCCAGACCTTCACTTCCCCAGCTCCTCCCACAATTGTATAAGG | SEQ ID NO: 343 |
| LTR50 | TCTCTGTTAAAATAACTGGTGTGGTTTCTGTCTTCTCCTGACTGGACCCT | SEQ ID NO: 344 |
| LTR51 | TCTTTGAAGAGAGAGCGCCTTTGGTCTATGCCAGAGACTATCTCTTCCCA | SEQ ID NO: 345 |
| MER103 | GTGCATTGTGAATCTCCAAGAGGGGAAATATAGTATGCAGTRTTTCCCAA | SEQ ID NO: 346 |
| MER104 | TTAACATCTCTGAAATCGGGATGCATCTTACAATCGATGGCATGTCATAG | SEQ ID NO: 347 |
| CHESHIRE_A | ACAACGGCAGAGTTGAGTAGTTGCGACAGAGACCGTATGGCCCGCAAAGC | SEQ ID NO: 348 |
| CHESHIRE_B | ACAACGGCAGAGTTGAGTAGTTGCGACAGAGACCGTATGGCCCGCAAAGC | SEQ ID NO: 349 |
| HUERS-P1 | ATCTGCTCTTCGCCTTGCCCAGAGACCCCACTGTGAATTACCATTTGGAG | SEQ ID NO: 350 |
| LTR45B | GTATTGGCTTCGCATCAGGCAGCAGNNAGCCCATTGATTGCTTRGTAACA | SEQ ID NO: 351 |
| LTR52 | ATACCCTCTTGGTGTGTGTGTGGCATCATCAGTCTTAACATCCAAACCAA | SEQ ID NO: 352 |
| MER105 | GCCCTAAGGCATCCATTGTATGTAATGAATTAACTTCTCTCCTATGCATC | SEQ ID NO: 353 |
| LTR53 | CATCTGTCCAGTGTTGGGTGTCATGTGTTTARCCATCCCCATAACCCTAG | SEQ ID NO: 354 |
| LTR54 | TATAAAGCCAACCTCCTCTGCTCAGCTCATYGGAACACTCATTCTATTTT | SEQ ID NO: 355 |
| MER106 | TGTGGTATTAAAATTTCATGGNGGGGGGGGGTGATTAGGAAAAAAATGTC | SEQ ID NO: 356 |
| MER107 | TTCTACTTATCACTAGAGACAGAAACTAAAAACCATGGCTTCAGGCTGCT | SEQ ID NO: 357 |
| MER44B | ACTTAATAATGGCCCCAAAGCGCAAGAGTAGTGATGCTGGCATATTGTTA | SEQ ID NO: 358 |
| MER61I | CTACTGACAGCAGGGGAGATAGGGCATACGTGGGTAGAGCGGATAATTCC | SEQ ID NO: 359 |
| HERVL68 | CCCTGGAAGGCTTTCAGGTCAGCTTCAACTTACTGGCCAGAGTTGTGCTG | SEQ ID NO: 360 |
| MER83B | CCTCTTTGCAGACAGCCCCTTCTCTGCTGTGCTGCCCGTTGCAACCTTGC | SEQ ID NO: 361 |
| MER83C | GCACGTAGCCCCCTCCAGTACAACCCTATAAAACTTCCCTCCAGCCCCTG | SEQ ID NO: 362 |
| MLT1L | GAAAGAACCTGGGTCCTTGATGATATCGTTGAGCCGCTGAATTAACCAAC | SEQ ID NO: 363 |
| MLT2F | ATCAGACGCARAGACAACAGCGTTACAGAGACTGCTTAACCAGCTCCCAC | SEQ ID NO: 364 |
| LTR55 | TCATATCTTTTTCCTTGATCAGCCCCCAAATCCCTTRAACCCCCTTCACA | SEQ ID NO: 365 |
| LTR56 | CTCTTTTTTGCCTTTAAAAATCCACTTGTAACTGCTGCTAATTGGAGTGT | SEQ ID NO: 366 |
| LTR57 | GAGTGCCCTGTATGTAAGTCCTAATAAACTCATCTACTTATCAAGCTGGA | SEQ ID NO: 367 |
| LTR58 | AGCGGCAAGCCTATTAAACCTTGCCTGAGAAAATCGGTTTGGCCTGGTGT | SEQ ID NO: 368 |
| LTR59 | ATTTTTCCTRGRTGTGCCCTCAAGCTGGCTCAGTAAACCTCGATGNTTTG | SEQ ID NO: 369 |
| MER4BI | CTGANAGGATAAAGATACCTCGTGACAAAGCCTCCTGGGTATAATACTCC | SEQ ID NO: 370 |
| MER50I | AAAATGGCTTCCCTGGGTTCTTCCCTTTTTAGGCCCACTTGTTAGTCTCC | SEQ ID NO: 371 |
| LOR1I | TCCAATTACAGGTGTGACGTTTTCATTCCTCATCATTATCCCACAACGCC | SEQ ID NO: 372 |
| LTR26E | TCGGTGTATTGACTTGCCGCGCATCGGGCAACAAACCTATTACGGTCACA | SEQ ID NO: 373 |
| LTR16A1 | CTGCCCTATCCTGCTTCCCTCACTCCCTTACAAGTTTCTCCTGAGAGCAC | SEQ ID NO: 374 |
| LTR24B | TCTTTGGAATCTGTGYTTCCNGGGTGGNCCATCNTCAAACTTTGCACTTG | SEQ ID NO: 375 |
| LTR16D | CCCGCTCCTGCTCCCTCCCCTTTTATCTTTCACAGGNTTTCCCCTAATAA | SEQ ID NO: 376 |
| LTR60 | CTTCAARAAAAATCYGACATCATAAAAACCCCGTGCAGACTCTCAGGGCT | SEQ ID NO: 377 |
| MLT1E1 | GTAGGCAGAATTCTAAGATGGCCCCCAAGATTCCCACCCCCTGGTGTACA | SEQ ID NO: 378 |
| MLT1J1 | TAGCCAACGGAATGTAAGCAGAAGTGATGTGCGCCACTTCCAGGCCTGGC | SEQ ID NO: 379 |
| MLT1J2 | CCTGAGTCACTACNTGGAGGAGAGCCACCCACACCCGACCAGAACCCNCA | SEQ ID NO: 380 |
| LTR1B | TCRGCTRGGGRCRGTCAGAGARGAGNTCAGCCGCTGGAYNGCCAAACTCC | SEQ ID NO: 381 |
| MER109 | TGTCCRTCATTNCTGGCATNGTCAGGACTAGGTAMGGTCTCGDCCAACTG | SEQ ID NO: 382 |
| MLT1E2 | GCCCCCCAAAGATGTCCATGCCCTAATCCCTGGAACCTGTGAATATGTTA | SEQ ID NO: 383 |
| LTR22B | CACTGGCTGGTCGGCAACTGTTTACAGCACTCTCCTGGGAGTCTGTAAGC | SEQ ID NO: 384 |
| MLT1G1 | TTTCCAAAGATGGCCGCAACAATATCTCCCATCCCACATGCTCTTCTTAC | SEQ ID NO: 385 |
| L1MCC_5 | GCCCATTTCCAGGCATAAATACTATTTACCTCAGTCTCTACTGTTCTTCT | SEQ ID NO: 386 |
| MER110 | CTCGCCTCACTGTGCCCACCAATCCAAAGCTATTATGTCATAAACTCTGC | SEQ ID NO: 387 |
| HERVK11I | CAAAGAATCCTGCGTCAAAATCGAGAGAACGAACAAGCCTTCATCGCCAT | SEQ ID NO: 388 |
| HERVK14I | AATAAAAAGGCTGGACAAGATATATGGTGGAGGGATGCACATACAAAGAG | SEQ ID NO: 389 |
| HERVK13I | CAGGCGTCTCCACGGAGTCCAATGAAAAACTCGAAGCCAGCGACAAGCAA | SEQ ID NO: 390 |
| HERVK14CI | CTCATAGCTCCTATAATGCCATTGAACACCAGTGAGAGACGATTAGACGT | SEQ ID NO: 391 |
| LTR14C | ACCGCCACTGCTACACATCTTATCGAATGACTCACGAGTTCTCCTTCACT | SEQ ID NO: 392 |
| LTR61 | ATCCACTGAGCTGGTGCGTACCTTAAAATAAATAACAATCCTCCTGTATT | SEQ ID NO: 393 |
| HERV49I | CTCAATTTGTTTTCTCCCCTCCTTTGCCTATCTCTATCTAACAACCTCTA | SEQ ID NO: 394 |
| HERV15I | ATAGAGGCAGTAGTAACCCGAAACACTACCATGCTATTGACGGCATTAAC | SEQ ID NO: 395 |
| LTR62 | CAAANATGTGTGGACCTGGTTATCTCTGACCTTGCRCTGCTCACGACACA | SEQ ID NO: 396 |
| LTR64 | GGCTATAGGCNTYCCTCAGTCTACAGTCCTCAGTAAGACTTCTGAATAAA | SEQ ID NO: 397 |
| MER112 | CCAGACCAGTGGCTTTCAAACTTTTTTTGACTATGACCCACAGTAAGAAA | SEQ ID NO: 398 |
| MER113 | AAGCACCAAACTGAGACTTTCTCCTTGATGTAATCAGAAGGATTGAAAGA | SEQ ID NO: 399 |
| MER110A | TTACCCAATCCTAATCAAGCCCCTACATTGAAAGACCTGCCTTAAATCAG | SEQ ID NO: 400 |
| LTR33A | CTTCTTGCTGTTGCTAATCTCTGGGTTGCCTCACCATTGNTTCCCTGTTT | SEQ ID NO: 401 |
| MLT1F1 | CCCCGGCCGACATCTTGACTGCAACCTCATGAGAGACCCTGAGCCAGAAC | SEQ ID NO: 402 |
| SATR1 | ACACCCCCCCCSTACVCCCACMCCCCCTGTGATATTGTTCGTAATATCCA | SEQ ID NO: 403 |
| MER115 | TTTAAATA1TTAGACATATGGTATGTGGGCCTCCATTTGTACTCTTGCCC | SEQ ID NO: 404 |
| MER117 | GCACAGGAGGGGGAAGTAGCAGCANATATGCTATGTATTTGCCATCCCTG | SEQ ID NO: 405 |
| MER20B | TAGGTGCAAGCATCTGACTACTTCATTATGTCTTCTAGTGTAGTCATGCC | SEQ ID NO: 406 |
| LTR65 | TCCATGGTTCCTCTGGTGTGCAGTCTCCCTCATTGCAATAAGTCAATAAA | SEQ ID NO: 407 |
| LTR38B | TGAAGYGGTTGCTTTGGATAGGAATCYGGCCRCTTCCCCATTACTAGTTT | SEQ ID NO: 408 |
| CR1_HS | GGATTGACAGCAGATCAMGGGAAGTGATTATACCCCTTTACAATGCCTTG | SEQ ID NO: 409 |
| L1ME4 | GTGGGATGGACAGGGATGGGAGGGACTGACTTTTCACTGTATACCTTTTT | SEQ ID NO: 410 |
| MLT1H1 | TGGACCCTCCAGACCAGCCCATCTGCCAGCTGAATACCACTGAGTGACCT | SEQ ID NO: 411 |
| LTR2B | GGGACAGAAATTGTGCACTCGGGGAGCTCGGATTTTAAGGCAGTAGCTTG | SEQ ID NO: 412 |
| MER101B | CCAGAAACCACCTCCCCACAAGCCCACTAGAAACAAACATCTGACAGAGA | SEQ ID NO: 413 |
| MER45R | TAGCGNATAAAATACTCTTAACAGCTOCAGNAACAGTTGCATCAGCAGAA | SEQ ID NO: 414 |
| MLT1G2 | TTTAAAACATGGCCGCAAATTCTTTGACACTCCTCTCATTGAGANGTGGG | SEQ ID NO: 415 |
| MSTA1 | CTTGCTTCCTOTCTCACCATGTGATCTCTGCACACGCTGGCTCCCCTTCC | SEQ ID NO: 416 |
| LTR6A | GAATTCGTCTCAAAGTGTGGCGTTTCTCTATAACTCGCTCGGTTACAACA | SEQ ID NO: 417 |
| L3 | GGTCTGGAAACCATGTCATATGAGGAACGGTTGAAGGAACTGGGGATGTT | SEQ ID NO: 418 |
| LTR66 | TGCCATTTACGTGGGATAAAGCTTGTTTACCCTTAAAGGTATTGTGTGTG | SEQ ID NO: 419 |
| PRIMA41 | ACCTTTTGTCGGAACTCGGAGTTATGAACGACGCTCACCATACCGATGCT | SEQ ID NO: 420 |
| MARNA | TATNGCCTCCCAAGGTGACTACTTTGAAGGGGACAACACTCATTTGGATG | SEQ ID NO: 421 |
| MER119 | TTACTGAGACACTAAGGGCGCCGTGAACCGAGAAAGTTTGGGAACCTCTG | SEQ ID NO: 422 |
| LTR67 | GTTCTCCAGCCCTCCCGGAGATTCTGTGAGCTACCCAATATCCTTTAATA | SEQ ID NO: 423 |
| L1M3DE_5 | CGGGCNGATTGGTGAGATCCNTCTCCTACACGAGGCCAGTCTGACAAGAC | SEQ ID NO: 424 |
| RICKSHA_0 | CTCTTATGGACTATCTCCGTGGAATTGCCCATAATCTATCCCTGTAATAT | SEQ ID NO: 425 |
| MER4E | AGGGGTCTGGGGAGTCATGCCGTACAAACCATAAATTCTCATCAGATGGG | SEQ ID NO: 426 |
| MER104A | ACCTTTCGCGTTTCAGTTAACAAACCATTTAAGGACCATTTGAGGAAGGA | SEQ ID NO: 427 |
| LTR40C | TGCTCATGCTGCTTGCTGTGYCATGAGTAATAAAGTCCTTTGTCTCTGAC | SEQ ID NO: 428 |
| LTR54B | TGCTCAAGCTACTTTAQAAAAGCCAAACTGCTCTGCCATGCCCAGCGGAG | SEQ ID NO: 429 |
| MIR3 | GGAAGCAGTATGGTATAGTGGAAAGAACAACTGGACTAGGAGTCAGGAGA | SEQ ID NO: 430 |
| MLT1G3 | CCAGCTGTCAAGTCATCCCCAGCCTCTNNCAGYCMTCCCCAGCCTTCAAG | SEQ ID NO: 431 |
| MSTA2 | CCACTTCCCCTTTGACCTTCTCTGCCATGTTATGATGCAGCATGAAAGCG | SEQ ID NO: 432 |
| L1MD1_5 | TTTGAGAACTGAACTAAAGGATAGACCACTACCCAGGTCCCAGACTGGCC | SEQ ID NO: 433 |
| LTR10E | ARTGCTAATTTTTCTTTGCAGCACCGAGGAACAAGCATTCTGTTTCTAAA | SEQ ID NO: 434 |
| LTR24C | TCTCTGGAGTCTGTGTTTCCTGAATGGCCATTCCCAGCTTTTNACTTGAA | SEQ ID NO: 435 |
| MLT1C1 | TGGAGTGATGCAGCCATAAGCCAAGGAATGCCAGCAGCCAAGCCACCAGA | SEQ ID NO: 436 |
| MSTD | GTGGGTTTGTTATAAAAGNAAGTTCGGCCCCCTTTTGCTCTCTCNCTCTC | SEQ ID NO: 437 |
| LTR68 | ATCTTTACGTCATATACATTTCCATGTCTCAGGAGGCTAGGGCTTTTTAC | SEQ ID NO: 438 |
| L1MED_5 | TAAAAACCCAGTGGATAGGTNAAACAGCAGATTAGANACAGCTGAAGAGA | SEQ ID NO: 439 |
| L1ME5 | ACTGAAAGGAAATATACACCAAAATGTTAACAGTGGTTATCTCTGGGTGG | SEQ ID NO: 440 |
| TIGGER6A | TAGAAGAAATAGCTGACCGTGGGAATGTTGACACTGCCGCCATTTGAGAG | SEQ ID NO: 441 |
| MER51C | AGACCAAATCCTTCATCCAGATAAGGGGTAGCCAATAGGAACCTCAAAAG | SEQ ID NO: 442 |
| LTR6B | CCGGCTAAATAAACGGACTCTTAATTCGTCTCAAAGTGTGGCGTTTTCTC | SEQ ID NO: 443 |
| MER21A | TCCACAGTTCCTGGCTCATAACTCCGATAGCCCTTGTTACAGTCTTTTGT | SEQ ID NO: 444 |
| MER34B | CCACAAGTTGCTGCCCCTAGAGACTCAAAGTCCTTTTCCTTTGTCTTGTC | SEQ ID NO: 445 |
| LTR3B | AGTTTCTTTTGTCTTAAGTTTTCATTTCTGCGTTCGTCCCCCTTCGTTCA | SEQ ID NO: 446 |
| MER54A | AGGCGGTTGTATAAGGCAGATATCTGGATCGACCACATTGAGGAACTGGG | SEQ ID NO: 447 |
| MER74C | GCCTTTCATCTATCCGAGTGTCANTGTGTTGTGTCCCGCCATCAAAAGAA | SEQ ID NO: 448 |
| ERVL | AAGAGTAAACATCACTCAAGGACTTTACCTCCTCTTCTGGGGAAGGGGTT | SEQ ID NO: 449 |
| HERVL74 | AAATACCCCNAATAATTGATGTCAAAACTGACGTCAAGACANAAAGGGGT | SEQ ID NO: 450 |
| MER83AI | TAAGTCCCAACTCAGGGATTTAGGTCCACGTAACCTCCTGACCGACTAAC | SEQ ID NO: 451 |
| MER83BI | TCTCCGATGAGTTCTTTCCTCCAGCAAGATCCAATATCCTAAGTCCCACA | SEQ ID NO: 452 |
| MER84I | ATTTTCCCTTTCTTGAGACCCCAATAGGCAGCAGGTAGACATGAGCATGG | SEQ ID NO: 453 |
| LTR75 | TAATAAACTGTCTGAATCTAAAAGTGGCTCGTTGTATCTTTACCAGCCGA | SEQ ID NO: 454 |
| L1PA7_5 | CACCGAGCTAGCTGCAGGAGTTTTTTTTTTTCGTACCCCAGTGGCGCCTG | SEQ ID NO: 455 |
| L1PA13_5 | CTTTAGCCCTAGGGGAACTGTCGGACCTGAACTCTGCAGGGCGGTCTTGC | SEQ ID NO: 456 |
| L1M1_5 | AAGAAACAAATAACATACAATGGAGCTCCAATACGTCTGGCAGCAGACTT | SEQ ID NO: 457 |
| LIM2A_5 | CATGTCAGACCCGACACCAAGAGGGATCCCCTCGGCTAAGTCTCCCCATT | SEQ ID NO: 458 |
| L1M1B_5 | CCCATTCGGGACGGGCAGCGCTCTGATTGTTTACTAGAGCCGAGGCAAAC | SEQ ID NO: 459 |
| LIMB3_5 | AAAGGGGTGGGGATGGAGCTGTAAAGGAGCAGAGTTTTTGTATGTTATTG | SEQ ID NO: 460 |
| L1MDB_5 | CACAAAAGTAGGCCAGGACCTGCATGCTAAACCTAAACAGGGTGACTGCC | SEQ ID NO: 461 |
| L1HS | CACAGGAAGGGGAATATCACACTCTGGGGACTGTGGTGGGGTCGGGGGAG | SEQ ID NO: 462 |
| L1PA3 | AACACATGGACACAGGAAGGGGAACATCACACTCTGGGGACTGTTGTGGG | SEQ ID NO: 463 |
| L1PA4 | AACACATGGACACAGGAAGGGGAACATCACACACCGGGGCCTGTTGTGGG | SEQ ID NO: 464 |
| L1PA5 | GAACACTTGGACACAGGAAGGGGAACATCACACACCGGGGCCTGTTGTGG | SEQ ID NO: 465 |
| L1PA6 | GAGAAATACCTAATGTAAATGACGAGTTGATGGGTGCAGCAAACCAACAT | SEQ ID NO: 466 |
| L1PA8 | AGGACAAATACCTAATGCATGCGGGGCTTAAAACCTAGATGACGGGTTGA | SEQ ID NO: 467 |
| L1PA10 | ATAGCTAATGCATGCTGGGCTTAATACCTAGGTGATGGGTTGATAGGTGC | SEQ ID NO: 468 |
| L1PA12 | CTTAATACCTGGGTGATGAAATAATCTGTACAACAAACCCCCATGACACA | SEQ ID NO: 469 |
| L1PA13 | TACCTGGGTGATGAAATAATCTGTACAACAAACCCCCATGACACAAGTTT | SEQ ID NO: 470 |
| L1PA14 | GGGAGAGGAGCAGAAAAGATAACTATTGGGTACTGGGCTTAATACCTGGG | SEQ ID NO: 471 |
| L1PA16 | TGGGTGATGGGATCATTCGTACCCCAAACCTCAGCATCACGCAATATACC | SEQ ID NO: 472 |
| L1PB2 | ATCTCAGAAATCACCACTAAAGAACTTATCCATGTAACCAAAAACCACCT | SEQ ID NO: 473 |
| L1PB4 | KTACACTAAAAGCCCAGACTTCACCACTACGCAATATATCCATGTAACAA | SEQ ID NO: 474 |
| L1MA1 | ATTCTCCATGATGTGCTTATTTCACATTGCATGCCTGTATCAAAACATCT | SEQ ID NO: 475 |
| L1MA3 | GCTGGGAAGGGTAGTGGGGTGGGGGGGAAGTGGGGATGGTTAATGGGTAC | SEQ ID NO: 476 |
| L1MA4 | GGAGGGGGGGAATGAAGAGAGGTTGGTTAATGGGTACAAAAATACAGTTA | SEQ ID NO: 477 |
| L1MA4A | GAGGACTTGAAATGTTCCCAACACATAGAAATGATAAATACTCGAGGTGA | SEQ ID NO: 478 |
| L1MA5A | TGGGAAGGGTAGGGGGAAGGGGGGGATAGGGAGAGATTTGTTAAAGGATA | SEQ ID NO: 479 |
| L1MA6 | ATAGGAGGAATAAGTTCTGGTGTTCTATTGCACAGTAGGGTGACTATAGT | SEQ ID NO: 480 |
| L1MA7 | ATGGGGAGATGTTGGTCAAAGGGTACAAAGTTTCAGTTAGACAGGAGGAA | SEQ ID NO: 481 |
| L1MA8 | TGCTNATGGTCCCATGACTGGCCACTCTGTGAACACAGTAAACAAGTTTG | SEQ ID NO: 482 |
| L1MB1 | GAAATGGGGAGTTGCTGTTCAATGGGTATAAAGTTTCAGTTATGCAAGAT | SEQ ID NO: 482 |
| L1MB2 | GGGTATAGAGTTTCAGTTTTGCAAGATGAAAAAGTTCTGGAGATCGGTTG | SEQ ID NO: 484 |
| L1MB4 | TGGTGATGGTTGCACAACAMTGTGAATGTACTTAATGCCACTGAATTGTA | SEQ ID NO: 485 |
| L1MB5 | AGGGGGAATGGGGAGTGACTGCTTAATGGGTACGGGGTTTCCTTTTGGGG | SEQ ID NO: 486 |
| L1MB8 | GGAATGGGGAGTGACTGCTAATGGGTACGGGGTTTCTTTTGGGGGTGATG | SEQ ID NO: 487 |
| L1ME1 | GGTGGGGGNAGGGGATTGACTACAAAGGGGCATGAGGGAACTTTTTGGGG | SEQ ID NO: 488 |
| L1ME3 | ATAGTGGTTACCTTTGGGGAGGGTTATTGACTGGGAAGGGGCATGAGGGA | SEQ ID NO: 489 |
| L1ME4A | GACTGGAAGGAAATACACCAAAATGTTAACAGTGGTTATCTCTGGGTGGT | SEQ ID NO: 490 |
| L1MC1 | TTGATAGTGGGGGAGGCTGTGCATGTGTGGGGGCAGGGGGTATATGGGAA | SEQ ID NO: 491 |
| L1MD3 | ACCCATAACCCCAGTCTAATCATGAGAAAACATCAGACAAACCCAAATTG | SEQ ID NO: 492 |
| HAL1B | AGAGGAGAGGTGGAAGGAAGTATGAGAGTGCTAATNTCCTCATCTTTCAT | SEQ ID NO: 493 |
| L1MA9_5 | AGACCCAGGGTTCAGGCCTGTCCCAGTAGACCCCAGCACTAGGCTAGTCC | SEQ ID NO: 494 |
| L1MDA_5 | AAGAAGGAATCTTGGAACATCAGGAAGGAAGAAAGAACATAGTAAGAAGC | SEQ ID NO: 495 |
| L1MEB_5 | GGCAGAAACTGGAGGGGAGTCGACACCTGGAAGAAGGGAATWGCACGGAG | SEQ ID NO: 496 |
| TIGGER5A | TTAAGGTAGGCTAGGCTAAGCTATGATGTTCGGTAGGTTAGGTGTATTAA | SEQ ID NO: 497 |
| T1GGER6B | AGGCAACCCCATCAAGAACTTANGCGAAAAAAGATGTAGGATCACAAAGT | SEQ ID NO: 498 |
| TIGGER7 | TCGGATGGAACGCAGCATTAAAGTCACCCATATGATCAATGAAGGATTAC | SEQ ID NO: 499 |
| MER44D | CCTCACTTCATCTCATCACGTAGGCATTTTATCATCTCACATCATCACAA | SEQ ID NO: 500 |
| MER69C | ATCGACGAAGATAACATAAAACTCATAATACGCCACTACAACGAGGACAT | SEQ ID NO: 501 |
| MER106B | TATTTATGTTTGATCGTCAGTGCTTTGTGTGACTTGGGCTTTGAGAATTA | SEQ ID NO: 502 |
| CHARLIE2A | GATTGGTTTGACAATGAGGACTGGCTTTGCCAATTAGGTTATATGGCAGA | SEQ ID NO: 503 |
| CHARLIE2B | TTPATNCACCTTTTGTAAGCCCTATACTTACTAGTGGCCCAATACCTTCT | SEQ ID NO: 504 |
| CHARLIE7 | ACTTAGAACCAGACCTTCGAATCGCTGTATCACAAAGTGTTAAACCAAGA | SEQ ID NO: 505 |
| CHARLIE8 | ATTTATGTTACCTGCCTGGCCCCTGTAGGCATTTGAGTTTGCGACCCCTG | SEQ ID NO: 506 |
| CHARLIE8A | ATTTATGTTACCTGCCTGGCCCCTGTAGGCATTTGAGTTTGCGACCCCTG | SEQ ID NO: 507 |
| MER63D | ACAATGTAACGGCTACAGACACGACACACTTTTAAGTTTAATCTGCATTA | SEQ ID NO: 508 |
| MER97A | TGTTAAAAAATGATCCGCTCTGGGTGTCGAATACGCTAGGTACGCCACTG | SEQ ID NO: 509 |
| MER97B | CCAGTGGTATGNTTTWGTAGTTGCCTAAATTGTACCTTTTGCAGACGTTT | SEQ ID NO: 510 |
| MER97C | TGTTAAAAAATGATCCGCTCTGGGTGTCGAATACGCTAGGTACGCCACTG | SEQ ID NO: 511 |
| MER6B | GTTCTTGGAAACTGCGACTTTAAGCGAAACGACGTACAGCAGGTCCTCGA | SEQ ID NO: 512 |
| ZAPHOD | ATTGCCGGCCCATCAACAGAACACCCAGACATGTGCAATAATAATTAAAT | SEQ ID NO: 513 |
| TIGGER9 | GCCAGTCAGATTTCACGGCANTGCCAATGTTTCTGTCTGTACAGCGNTGT | SEQ ID NO: 514 |
| HERVL66I | CTCCTGTGCTTACCCTGTATCTGTAATCTATATCAACTATGCCTTCCCCA | SEQ ID NO: 515 |
| THE1A | TTTATCAGGGGTTTCCGCTTTTGCTTCTTCCTCATTTTCCTCTTGCCGCC | SEQ ID NO: 516 |
| THE1C | GTGTCCCCACCCAAATCTCATCTTGAATTGTAGTTCCCATAATCCCCACG | SEQ ID NO: 517 |
| MSTB | TGTTAGTTCACGCGAGATCTGGTTGTTTAAAAGAGTNTGGCACCTCCCCC | SEQ ID NO: 518 |
| MSTB1 | CTTCCTCTCTCGCCATGTGATCTCTGCACACGCCGGCTCCCCTTCACCTT | SEQ ID NO: 519 |
| MLT1AR | TCAGTCTGCTCCCTATCTTCGGCTGCCCGTTTAGNTGTGGCTCAAGTGGG | SEQ ID NO: 520 |
| MLT1CR | AAGGTGCGGCCTGGTTTCTCCTTGCTGCTTATAGTAAAATGCGAGAGGAA | SEQ ID NO: 521 |
| MER104B | CCTTTCGCGTTTCAGTTAACAAACCATTTAAGGACCATTTGAGGAAGGAA | SEQ ID NO: 522 |
| MER104C | TGAAGGCAGGAGAAATTGCCNAATCCCNCGGAATAGATGAAAGAAATTTC | SEQ ID NO: 523 |
| HSTC2 | TNATGTAGACTCCTTCGCAAGACTCCATCAGCGAACCATTTGACACTTTT | SEQ ID NO: 524 |
| L2A | ACGCTCTTCCCCCAGATATCCACGTGGCTSGCTCCYTCACCTCMTTCAGG | SEQ ID NO: 525 |
| L2B | CCTGCCACTCTGGGTTATAAATTGTCTGTKNGCANGTCTGTCTCCCCCACT | SEQ ID NO: 526 |
| MER51D | TTTGTTTGGGACACCAAGAGCCTGGAACTGCACRGCACCAKCTGGTAACA | SEQ ID NO: 527 |
| MER5C | TGGACCAGTGCTAGTCTGCAAACTGTTTGTTACCAGTCCATGATAAGATA | SEQ ID NO: 528 |
| HERVK11DI | CCCGGTGCTGAAGTTTTAGACGGTATCTCTGAGGGGTTATCTAATCTCAA | SEQ ID NO: 529 |
| LTR69 | GAAAAGTCGCCCCTGGGGAAGCTGGTTAACTAGGACCACCCAAGACCCCC | SEQ ID NO: 530 |
| HERV30I | AAAAAAGGAGCTTGAACACTCAGAACCCTGAAATATGTTTAACCAATGGA | SEQ ID NO: 531 |
| HERV19I | CATAGCAGGAATAATGGTTACTAACAGAAAATAACACATGGGCCTTTCCA | SEQ ID NO: 532 |
| LTR19C | TCACTCTGTGTGTGTGTGTCCGCGACCTCGATCTCCTTGGCCGTGAGACC | SEQ ID NO: 533 |
| HERV46I | ACCCACTGCTTCAAAACCCAAACCCTGATTACAGCNCCCCTATTCGGCAG | SEQ ID NO: 534 |
| HERV52I | TNAATAAGACATGGCACATTTCAGTCATCCATCAAACATCAGGGGTGAAT | SEQ ID NO: 535 |
| MER89I | GCTTCTGCGCAGCCGCTCTCTCATCAGATGATCGCCATGATGATACAACA | SEQ ID NO: 536 |
| MER110I | GACAATGGTCTNTCCTTCAGNTCGGGNTGAAGAATGACCAAAGGAGAAAT | SEQ ID NO: 537 |
| MER21I | ATCCTTGTTTCGNTGTAAGGGATTCAGTGGTTGGAAANCAGGGAGTGGCC | SEQ ID NO: 538 |
| PABL_AI | GCGCTCAAAGGGTGAGTTAACTGGATCGTATGCCGGGAGCCTATTGTTTT | SEQ ID NO: 539 |
| PABL_BI | CTCGCGGTCCTGGCCATCCTTGNAGGCATGGGCATAACGTTATGTTGTGG | SEQ ID NO: 540 |
| MER52AI | ACNCCCANGGGATTATCTACTCCCCTAAACAGCTATCTCTCTTCTAAAGT | SEQ ID NO: 541 |
| HERV57I | AGCCATGGCTATACGTTATAGACCTGTATAGTTCTTCCCCTCATACCCTA | SEQ ID NO: 542 |
| MER70I | GGGCATATGAAATGGACTAGCTTTGCTAAGGGGGATATCTGGGTTGGGGG | SEQ ID NO: 543 |
| HERV38I | CGGGATCGGTTTGGAGTGCTCCGTCTGCATCGGATCCGTCTGTGTTTGTG | SEQ ID NO: 544 |
| L1M2B_5 | CTTTCCCTACCCACTGCCACTACNYCTGACTCTGGGGCCAAAGCACATGC | SEQ ID NO: 545 |
| L1M2C_5 | ACACCCCAATGAACTGACACCAAGACCCATTTATACAAATAAGTTTTTCC | SEQ ID NO: 546 |
| HERVFH19I | CTGGAGCAGTCCTCCAAAATAGACGGGGATTAGATCTTATAACGGCTGAA | SEQ ID NO: 547 |
| HERV70_I | CTCAGTGGCAGATGGTAGAGGTCAAGAGAGGANGGACACTAGCAACCAGG | SEQ ID NO: 548 |
| LTR70 | TCTTTGCTCCCAGGTTAYAATCCTNAAGCTTGRCCCAAATAAACTGTCTA | SEQ ID NO: 549 |
| MER120 | AGATGTGGATACTCAAGATTTCTATTGGGGAAAACTGTGGTCCTTAGTAA | SEQ ID NO: 550 |
| REP522 | TGTATTGCTGGCAGCAGTGAGGTGGGTTAAGGGTGCTATCCGGGGCTGCA | SEQ ID NO: 551 |
| LTR71A | TTAAAAGTCTCGCTTCCACTGTTCTTCGTGTCTCTGAGTCCATTCTTTGG | SEQ ID NO: 552 |
| LTR71B | CATTAAAAGTCTCACTTTCGCTGTTCTCCGGGTCTCTGAGTCCATTCTTT | SEQ ID NO: 553 |
| LTR12B | CCCACCAGAAGGAAGAAACTCCGGACACATCTGAACATCTGAAGGAACAA | SEQ ID NO: 554 |
| MER121 | AGCACTTTTTTCCCCCCTTAATTTTTAAACCCATGTGTATTTCAAGGGAA | SEQ ID NO: 555 |
| MER122 | TGCAGTTGGTGGCGACAGAGACTGTAGTGTGGCTGGAGTGGTAGGAAGGG | SEQ ID NO: 556 |
| LTR7A | AAAGCTTTATTGCTCACACAAAGCCTGTTTGGTGGTCTCTTCACACGGAC | SEQ ID NO: 557 |
| LTR7B | ACAGCCTTGTTGCTCACACAAAGCCTGTTTGGTGGTCTCTTCACACGGAC | SEQ ID NO: 558 |
| MER51E | GATTAGGCAGCAYACAGGCCACATCCTCACTCCTGTGATAACAAGACAGA | SEQ ID NO: 559 |
| MER4IF | CAGGAGAATAGAAAATTCCAGGCAGCAGTTTCACATGACTAGCAAAAGGA | SEQ ID NO: 560 |
| LTR2C | AAGATAAATAGCCAGACAACCTTGGCACCACCACCYGGCCCTAGGAGTTA | SEQ ID NO: 561 |
| LTR38C | ACACCTCACTCTTGTTATTTTGGCTTCTTTCTACAAGCGGCAAGCAGCYG | SEQ ID NO: 562 |
| LTR72 | AACCTGTATTCTCATGGAGAGTCGTTTGTTACTCACCAGGYGAATRAACC | SEQ ID NO: 563 |
| MER65D | TAAAAGCTTCCCTTTACCCTCCCCTCTTCAGATGCATCTGTGGCTTGCCA | SEQ ID NO: 564 |
| ALR1 | TGAGGCCTTCGTTGGAAACGGGATTTCTTCATATAATGCTAGACAGAAGA | SEQ ID NO: 565 |
| LTR1C | GGTTCCAGCATTCATTCGCTCCGGTTCCCGCACTCACTCGCTTGCATGCT | SEQ ID NO: 566 |
| LTR45C | TCTCACAAGCAGAGGGAGTTTCAGCATTTCAGCAAGTTGTTTCTTTTCTT | SEQ ID NO: 567 |
| LTR76 | GATGTTAAGTCTGCTGGGTCTGAGTGCACTCAATAAAAGATCCTCCTGTT | SEQ ID NO: 568 |
| MER72B | TTTCACAATGCATCCCTTCCTAAAAACTGACCACCATCTCTGGACTGGTT | SEQ ID NO: 569 |
| ALR2 | GTGAAGGGATATTTGGGAGCTCATTGAGGCCTATGGTGAAAAAGAAAATA | SEQ ID NO: 570 |
| LTR1D | GTTCCAGCACTCATGCACTCCAGTTCCCAC0TCGTTCACTCACATGCTCC | SEQ ID NO: 571 |
| MER34C | TCCTGGTCACCTCCCCATAACTGGCCTTCCCCACACCCTTCTTTCTTTGT | SEQ ID NO: 572 |
| MER50B | ACTCCCTAAACACACTGCGCGTGCTCAATTCCCAAGGGTAAGGAGGGCAC | SEQ ID NO: 573 |
| HERVP71A_I | AATTGTGGCAGGAGTCTTAACAGCAGTGGGATGTTGTATTATCCCTTGTG | SEQ ID NO: 574 |
| LTR27B | TTTGCCCACCCTTTCCCGATTGATTCTTTCTGAATAATGCCTTTTAACCA | SEQ ID NO: 575 |
| LTR12C | CACCAGAAGGAAGAAACTCCGAACACATCCGAACATCAGAAGGAACAAAC | SEQ ID NO: 576 |
| LTR43B | CAGTCGGTGCTGTCTCACYYTTGAGCAGCCNYGCTCTGACTCAGCTGTCA | SEQ ID NO: 577 |
| LTR72B | CCCTTGTTAAATCGTCCTTGGTTGTGGTCATTGGACTGTCACCTGCCAAG | SEQ ID NO: 578 |
| LTR77 | GGGACAAGAACTCAGACCTTGCTAAACTAAGGAGTAAGAAGACTGCAACA | SEQ ID NO: 579 |
| L1PREC1 | GTCAAAGTGCTTCATTAAATGGGTCCTGTTCCCTGTGCCACCCAACTGGG | SEQ ID NO: 580 |
| MER2B | TCATTCACGTGGATTCAATGTAGTACTYGGTGTATGGCAAATTCAAGTTT | SEQ ID NO: 581 |
| MER93B | CTATAAAAGCCTCCCCCTTGCATTCCCTCGGTGGAGCTCCCGAACCACTT | SEQ ID NO: 582 |
| SATR2 | TGTACACCCTGTGATATTATTCGTAATATCCTAGGGGGATGTTACTCCTA | SEQ ID NO: 583 |
| GOLEM_C | GGGNAAATGANTGATATTCAGTAATGGTGCTGGGACATTTGGTTTTCCAT | SEQ ID NO: 584 |
| MLT1A1 | CCCCTCTAGAGGATGCAGCATWCAAGGYGCCATCTTGGAAGCAGAGASCA | SEQ ID NO: 585 |
| L1PREC2 | TGGCTGAACACTCCCAGTAACAGTGGCTCTGCGTTTCTCGGAGGTGGAGC | SEQ ID NO: 586 |
| BLACKJACK | CATCCAAACAAGCTGCGATATTCTACCCAACGATATAGAAGCTGTAGTTG | SEQ ID NO: 587 |
| L1M2A1_5 | GCCCACCCAACCCATCACAGCTTCCAGCAACACCAACATGGACTGCTTGG | SEQ ID NO: 588 |
| MLT1E1A | TGGAAGAGGATTCTAAGCCTCAGATGAGAACACAGCCCTAGCCAACACCT | SEQ ID NO: 589 |
| MER4E1 | TTCTTCCAGACCCTCCCAATCCTAAAGAGATTAACTAAGATCTGAATAGG | SEQ ID NO: 590 |
| PRIMA4_I | CGTGACCTCCTAGGAATGAGCCTTCCTAGTGATGTGGGACCTAAACTTCT | SEQ ID NO: 591 |
| PRIMA4_LTR | TTTAAATTTGGAGCCCTCAAAATCATCTTCGGAGAAAGGCATAGACCTGT | SEQ ID NO: 592 |
| L1M4B | AAAACAANCACNANGAGCCGGGGGNGGGGAATCAGTATCCAGAGTTGCTA | SEQ ID NO: 593 |
| L1PA14_5 | CACACAGACAGCAGATTAGGGCTAACCTGGCAAGGATACAGCTTGTCTGC | SEQ ID NO: 594 |
| LTR13A | TCTCTTTGTCTTGTGTCTTTATTTATTACAATCTCTCGTCTCCGCACACG | SEQ ID NO: 595 |
| HAL1C | AACCACAACATNAGAGGACCCANCACTCCTCCTACCACCAAAACAAAACC | SEQ ID NO: 596 |
| HERVIP10F | AGAGGCTCATAGAAATGGCACTTACTAAAACCTCCCTTAACTATCCTCCA | SEQ ID NO: 597 |
| MLT1F2 | CNGATCCTCCCCTCNAGTTGAGCCTTGAGATGAGACTGCAGTCCTGGCTG | SEQ ID NO: 598 |
| MLT1FR | TTTGGACCCCCAAAATTCTACTGGCAGGAAGCAGGCTGAGAAAACTACTC | SEQ ID NO: 599 |
| HERVIP10FH | CAGAGGCTCATAAAAACGGCACTTACTAAAACCTCCCTTAACTATCCTCC | SEQ ID NO: 600 |
| LTR10F | TTCCCTCCCTTGTCCAGGTGTGCGCTCACCATTGCTCCATCTGTGAGGGT | SEQ ID NO: 601 |
| MER34B_I | CTAAAGACACTTTGTGCTCAGACCTAGAAATCTTCTCAATTGGCTGCCAT | SEQ ID NO: 602 |
| MER57A_I | CTGGAAGGCCTATGCACCTAATAATAGAACCTCATGTATCTTCCGCTACT | SEQ ID NO: 603 |
| PRIMAX_I | AATTAACCAAGGCTTTTAAAATTCCTTGGCCAAAAGCTCTTCCATTGGTT | SEQ ID NO: 604 |
| MER75B | CATTTCCCGTTTGCCCCAAGAATACTCTTGTCTCTAATCCTAATGTAACA | SEQ ID NO: 605 |
| MLT2B3 | CCCAGGTGGTTTGGCATTTGATTAGAATGATTGGGCTGCCCCAGGTGTGT | SEQ ID NO: 606 |
| MER66C | AGGATCTGGTCCAGACAGGATAAAGTGAAGAAACNRGCAGGAACCAGCAG | SEQ ID NO: 607 |
| MER52D | CACNGCTCCACACCTGRCTTNNCCTTGGCAGGNNTGGATCNAGGNCCTTG | SEQ ID NO: 608 |
| MER41G | TGCTTTGCAATAAAAGCTTCTTGCCTTTCGCTTCATTCTGACTCATCCCT | SEQ ID NO: 609 |
| MER21C | AGGAGCATCTTTTGTTCTAATATTTGGTCTTTGACCCTAGTTCCTGACAC | SEQ ID NO: 610 |
| LTR20C | CCAACCTCACCCTTTGTGTCCATGCTCCTTAATTTTCTTGGTTGTGAGAC | SEQ ID NO: 611 |
| L1PBA1_5 | TCTGTTTGCGGGAGAAGTTTCTGACTTTACCTGGAGCTGAGTCAAKTTAG | SEQ ID NO: 612 |
| L1MB4_5 | AATCTCATGTCAAAAAAACACTAGCTGAACACAAGCTAAGGAACAGAGAC | SEQ ID NO: 613 |
| LTR73 | TTGACACTCACTTTCGGTTTTGTGTATTGGCTTCGTGACACCAAACAGGG | SEQ ID NO: 614 |
| HARLEQUINL | GGGAGGAGACCACCCCTCATATTGTCTTATGCCCAATTTCTGCCTCCAAA | SEQ ID NO: 615 |
| TR | | |
| LTR12D | CACCAGAAGGAAGAAACTCCGGACACATCTGAACATCTGAAGGAACAAAC | SEQ ID NO: 616 |
| LTR12E | CACTCCTGAAGTCAGCGAGACCACGAACCCACCGGGAGGAACAAACAACT | SEQ ID NO: 617 |
| MLT2B4 | GTAAGAGAGAATTCCTCCTGCCTGACTGCCTTTGAACTGGGACATCGGTC | SEQ ID NO: 618 |
| MER9B | TAACAACATGTTTTTGCTGCAGATAATCAGCCAGAGCCTGTTTCTCTRCT | SEQ ID NO: 619 |
| SVA2 | GAAGTGACAGCCTTGTGTGTGATCTTTCTGCCCTCCCCAAGTTTGCATTT | SEQ ID NO: 620 |
| HERV39 | TCTTGCTGCTAAAACTGCATACAACAGCCACCCAGCCAAGAGGAATTAAT | SEQ ID NO: 621 |
| MLT1H2 | CCCAGCTGCCATGCTAAAAGAAGCTCAGGCTAGACTATTGGATGATGAGA | SEQ ID NO: 622 |
| LTR10G | GCTGAGAAAACTTTTGCCTGAGTGCTGGTTTCACTTTGCGGCACCAAGCA | SEQ ID NO: 623 |
| MER4A1 | CAGAAACTCAAAAGAATGCAACCATTTGTCTCTCACCTACCTGTGACCTG | SEQ ID NO: 624 |
| MER4D1 | CTCTAGTATAGCATCACATGAOAGATAGCAGGCCCTGAAAGAAATCAAAG | SEQ ID NO: 625 |
| THE1D | CNTCTCTCTCCTGCCGCCTTGTGAAGAAGGTGCTTGCTTCCCCTTTGCCT | SEQ ID NO: 626 |
| LTR5B | CCTCCGTATGCTGAGCGCCGGTCCCCTGGGCCCACTGTTCTTTCTCTATA | SEQ ID NO: 627 |
| MER46 | TTGAGTATCCCTTATCCAAAATGCTTGGGACCAGAAGTGTTTCGGATTTC | SEQ ID NO: 628 |
| CHARLIE4 | GTGACTCCACATGTTAATGGTCTTATTCAAGCTAAGCAGCATCTACTATC | SEQ ID NO: 629 |
| CHARLIE9 | CGTTGCAACGTGCACAGTTCATGCTAAGGATCCGTGCGATGCACTCTGAT | SEQ ID NO: 630 |
| TIGGER8 | NGTCNATTGTTTGACTTTCACACATTCGACTTCCATACACGTTTTCAGGA | SEQ ID NO: 631 |
| MER5A1 | TACTGAATCAGAATCTGCGTTTTAACAAGATCCCCAGGTGATTCATATGC | SEQ ID NO: 632 |
| KANGA2_A | TTGGCCANAAAACTTTTNTTGAATCTTCTCATTGGGAAAATTGGGAGATC | SEQ ID NO: 633 |
| FORDPREFE | TTCACGTGCACTGATTGGACAATAAACAAATACGTAAGTACCTCTTCTCT | SEQ ID NO: 634 |
| CT | | |
| FORDPREFE | ACTTAGAAAATTTCGAGGAAGGCACTCCAAAGCACGGGGTCCCCTGAGGC | SEQ ID NO: 635 |
| CT_A | | |
| LTR16E | ACGCATCACCTTGCATTGCTTCCCATCCTTCCCTGCCTCACTTCCCTTTT | SEQ ID NO: 636 |
| L1PA17_5 | CGAAGCCAAACGATCATACACAACATACACCACAGTCATACCCTCAAGGG | SEQ ID NO: 637 |
| CHARLIE10 | AGTAGCGCTGTCATCAATCCAACCTAGATTAGATAAGTTAACAAGCAAGA | SEQ ID NO: 638 |
| THE1B | CGCCATGATTGTGAGGCCTCCCCAGCCATGTGGAACTGTGAGTCCATTAA | SEQ ID NO: 639 |
| MSTA | ATGATTGTAAGTTTCCTGAGGCCTCCCCAGAAGCCGAGCAGATGCCAGCA | SEQ ID NO: 640 |
| MSTC | ATGCGGCCCCTCGACCTTGGACTTCCCAGCCTCCAGAACTGTAAGAAATA | SEQ ID NO: 641 |
| MLT1A | GCCGTCTACGAACCAGGGAATGAGCCCTCACCAGAAACTGAATCTGCCGG | SEQ ID NO: 642 |
| MLT1B | GCCATCTACAAGCCAAGGAGAGAGGCCTCAGAAGAAACCAACGCTGCCGA | SEQ ID NO: 643 |
| MLT1C | CATGGAACAGATTCTCCCTCACAGCCCTCAGAAGGAACCAACCCTGCCGA | SEQ ID NO: 644 |
| MLT1D | TAGCCCAGTGAGACCCATTTCGGACTTCTGACCTCCAGAACTGTAAGATA | SEQ ID NO: 645 |
| MLT1E | TTGTGAGACCCTGAAGCAGAGGACCCAGCTAAGCTGTGCCCGGACTCCTG | SEQ ID NO: 646 |
| MLT1F | CATCTTGACTGCAACCTCATGAGAGACCCTGAGCCAGAACCACCCAGCTA | SEQ ID NO: 647 |
| MLT2A1 | GTTCTTCAGTTTTGGGACTCGGACTGGCTCTCCTTGCTCCTCAGCTTGCA | SEQ ID NO: 648 |
| MLT2B2 | TCACGTGAGCCAATTCCCCTAATAAATCYCYTCTATCCATCCTATTGGTT | SEQ ID NO: 649 |
| MLT2C2 | CCACAATCGCGTGAGCCAATTCCTTAAAATAAATCTCTCTCTACACACAC | SEQ ID NO: 650 |
| MLT2D | TCTGCCTGCCTGATNGTCTTCGAACTGGAATATCAGCTCTGCGGATTTTG | SEQ ID NO: 651 |
| MER4A | TAAAASCAAGCTGTRCCCCGAGCACCTTGGGCACATGTCGTCAGGACCTC | SEQ ID NO: 652 |
| MER4B | CTAAAATGTATAAAASCAAGCTGTRCCCCGACCACCTTGGGCACATGTKG | SEQ ID NO: 653 |
| MER4C | ATTGAAGCCCTCAAAATCATCTTTGGAGAAAGGCACAGACCACAGATGTT | SEQ ID NO: 654 |
| MER9 | GCTGTGAGACCCCTGATTTCCCACTTCACACCTCTATATTTCTGTGTGTG | SEQ ID NO: 655 |
| MER11A | CACGGTCCTACCGATATGTGATGTCACCCCYGGAGGCCCAGCTGTAAAAT | SEQ ID NO: 656 |
| MER11B | CCGGATRCCCAGCTTTAAAATTTCTCTCTTTTGTACTCTGTCCCTTTATT | SEQ ID NO: 657 |
| MER39 | GGTCTTTGGGTCTTCATTTCTGAAGGCTCCCATGTCACGTAAAACTTTGA | SEQ ID NO: 658 |
| MER48 | TGTTGTTGTGGACGCGCTCTCGGGGTTSGAACCGAYACAAGARCGTTACA | SEQ ID NO: 659 |
| LOR1 | TCTTCCTTGGCAATAMTYRTTGTCTCAGTGATTGGCTTTCTGTGCAGTGA | SEQ ID NO: 660 |
| MER49 | TGCGGGATGGCCACCTTGCAGGCTGTAACCCTTTATAAGAAATAAAGTCT | SEQ ID NO: 661 |
| MER39B | TGCCTTTTCTCCWATTAATCTGCCTTTTGTSAGTTGATTTTTCAGTGAAM | SEQ ID NO: 662 |
| MER61 | AAGCCTAAWTTTTCGTGGCCGTGTGACAAGGACCCCGTCTTTAGCTGAAC | SEQ ID NO: 663 |
| MER31 | CCTGTACCTATCGCAATGGTCCTGAATAAAGTCTGCCTTACCGTGCTTTA | SEQ ID NO: 664 |
| MER34 | GCCGGAAACTCTAAGAGGGTAGAGGWAAAATTTTTCCTTCYCTNCCATGG | SEQ ID NO: 665 |
| MER41C | TTTACACTGTGGAATCACCCTGAATTCTTTCTTGCATGAGATCCAAGAAC | SEQ ID NO: 666 |
| MER50 | TGCTCTAAAACTTGCCTCGGTCTCTTTTTCTGCCTTATGCCCCTCAGTCG | SEQ ID NO: 667 |
| MER65A | GAATATGCACATAGTTTACTATGGCACGCGTATTCCCATTGCAATGCTCT | SEQ ID NO: 668 |
| MER65B | GTGTATGCCCCAAATTGCAATTCTGTTCTTCACATGTTATTCCCAAATAA | SEQ ID NO: 669 |
| MER66A | AGCCGCTTCAATAAAAGTTGCTGTCTAATACCACCARCTCGCCCTTGAAT | SEQ ID NO: 670 |
| MER66B | AGCCGCTTCAATAAAAGTTGCTGTCTAATACCACCARCTCGCCCTTGAAT | SEQ ID NO: 671 |
| MER67A | ATTCTCCCTTTAAAACGCCCAGTCACCTCTGCACAAATCGAAGCTGAGCT | SEQ ID NO: 672 |
| MER67B | CCTCATTCTCCCTTTAAAACGCCCAGTCACCTCTGCACAAATTGGAATGG | SEQ ID NO: 673 |
| MER67C | TAGCAGATTGGCTGTGATGCGCATCACATTCTGGTTTAATGCTTATTCAA | SEQ ID NO: 674 |
| MER68A | CCTGTGAGTCCTCCTAGCGAATCACCGAACCTGGGGGTGGTCTTGGGAAC | SEQ ID NO: 675 |
| MER68B | TTCCCTTTGCTGATCTTGCCGTGTATCCTTACNRTGTCGCTGTAATAAAT | SEQ ID NO: 676 |
| MER70A | TGTTCTGTCTCACCGGACTCAGACAAGTTGGTAACCAGTGCACAGTGAAC | SEQ ID NO: 677 |
| MER70B | TCNGACCCCTATTCCTGGTGGTTGGCATAGTGATGATCTTTGCTATTCTC | SEQ ID NO: 678 |
| MER72 | GCTGCAACCCTTTATGAGAAATAAAGCTCTCCTTTCCAAATTTATGAACC | SEQ ID NO: 679 |
| MER73 | GGTGACGGGGTACGACTGGGTTTCAAACAACTTATGTCAGGCCTAAAAAT | SEQ ID NO: 680 |
| MER74 | AAGCATGATTAATACAAKYTGCTCTGTGATGAACGGATGCCAAATAGWCG | SEQ ID NO: 681 |
| MER76 | TGTTGCCTTAATCGGCTNCTCTGACACCCGGCAGCTCAGCTCTCTCTCCA | SEQ ID NO: 682 |
| MER77 | CTTCTAGCGAATCACTGAACCTGAGGGTGGTCTTGGGGACCCCCGACACA | SEQ ID NO: 683 |
| MLT1G | GCGTCTTGACTGCGCCGATACCACGTGGGACAGAGAWGAACTRCCCAGCT | SEQ ID NO: 684 |
| PABL_A | AATAAAAACTCTCTTCCTCCCCAGTTCATCTGCATCTCGTTATTGGGCCA | SEQ ID NO: 685 |
| PABL_B | CCAGTTCATCTGCATCTCGTTATTGGGCCACGAGAATAAGCAGCCCGACC | SEQ ID NO: 688 |
| MER41D | ATAAACTTGCTCTTCTCACTGTACTCCGCAACTCGCCTTGAATTCCTTCC | SEQ ID NO: 687 |
| MER51A | CTCTGCTTTTGTTGCTTCATTCTTTCCTTGCTTTGTTTGTGCGTTTTGTC | SEQ ID NO: 688 |
| MER51B | CTCTGCTTTTGTTGCTTCATTCTTTCCTTGCTTTGTTTGTGCGTTTTGTC | SEQ ID NO: 689 |
| MER57A | ATCTTCTACCACATGGCTGCACTGGAGTCTCTGAACCTACTCTGGTTCTG | SEQ ID NO: 690 |
| MER57B | TATAAATTTGTTCCGACCACGAGGCATCCCTGGAGTCTCTCTGAATCTGC | SEQ ID NO: 691 |
| AAER65C | ACCTCCAACCTTCTCTTTGTTCTTTGGACATACCGAAGACCACCTGGTCT | SEQ ID NO: 692 |
| MER83 | ACAACTGTCTTGGTAAATTATTTTTACCTCCCGCGCCACCGGCCCCAGAT | SEQ ID NO: 693 |
| MER54 | TGAAAGATACACTGTAAACACCCACAACCAMCTTCCCTGGAGCCCCATCA | SEQ ID NO: 694 |
| MER87 | ACTTACTGGCTGTCGWGCGGTGAGCAGTACCAGCTTTGGATTCAGTTACA | SEQ ID NO: 695 |
| MER74A | AATGGCAGTCGTCTCCTGATCTGTTGGCCTTACCATACCTGAATAATAAT | SEQ ID NO: 696 |
| MER74B | CTTTTCAATGGCAGTCGTCTCCTGATCTGTTGGCCTTACCATACCTSAAT | SEQ ID NO: 697 |
| MER88 | AGGGGAACTTGTGGCAGGGACCAGCCTTATCACACTGGTGCACCTGGTCA | SEQ ID NO: 698 |
| MER54B | AGCCATTTGGGTGTGGTGTAGAACTGGAAACTGTGTCAAGGGTGACTGAG | SEQ ID NO: 699 |
| MER31A | AAATTCCCACTTGCCCATGCTGTATTCGGAGTTGAGCCCAATCTCTCTCC | SEQ ID NO: 700 |
| MER31B | TCCCCACTTGTCCTTGCTGTATTCGGAGTTGAGCCCAATCTCTCTCCCCT | SEQ ID NO: 701 |
| MER67D | ATCCACCTGCCTTTTGTTTCAGNGGAGTTGAGTTCAANCTCTAACCCCTA | SEQ ID NO: 702 |
| MER11C | TTGTACTCTGTCCCTTTATTTCTCAAGCCAGCCGACGCTTAGGGAAAATA | SEQ ID NO: 703 |
| MER11D | ACTATCTTGTGTGTGTCTATTATTTCTCAACCTGCCGATCCGCCTAGGAG | SEQ ID NO: 704 |
| MER61B | CGCCCAATAAATTCTGCTCCTCACCCTTCAATGTGTCCGCGWGCCTAATC | SEQ ID NO: 705 |
| MER61C | GKGACAAGAACCCGGGTTTTAGCTGAACTAAGGAGCAAAATYCTGCAWCA | SEQ ID NO: 706 |
| MER92A | GTTCCTGAGGTCGGAGCGTTCTCCCTATTGCAATAGTCTTTTTGAATAAA | SEQ ID NO: 707 |
| MER92B | TTCTGCCTGAACTTTGAGATGCTTGCAGATCTTATGGTCAGAGCGTTCTC | SEQ ID NO: 708 |
| MER92C | TATCTACCCCTTCCTATAAAAGTCCAAGGCAAAACCACCCTGCCGAGACA | SEQ ID NO: 709 |
| MER93 | CTTCCTCATNCACCYTATAAAAGCCTTTCCTTCAAGCCCCTCCGGCGGAG | SEQ ID NO: 710 |
| MLT1H | CACAGATGCATGAGGGAGCCCAGCCGAGACCAGAAGAACCACCCAGCTGA | SEQ ID NO: 711 |
| MER89 | AAGCTCTGAATAAATAGCCTTTGCTTGTTCTCATTTGGKTGGTCTTCATT | SEQ ID NO: 712 |
| MER90 | CCTCGCTGCARCGAGCAATAAACCCAACTTGTTCAACCACAGGTGTGTTC | SEQ ID NO: 713 |
| MLT2A2 | TGTGGGACTTCACCTTGTGATCGTGTGAGTCAATACTCCTTAATAAACTC | SEQ ID NO: 714 |
| MLT1I | GAGCAGAGCCCCAGCCGACCCGCGATGGACATGTAGCATGAGCAAGAAAT | SEQ ID NO: 715 |
| MER52B | GCCACAGAGGTTTCCGGCCAGAAAAGCGACACCCCAAGGATCCCATGACA | SEQ ID NO: 716 |
| MER52C | ACACTAAATAAAGCTCTTCTTCGTCTTCTTCACCCTTCACTTGTGTGCGT | SEQ ID NO: 717 |
| MER95 | TTGARGTCTCCCGGTTCGCGARCTGTWCTTTCTCTYATTGTATGCACAAT | SEQ ID NO: 718 |
| MLT1J | ATGGAGCAGAGCTGCCATACCAGCCCTGGACTGCCTACCTCTAGACTTCT | SEQ ID NO: 719 |
| MLT1K | AGCTACCCCTGGACTTTTCAGTTACGTGAACCAATAAATTCCCTTTTTTG | SEQ ID NO: 720 |
| MER101 | TTCGTTTTACACCGAAGGCTGCATCTCCCCGGTTTGCAAACTGTTCACTG | SEQ ID NO: 721 |
| MER41E | TTTCTGACTCATCCTTGAATTCCTTCTCGCGATGGTGTCAAGAGCCTGGA | SEQ ID NO: 722 |
| MLT2E | TCCCCCCTCCAGACCTTCACTTCCCCAGCTCCTCCCACAATTGTATAAGG | SEQ ID NO: 723 |
| MLT1E1 | TGATTTCAGCCTTGTGAGACCCTGAGCAGAGGACCCAGCTAAGCCGTGCC | SEQ ID NO: 724 |
| MLT1J1 | AGCCACTGTACATTTTGGGGTTTATTTGTTACAGCAGCTAGCGTTACCTT | SEQ ID NO: 725 |
| MLT1J2 | CCTGAGTCACTACNTGGAGGAGAGCCACCCACACCCGACCAGAACCCNCA | SEQ ID NO: 726 |
| MLT1E2 | TTGATTTCGGCCTTGTGAGACCCTGAGCAGAGAACCCAGCCGAGCCCACC | SEQ ID NO: 727 |
| MLT1G1 | TGCCCAAATTGCAGATTCGTGAGCAAAATAAATGATTGTTGTTGTTTTAA | SEQ ID NO: 728 |
| MER110 | CTCAGCTTTGCTTGATCAACAGGTTTTNTTTTCTGGTGGTCTTTTTGGGG | SEQ ID NO: 729 |
| MER110A | TGGTGCTCYCCCTTACCACAGTAAGCAATAAACTCAGCTTTGTCTTATCA | SEQ ID NO: 730 |
| MLT1F1 | GAGAGACCCTGAGCCAGAACCACCCAGCTAAGCTGCTCCCGAATTCCTGA | SEQ ID NO: 731 |
| MER101B | GGCTGTGTCTCCCTGGTTTGCAAACTGTTCACTGGAATAAACTCTCCTCC | SEQ ID NO: 732 |
| MLT1G2 | CCCTGCTGTGCCCTGTCCGAATTCCTGACCCACAGAATCCGTGAGCATAA | SEQ ID NO: 733 |
| MSTA1 | AGATGCTCGCACCATGCTTTTTGTCCAGCCAGCAGAAYTATGAGCCAAAT | SEQ ID NO: 734 |
| MLT1G3 | AGCCTTCAAGTCTTCCCAGCTGAGGCCCCAGACATCATGGAGCAGAGACA | SEQ ID NO: 735 |
| MSTA2 | TGCCCTTGAACTTCCCAGCCTGCAGAACCATGAGCTAAATAAACCTCTTT | SEQ ID NO: 736 |
| MLT1C1 | GCCTCCAGAGGGAGCATGGCCCTGCTGACACCTTKGATTTCAGCCCAGTG | SEQ ID NO: 737 |
| MSTD | GATGACGCAGCAAGAAGGCCCTCACCAGATGCCGGCNCCWTGATCTTGGA | SEQ ID NO: 738 |
| MER51C | TCTCGCTTTAATAAATTCCTGCTTTCGCTGCTTCGTTCCTGTGTTTCATT | SEQ ID NO: 739 |
| MER21A | TGGTGTGAGAGCAGAGGAAAAACACGGTTTGAGAGAGTTTTCCCGAAACA | SEQ ID NO: 740 |
| MER34B | TCTGTCTTTTGTTACAGGGGTCTATTCCAACTAAGAACTTATGAGGGTTG | SEQ ID NO: 741 |
| MER54A | TATCTGGATCGACCACATTGAGGAACTGGGAGGAGGCGGAGAACTGGAAA | SEQ ID NO: 742 |
| MER74C | GCCTTTCATCTATCCGAGTGTCANTGTGTTGTGTCCCGCCATCAAAAGAA | SEQ ID NO: 743 |
| THE1A | CTCATTTTCCTCTTGCCGCCGCCATGTAAGAAGTGCCTTTCGCCTCCCGC | SEQ ID NO: 744 |
| THE1C | ATGTGAAGAAGGACGTGTTTGCTTCCCCTTCCGCCATGATTGTAAGTTTC | SEQ ID NO: 745 |
| MSTB | ATGATTGNAAGCTTCCTGAGGCCTCACCAGAAGCCGAGCAGATGCCGGCG | SEQ ID NO: 746 |
| MSTB1 | GCCATGCTTCTTGTACAGCCTGCAGAACCGTGAGCCAAATAAACCTCTTT | SEQ ID NO: 747 |
| MER51E | CTGTGGAGTGTACTTTCGCTTCAATAAATCTGTGCTTTCGTTACTNCGTT | SEQ ID NO: 748 |
| MER41F | TGGGTGGCACCACAGTTCCGAGAAATCTTCACCTTTTTCCAGGAATCTTC | SEQ ID NO: 749 |
| MER65D | TAAAAGCTTCCCTTTACCCTCCCCTCTTCAGATGCATCTGTGGCTTGCCA | SEQ ID NO: 750 |
| MER72B | TCCTTTTACCCCTCCCTCAAAGTGCTTTGCTCTCAGCTTCTGCCAGAGGC | SEQ ID NO: 751 |
| MER34C | TTGTTACAGGGGTCTGTCCCAGCTAAGAACTATGAAGGGTAGAGAGAAAA | SEQ ID NO: 752 |
| MER50B | GATATGCCGCYGGTAACTCAGGGTAACTCGGATCTCTTCCACCGGTAACA | SEQ ID NO: 753 |
| MER93B | CTATAAAAGCCTCCCCCTTGCATTCCCTCGGTGGAGCTCCCGAACCACTT | SEQ ID NO: 754 |
| MLT1A1 | CATCTTGGAAGCAGAGASCAGGCCCTCACCAGACACCAAACCTGCTGGNA | SEQ ID NO: 755 |
| MLT1E1A | CTTGTGAGACCCTGAGCAGAGGACCCAGCTAAGCTGTGCCCAGACTCCTG | SEQ ID NO: 756 |
| MER4E1 | TCACGGGCCATGGTCACTCATATTTGGCTCAGAATAAATCTCTTCAAATA | SEQ ID NO: 757 |
| PRIMA4_LTR | TTTAAATTTGGAGCCCTCAAAATCATCTTCGGAGAAAGGCATAGACCTGT | SEQ ID NO: 758 |
| MLT1F2 | ACACCTTGATTGCAGCCTTGTGAGAGACCCTGAGCCAGAAGACCCAACTA | SEQ ID NO: 759 |
| MLT2B3 | CTTCTCAGCCTCCATAATCAAGTGAGCCAATTCCCCTAATAAATCCCTTC | SEQ ID NO: 760 |
| MER66C | GAGCAGTACCGTTCAATAAAAGATTGCTGTCTAACACCACTGGCTCACCC | SEQ ID NO: 761 |
| MER52D | CTCAGGCAAAGGHACCACHGGHCACAGAGGTTTCTGGCCAGAAAAGBGAC | SEQ ID NO: 762 |
| MER41G | TGCTTTGCAATAAAAGCTTCTTGCCTTTCGCTTCATTCTGACTCATCCCT | SEQ ID NO: 763 |
| MER21C | TGTGGGATCTGATGCTAACTCCAGGGTAGATAGTGTCAGAATTGAATTAA | SEQ ID NO: 764 |
| MLT2B4 | CCTGGGTCTCCAGCTTGCCAACTCACCCTGCAGATCTTGGGACTTCTCAG | SEQ ID NO: 765 |
| MER9B | TAAATATGTGGGTCAAACTCTGTTTGTGGCTCTCAGCTCTGAAGGCTGTT | SEQ ID NO: 766 |
| MLT1H2 | TACACCATGTGGAGCAGAAGAACCACCCAGCTGAGCCCAGCCAACACAGA | SEQ ID NO: 767 |
| MER4A1 | AAAACCAAGCTGTGCTCTGACCACCTTGGGCACATGTCGTCAGGACCTCC | SEQ ID NO: 768 |
| MER4D1 | TCANAGGCCATGGTCACTCATATTTGGCTCAGAATAAATCTCTTCAAATA | SEQ ID NO: 769 |
| ThE1D | TGCTTGCTTCCCCTTTGCCTTCTGCCATGATTGTAAGTTTCCTGAGGCCT | SEQ ID NO: 770 |

The expression patterns of the present invention can be evaluated by utilizing high-density expression arrays or microarrays. As defined herein, “microarray” can be a chip, a glass slide or a nylon membrane comprising different types of material, such as, but not limited to, nucleic acids, proteins or tissue sections. By utilizing microarray technology, a plurality of transposable element sequences from transposable element families can be analyzed simultaneously to obtain expression patterns. One of skill in the art can design a microarray chip or glass slide that contains the representative nucleic acid sequences of all of the members of a particular transposable element family or the nucleic acid sequences of select members of a particular transposable element family. An array can also contain the nucleic acid sequences of selected transposable elements from one or more families. Array design will vary depending on the transposable element families and the sequences from these families being analyzed. One of skill in the art will know how to design or select an array that contains the transposable element sequences associated with a particular type of cancer. Such microarrays can be obtained from commercial sources such as Affymetrix, or the microarrays can be synthesized. Methods for synthesizing such arrays containing nucleic acid sequences are known in the art. See, for example, U.S. Pat. No. 6,423,552, U.S. Pat. No. 6,355,432 and U.S. Pat. No. 6,420,169 which are hereby incorporated in their entireties by this reference.

The present invention also provides microarray slides or chips comprising transposable element sequences or fragments thereof from transposable element families. As stated above, a microarray slide or chip can contain the representative nucleic acid sequences of all of the members of one or more transposable element families or the nucleic acid sequences of select members of one or more transposable element families. The present invention also provides for a kit comprising a microarray slide or chip of the present invention for diagnosis of cancer, staging of cancer, other clinical applications and research applications. Utilizing the methods of the present invention, a chip(s) or glass slide(s) that specifically detect a type of cancer can be synthesized. For example, if it is known that transposable element sequences from two families are expressed in prostate cancer, a chip that contains the necessary transposable element sequences from these two families can be synthesized, such that one of skill in the art can utilize a kit, containing this chip, for detecting and staging prostate cancer. Similarly, utilizing the expression patterns of transposable element sequences for breast cancer, it is possible to manufacture a kit containing a chip comprising the transposable element sequences involved in breast cancer in order to diagnose and stage breast cancer. Also, utilizing the expression patterns of transposable element sequences for ovarian cancer, it is possible to manufacture a kit containing a chip comprising the transposable element sequences involved in ovarian cancer in order to diagnose and stage ovarian cancer.

Microarray techniques would be known to one of skill in the art. For example, U.S. Pat. No. 6,410,229 and U.S. Pat. No. 6,344,316, both hereby incorporated by this reference, describe methods of monitoring expression by hybridization to high density nucleic acid arrays. For example, one skilled in the art would first produce fluorescent-labeled cDNAs from mRNAs isolated from cancer cells. A mixture of the labeled cDNAs from the cancer cells is added to an array of oligonucleotides representing a plurality of known transposable elements, as described above, under conditions that result in hybridization of the cDNA to complementary-sequence oligonucleotides in the array. The array is then examined by fluorescence under fluorescence excitation conditions in which transposable element polynucleotides in the array that are hybridized to cDNAs derived from the cancer cells can be detected and quantified.

The expression patterns of the present invention can also be determined by assaying for mRNA transcribed from transposable elements, assaying for proteins expressed from a mRNA, RT-PCR and northern blotting. Particular protein products translated from mRNAs transcribed by transposable element genes can be detected by utilizing immunohistochemical techniques, ELISA, 2-D gels, mass spectrometry, Western blotting, and enzyme assays.

In the present invention, patterns of expression can include one, two, three, four, five, six, seven, eight, nine, ten, twenty or more families of transposable elements and at least one, two, three, four, five, ten, fifteen, twenty, twenty-five, fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members of each transposable element family are being analyzed. For example, the present invention provides for the determination of an expression pattern of one family of transposable elements in which one, two, three, four, five, ten, fifteen, twenty, twenty five, fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members of a transposable element family are analyzed. The present invention also provides for the determination of an expression pattern of two families, wherein one, two, three, four, five, ten, fifteen, twenty, twenty five fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family. Similarly, the invention provides for the determination of an expression pattern of three families, wherein one, two, three, four, five, ten, fifteen, twenty, twenty five fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family. Similarly, the invention provides for the determination of an expression pattern of multiple families, for example, 10, 20, 30, 40, 50, 100, 150, 200, 250, 300, 350, 400, 450, 500, 550, 600, 650 or 700 families wherein one, two, three, four, five, ten, fifteen, twenty, twenty five fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family.

By utilizing the methods of the present invention, a reference expression pattern can be obtained for normal tissues or cells, for particular types of cancers as well as for stages of particular types of cancers. Therefore, the present invention provides a method of assigning an expression pattern of transposable elements to a type of cancerous cell in a sample, comprising: a) determining expression of one or more families of transposable elements; and assigning the expression pattern obtained from step a) to the type of cancerous cell in the sample. The present invention also provides a method of diagnosing cancer comprising: a) determining expression of one or more families of transposable elements in a sample to obtain an expression pattern; b) matching the expression pattern of step a) with a known expression pattern for a type of cancer, and c) diagnosing the type of cancer based on matching of the expression pattern of a) with a known expression pattern for a type of cancer.

In the methods of the present invention, the expression pattern obtained from a sample taken from a subject can be obtained from outside sources, such as a testing laboratory or a commercial source. Therefore, the step of obtaining the expression pattern can be performed by one skilled artisan and the step of comparing the expression pattern can be performed by a second skilled artisan. Thus, the present invention provides a method of diagnosing cancer comprising: a) matching a test transposable element expression pattern with a known expression pattern for a type of cancer; and b) diagnosing the type of cancer based on matching of the test expression pattern with a known expression pattern for a type of cancer.

For example, one of skill in the art can obtain an ovarian tumor cell and determine the expression pattern of one or more transposable element families. By determining which transposable element families are expressed as well as which members of these transposable element families are expressed, one of skill in the art can assign this pattern to an ovarian tumor cell. This can be done for an ovarian tumor cell at different stages of cancer, such that a library of expression patterns are readily available to not only diagnose but stage ovarian cancer. Similarly, this can be done for any type of cancer cell, such as a carcinoma cell, a fibroma cell, a sarcoma cell, a teratoma cell, a blastoma cell, a breast tumor cell of epithelial origin, an ovarian tumor cell of epithelial, stromal or germ cell origin, mixed cell types from a tumor or any other cancer cell. By determining the expression patterns of transposable elements at different stages of cancer, the skilled artisan can determine which transposable element families and which members of these families are involved in cancer and cancer progression.

Such libraries of expression patterns are useful for diagnosis, staging and treatment. For example, a sample can be obtained from a patient or subject in need of diagnosis and assayed for transposable element expression. Once the expression pattern is determined according to the methods of the present invention, this expression pattern can be compared to a library of expression patterns to determine the type of cancer as well as the stage of cancer associated with the expression pattern. Once this is determined, appropriate treatment can be prescribed. In addition to identifying expression patterns for different stages of cancer, the present methods are also useful for identifying expression patterns of cancer cells after therapeutic intervention. For example, a sample can be obtained from a patient or subject undergoing treatment for a cancer such as prostate cancer, lymphoma, skin cancer, GI-tract cancer or any other type of cancer. Expression patterns can be obtained and compared to expression patterns before treatment. In this way, the changes in transposable element expression can be monitored such that one of skill in the art would know which transposable element families as well as which members of each family are affected by the treatment. If improvement is seen in the patient, these improvements can be attributed to changes in transposable element expression. Since the skilled artisan will have reference patterns for a normal tissue or cell, changes in transposable element expression after treatment can be monitored to determine if the treatment results in a transposable element expression pattern that more closely resembles normal or “baseline” expression patterns. Improvements can also be monitored clinically by observing changes in tissue health, cellular changes and changes in the subject's overall health. In this way, one of skill in the art can correlate clinical changes with changes in transposable element expression.

For cancers such as breast cancer and ovarian cancer, once a tissue sample is obtained from a subject, this tissue sample can be compared to a library of tissue samples from many subjects, representing various stages of the cancerous tumor. By comparing the tissue sample to a library of tissue samples with known transposable element expression patterns, one of skill in the art can tailor treatment to the individual needs of the subject. For example, if the expression pattern for the subject matches the expression pattern of a particular stage of cancer that is amenable to treatment with a chemotherapeutic agent, then the subject is a candidate for that treatment. Similarly, one of skill in the art can determine the likelihood that the subject will respond to a particular treatment by determining whether or not the subject's pattern corresponds to patterns obtained for those who have responded to treatment. In this way, treatments can be personalized to maximize the outcome while minimizing unnecessary side effects. The patterns in the libraries utilized for comparison purposes can be grouped by age, medical history or other categories in order to better determine the likelihood of response for subjects. In certain cases, the pattern obtained from the subject may correspond to a pattern for a stage of cancer that does not respond to any available treatment. In cases such as these,.one of skill in the art may determine that treatment may not be advisable because the subject may suffer unnecessarily with little or no likelihood of success.

As mentioned above, one of skill in the art will be able to analyze and interpret the differences in expression. For example, if before treatment, certain families and members of these families are expressed, and after treatment, fewer families and/or members of these families are expressed, it can be said that this particular treatment is effective in reducing expression of these transposable elements, such that the treatment is effective in treating the cancer. In some instances, effective treatments may involve decreasing the expression of certain transposable elements and increasing the expression of others. Therefore, once libraries of expression patterns are established from untreated and treated cancer subjects, one of skill in the art will know whether or not treatment is effective in a particular subject by comparing the expression pattern of a sample from the patient at different stages of treatment, with reference patterns established for the successful treatment of that particular type of cancer. If a treatment is not successful in a particular subject, the skilled artisan will recognize this by noting that the expression pattern is not changing as expected, and other dosages, therapies or treatments can be employed.

Therefore, the present invention also provides a method of determining the effectiveness of an anti-cancer therapeutic in a subject comprising: a) determining expression of one or more families of transposable elements, in a sample obtained from the subject, to obtain a first expression pattern; b) administering an anti-cancer therapeutic to the subject; c) determining expression of one or more families of transposable elements in a sample obtained from the subject after administration of an anti-cancer therapeutic to obtain a second expression pattern; and d) comparing the second expression pattern with the first expression pattern such that if the differences between the expression patterns can be correlated with successful treatment, the anti-cancer therapeutic is an effective anti-cancer therapeutic. The changes observed between expression patterns can vary depending on the type of cancer and the stage of cancer. The changes observed can also vary depending on the size, age, weight and other physiological characteristics of the subject.

In some instances, an effective anti-cancer therapeutic will result in fewer transposable elements being expressed in the second expression pattern as compared to the first expression pattern. In other instances, there may be more transposable elements expressed in the second pattern as compared to the first expression pattern. For example, one of skill in the art can diagnose a cancer utilizing the methods of the present invention and assign a first expression pattern to a sample from a subject. The following example is not meant to be limiting and the numbering of transposable elements appears for illustrative purposes only and not for purposes of identifying any particular retroelement sequences. As an example, the first expression pattern comprises the expression of transposable elements 1, 3, 5, 7, 9 from transposable element family A, the expression of transposable elements 23, 56 and 78 from transposable element family B and the expression of transposable elements 10, 15, 25 from transposable element family C. After administration of an anti-cancer therapeutic, a second expression pattern is obtained. The second expression pattern comprises, for example, the expression of transposable elements 3, 5, 9 from family A, the expression of transposable element 23 from family B and the expression of transposable element 15 from transposable element family C. The skilled artisan, upon comparing the patterns, will determine that the anti-cancer therapeutic is effective in reducing the expression of transposable elements 1 and 7 from family A, transposable elements 56 and 78 from family B, and transposable elements 10 and 25 from transposable element family C. The skilled artisan can continue to monitor changes throughout treatment in order to determine which transposable elements are suppressed or expressed as treatment progresses. One of skill in the art can also compare the expression pattern obtained after treatment to the expression pattern of a normal, non-cancerous cell to determine how the treatment is progressing. If the expression pattern after treatment resembles the expression pattern of a normal cell, the treatment can be said to be successful, however, the expression pattern need not be exactly like the expression pattern of a normal cell in order to deem a treatment effective. In effect, if the changes in transposable element expression after treatment are indicative of progression toward the expression pattern of a normal cell, the treatment can be said to be successful.

Analysis of Methylation Patterns

The present invention also provides methods of assessing the methylation status of transposable element sequences and its role in cancer development and progression. Thus, the present invention also provides methods for the determination of methylation patterns of transposable element sequences. By analyzing global methylation patterns of transposable element sequences and transposable element families, one of skill in the art can assign particular transposable element methylation patterns to types of cancer. Such methylation patterns can be used to diagnose, classify and stage cancer. These transposable element methylation patterns can be used in combination with transposable element expression patterns described herein to diagnose, classify and stage cancer.

Also provided by the present invention is a method of determining a methylation pattern of one or more families of transposable elements genes in a sample comprising determining methylation of one or more families of transposable elements.

In the present invention, methylation patterns can include one, two, three, four, five, six, seven, eight, nine, ten, twenty or more families of transposable elements and at least one, two, three, four, five, ten, fifteen, twenty, twenty-five, fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members of each transposable element family. For example, the present invention provides for the determination of a methylation pattern of one family of transposable elements in which one, two, three, four, five, ten, fifteen, twenty, twenty five, fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members of the transposable element family are analyzed. The present invention also provides for the determination of a methylation pattern of two families, wherein one, two, three, four, five, ten, fifteen, twenty, twenty five, fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family. Similarly, the invention provides for the determination of a methylation pattern of three families, wherein one, two, three, four, five, ten, fifteen, twenty, twenty five fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family. Similarly, the invention provides for the determination of a methylation pattern of multiple families, for example, 10, 20, 30, 40, 50, 100, 150, 200, 250, 300, 350, 400, 450, 500, 550, 600, 650 or 700 families wherein one, two, three, four, five, ten, fifteen, twenty, twenty five fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family.

By utilizing the methods of the present invention, a reference methylation pattern can be obtained for normal tissues or cells, for particular types of cancers as well as for stages of particular types of cancers. Therefore, the present invention provides a method of assigning a methylation pattern of transposable elements to a type of cancerous cell in a sample, comprising: determining the methylation pattern of one or more families of transposable elements; and assigning the methylation pattern obtained from step a) to the type of cancerous cell in the sample.

The present invention also provides a method of diagnosing cancer comprising: a) determining the methylation pattern of one or more families of transposable elements in a sample to obtain a methylation pattern; b) matching the methylation pattern of step a) with a known methylation pattern for a type of cancer; and c) diagnosing the type of cancer based on matching of the methylation pattern of a) with a known methylation pattern for a type of cancer.

In the methods of the present invention, the methylation pattern obtained from a sample taken from a subject can be obtained from outside sources, such as a testing laboratory or a commercial source. Therefore, the step of obtaining the methylation pattern can be performed by one skilled artisan and the step of comparing the methylation pattern can be performed by a second skilled artisan. Thus, the present invention provides a method of diagnosing cancer comprising: a) matching a test transposable element methylation pattern with a known methylation pattern for a type of cancer; and b) diagnosing the type of cancer based on matching of the test methylation pattern with a known methylation pattern for a type of cancer.

For example, one of skill in the art can obtain an ovarian cancer sample and determine the methylation pattern of one or more transposable element families. By determining which transposable element families are methylated as well as which members of these transposable element families are methylated, one of skill in the art can assign this methylation pattern to an ovarian cancer sample. This can be done for ovarian cancer samples at different stages of cancer, such that a library of methylation patterns are readily available to not only diagnose but stage ovarian cancer. Similarly, this can be done for any type of cancer cell, such as a carcinoma cell, a fibroma cell, a sarcoma cell, a teratoma cell, a blastoma cell, a breast tumor cell of epithelial origin, an ovarian tumor cell of epithelial, stromal or germ cell origin, mixed cell types from a tumor or any other cancer cell. By determining the methylation patterns of transposable elements at different stages of cancer, the skilled artisan can determine which transposable element families and which members of these families are involved in cancer and cancer progression based on changes in DNA methylation (and/or chromatin structure).

Such libraries of expression patterns are useful for diagnosis, staging and treatment. For example, a sample can be obtained from a patient or subject in need of diagnosis and assayed for transposable element methylation. Once the methylation pattern is determined according to the methods of the present invention, this methylation pattern can be compared to a library of methylation patterns to determine the type of cancer as well as the stage of cancer associated with the methylation pattern. Once this is determined, appropriate treatment can be prescribed. In addition to identifying methylation patterns for different stages of cancer, the present methods are also useful for identifying methylation patterns of cancer cells after therapeutic intervention. For example, a sample can be obtained from a patient or subject undergoing treatment for a cancer such as prostate cancer, lymphoma, skin cancer, GI-tract cancer or any other type of cancer. Methylation patterns can be obtained and compared to methylation patterns before treatment. In this way, the changes in transposable element methylation can be monitored such that one of skill in the art would know which transposable element families as well as which members of each family are affected by the treatment. If improvement is seen in the patient, these improvements can be attributed to changes in transposable element methylation. Since the skilled artisan will have reference patterns for a normal tissue or cell, changes in transposable element methylation after treatment can be monitored to determine if the treatment results in a transposable element methylation pattern that more closely resembles normal or “baseline” methylation patterns. Improvements can also be monitored clinically by observing changes in tissue health, cellular changes and changes in the subject's overall health. In this way, one of skill in the art can correlate clinical changes with changes in transposable element methylation.

For cancers such as breast cancer and ovarian cancer, once a tissue sample is obtained from a subject, this tissue sample can be compared to a library of tissue samples from many subjects, representing various stages of the cancerous tumor. By comparing the tissue sample to a library of tissue samples with known transposable element methylation patterns, one of skill in the art can tailor treatment to the individual needs of the subject. For example, if the methylation pattern for the subject matches the methylation pattern of a particular stage of cancer that is amenable to treatment with a chemotherapeutic agent, then the subject is a candidate for that treatment. Similarly, one of skill in the art can determine the likelihood that the subject will respond to a particular treatment by determining whether or not the subject's pattern corresponds to patterns obtained for those who have responded to treatment. In this way, treatments can be personalized to maximize the outcome while minimizing unnecessary side effects. The patterns in the libraries utilized for comparison purposes can be grouped by age, medical history or other categories in order to better determine the likelihood of response for subjects. In certain cases, the pattern obtained from the subject may correspond to a pattern for a stage of cancer that does not respond to any available treatment. In cases, such as these, one of skill in the art may determine that treatment may not be advisable because the subject may suffer unnecessarily with little or no likelihood of success.

One of skill in the art will be able to assess the differences in methylation. For example, if before treatment, certain families and members of these families are methylated, and after treatment, more families and/or members of these families are methylated, it can be said that this particular treatment is effective in suppressing transposable element methylation such that the treatment is effective in treating the cancer. In some instances, effective treatments may involve decreasing the methylation of certain transposable elements and increasing the methylation of others. Therefore, once libraries of methylation patterns are established from untreated and treated cancer subjects, one of skill in the art will know whether or not treatment is effective in a particular subject by comparing the methylation pattern of a sample from the patient at different stages of treatment, with reference patterns established for the successful treatment of that particular type of cancer. If a treatment is not successful in a particular subject, the skilled artisan will recognize this by noting that the methylation pattern is not changing as expected, i.e., the methylation pattern is not changing such that the methylation pattern more closely resembles the methylation pattern of a noncancerous or successfully treated cancer cell, and other dosages, therapies or treatments can be employed.

Therefore, the present invention also provides a method of determining the effectiveness of an anti-cancer therapeutic in a subject comprising: a) determining the methylation pattern of one or more families of transposable elements, in a sample obtained from the subject, to obtain a first methylation pattern; b) administering an anti-cancer therapeutic to the subject; c) determining the methylation pattern of one or more families of transposable elements in a sample obtained from the subject after administration of an anti-cancer therapeutic to obtain a second methylation pattern; and d) comparing the second methylation pattern with the first methylation pattern such that if the differences between the methylation patterns can be correlated with successful treatment, the anti-cancer therapeutic is an effective anti-cancer therapeutic. The changes observed between methylation patterns can vary depending on the type of cancer and the stage of cancer. The changes in methylation patterns can also vary based on the size, age, weight and other physiological characteristics of the subject.

In some instances, an effective anti-cancer therapeutic will result in fewer transposable elements being methylated in the second methylation pattern as compared to the first methylation pattern. In other instances, there may be more transposable elements methylated in the second pattern as compared to the first methylation pattern. For example, one of skill in the art can diagnose a cancer utilizing the methods of the present invention and assign a first methylation pattern to a sample from a subject. The following example is not meant to be limiting and the numbering of transposable elements appears for illustrative purposes only and not for purposes of identifying any particular retroelement sequences. As an example, this first methylation pattern comprises the methylation of transposable elements 2, 4, 6, 8 and 10 from transposable element family A, the methylation of transposable elements 24, 57 and 79 from transposable element family B and the methylation of transposable elements 11, 16, and 26 from transposable element family C. After administration of an anti-cancer therapeutic, a second methylation pattern is obtained. The second expression pattern comprises, for example, the methylation of transposable elements 2, 4, 6, 8, 10, 12 and 14 from family A, the methylation of transposable element 24, 57, 79 and 80 from family B and the methylation of transposable elements 11, 16, 26 and 32 from transposable element family C. The skilled artisan, upon comparing the patterns, will determine that the anti-cancer therapeutic results in the methylation of transposable elements 12 and 14 from family A, transposable element 80 from family B, and transposable element 32 from transposable element family C. This second methylation pattern can be compared to the methylation pattern of a normal cell to see if the treatment is progressing toward a methylation pattern associated with a non-cancerous cell. This second methylation pattern can also be compared to methylation patterns for different stages of the particular cancer being treated in order to determine if this pattern corresponds to an improvement or a deterioration in the subject's condition. The skilled artisan can continue to monitor changes throughout treatment in order to determine which transposable elements are methylated or non-methylated, and whether or not an improvement can be correlated to changes in methylation, as treatment progresses.

As stated above, the methylation state of non-cancerous cells can serve as a guide to one of skill in the art in determining the effectiveness of a treatment. One of skill in the art can compare the methylation pattern obtained after treatment to the methylation pattern of a normal, non-cancerous cell to determine how the treatment is progressing. If the methylation pattern after treatment resembles the methylation pattern of a normal cell, the treatment can be said to be successful, however, the methylation pattern need not be exactly like the methylation pattern of a normal cell in order to deem a treatment effective. In other words, if the changes in transposable element sequence methylation after treatment are indicative of progression toward the methylation pattern of a normal cell, the treatment can be said to be successful.

The methylation patterns of the present invention can be correlated to transposable element expression patterns and/or chromatin status patterns described herein, such that one of skill in the art, upon obtaining a particular expression pattern and/or a chromatin status pattern, will also know what the methylation status of the sample is. Also, upon obtaining upon obtaining a particular methylation pattern, one of skill in the art will also know the expression pattern and/or chromatin status of the sample.

Methods of measuring methylation are known in the art and include, but are not limited to methylation-specific PCR, methylation microarray analysis and ChIP (a chromatin immunoprecipitation approach) analysis. Methylation can also be monitored by digestion of nucleic acid sequences with methylation sensitive and non-sensitive restriction enzymes followed by Southern blotting or PCR analysis of the restriction products (See Takai et al. “Hypomethylation of LINE1 retrotransposon in human hepatocellular carcinomas, but not in surrounding liver cirrhosis” Jpn J. Clin. Oncol. 30(7) 306-309). One of skill in the art could also utilize methods in which genomic DNA is digested followed by PCR. (See, for example, Cartwright et al., “Analysis of Drosophila chromatin structure in vivo” Methods in Enzymology, Vol. 304)

Methylation-specific PCR (MSP) technology utilizes the fact that DNA in humans is methylated mainly at certain cytosines located 5′ to guanosine. This occurs especially in, GC-rich regions, known as CpG islands. To distinguish the methylation state of a sequence, MSP relies on differential chemical modification of cytosine residues in DNA. Treatment with sodium bisulfite converts unmethylated cytosine residues into uracil, leaving the methylated cytosines unchanged. This modification thus creates different DNA sequences for methylated and unmethylated DNA. PCR primers can then be designed so as to distinguish between these different sequences. Two sets of primers (and additional control sets of primers) are designed: one set with sequences annealing to unchanged (methylated in the genomic DNA) cytosines and the other set with sequences annealing to the altered (unmethylated in the genomic DNA) cytosines. A comparison of PCR results using the two sets of primers reveals the methylation state of a PCR product. If the primer set with the altered sequence gives a PCR product, then the indicated cytosine was unmethylated. If the primer set with the unchanged sequence gives a PCR product, then the cytosines were methylated and thus protected from alteration. Evron et al. (“Detection of breast cancer cells in ductal lavage fluid by methylation-specific PCR,” Lancet 2001, 357: 1335-1336) describes the use of MSP to detect breast cancer and is hereby incorporated in its entirety by this reference.

To use a microarray to study transposable element methylation, one of skill in the art would select for methylated and unmethylated DNA from total genomic DNA. The selectively isolated DNA is then hybridized to the transposable element array either directly or after amplification and patterns between various cell types / tissue types as described earlier in the patent application.

There are several approaches for selecting methylated DNA. One method is chromatin immunoprecipitation (CHIP ). Another method utilizes a column binding approach and a third method involves ligation of adapters to fragmented genomic DNA and methylation-specific restriction digestion of the ligation products followed by PCR amplification.

In all cases, the selected DNA fragments are labeled by incorporation of dNTPs coupled with fluorescent dyes (for example Cy3 or Cy5 coupled dNTPs) and hybridization to the microarray is performed according to standard protocols. One of skill in the art could utilize the BioPrime DNA labeling system from Life Technologies or other kits available for such labeling.

As stated above, microarray techniques would be known to one of skill in the art. For example, U.S. Pat. No. 6,410,229 and U.S. Pat. No. 6,344,316, both hereby incorporated by this reference, describe methods of hybridizing nucleic acids to high density nucleic acid arrays. For example, one skilled in the art would first produce fluorescent-labeled DNA isolated from the tissue of interest. A batch of labeled genomic/amplified genomic DNAs representing either one sample or a mixture of two samples from the tissue sources of interest is added to an array of oligonucleotides representing a plurality of known transposable elements, as described above, under conditions that result in hybridization of the DNAs to complementary-sequence oligonucleotides in the array. The array is then examined by fluorescence under fluorescence excitation conditions in which transposable element oligonucleotides in the array that are hybridized to genomic/amplified genomic DNAs derived from the tissue of interest can be detected and quantified.

ChIP technology involves in vivo formaldehyde cross-linking of DNA and associated proteins in intact cells, followed by selective immunoprecipitation of protein-DNA complexes with specific antibodies. Such an approach allows detection of any protein at its in vivo binding site directly. In particular, proteins that are not bound directly to DNA or that depend on other proteins for binding activity in vivo can be analyzed by this method. Since methylation involves methylation complexes that involve numerous proteins which interact with DNA, by utilizing CHIP technology, methylation complexes can be cross-linked to transposable element sequences to which they are bound and then an antibody specific to one of the proteins (i.e., one of the proteins involved in the methylation complex, such as methyltransferase or a protein having a methyl binding site, for example, MBD1) can be utilized to immunoprecipitate the methylation complex-DNA bound sequence. The complex can then be chemically released and the transposable element sequence to which it was bound can be identified. For references describing ChIP technology, see Orlando (“Mapping chromosomal proteins in vivo by formaldehyde crosslinked-chromatin immunoprecipitation,” TIBS 2000, 25:99-104) and Kuo et al. (“In Vivo Cross-Linking and Immunoprecipitation for Studying Dynamic Protein:DNA Associations in a Chromatin Environment,” 1999, 19: 425-433) both of which are incorporated in their entireties by this reference.

The column binding approach is used to select for methylated DNA after genomic DNA extraction. The column contains methyl-CpG-binding proteins, for example the methyl-binding domain of rat MeCP2, covalently linked to a histidine tag, then attached to a Ni-agarose matrix. Fragmented genomic DNA (digested with restriction enzymes, for example Mse1) is run through the column. The column retains DNA containing methylated cytosines, unmethylated DNA is collected from the flow-through. Retained methylated DNA is recovered from the column. (Cross, S. H., Charlton, J. A., Nan, X. and Bird, A. P. (1994) Purification of CpG islands using a methylated DNA binding column. Nat Genet., 6, 236-244 and Brock, Huang, Chen and Johnson (2001) A novel technique for the identification of CpG islands exhibiting altered methylation patterns (ICEAMP). Nucleic Acids Research, vol. 29, no. 24). The isolated DNA can be ligated to linker oligonucleotides and amplified by PCR. Fluorescence labeling and hybridization is then performed as described above.

Formaldehyde crosslinking followed by chromatin immunoprecipitation is reviewed in Orlando 2000. By addition of formaldehyde to live tissue/cells, DNA and nearby proteins are cross-linked in vivo, followed by sonication of the tissue/cell suspension. The DNA is fragmented in the process. Antibodies recognizing methyl-binding proteins are added and the immune complexes are collected, thereby precipitating methylated DNA with associated proteins. DNA without methyl-binding proteins will be collected from the supernatant. The cross-linking step is then reversed for both fractions, followed by a DNA purification step. The isolated DNA can be ligated to linker oligonucleotides and amplified by PCP, Fluorescence labeling and hybridization is then performed as described above.

Linker ligation/Methylation-specific restriction/ PCR can also be utilized. The methods of the present invention can utilize a modified version of DMH (Differential Methylation Hybridization) (References: Huang et al. ‘Methylation profiling of CpG islands in human breast cancer cells’ Human Molecular Genetics 1999, Vol. 8, No. 3 and Yan et al. ‘Dissecting complex epigenetic alterations in breast cancer using CpG island microarrays’ Cancer Research 2001, 61, 8375-8380). Genomic DNA is digested with MseI. Then, the ends of the resulting fragments are ligated to linker oligonucleotides. Ligated fragments undergo restriction digestion with methylation-sensitive enzymes BstUI and/or HpaII, followed by PCR amplification of undigested fragments. Fluorescence labeling and hybridization is then performed as described above.

A COT-1 subtractive hybridization step can be utilized at some point before labeling the DNA to separate out the highly repetitive sequences from the sample (See Craig et al. ‘Removal of repetitive sequences from FISH probes using PCR-assisted affinity chromatography’ Human Genetics 1997, Vol. 100, 472-476).

Another technique, methylation-specific oligonucleotide (MSO) microarray, uses bisulfite-modified DNA as a template for PCR amplification, resulting in conversion of unmethylated cytosine, but not methylated cytosine, into thymine within CpG islands of interest. The amplified product, therefore, may contain a pool of DNA fragments with altered nucleotide sequences due to differential methylation status. A test sample is hybridized to a set of oligonucleotide arrays that discriminate between methylated and unmethylated cytosine at specific nucleotide positions, and quantitative differences in hybridization are determined by fluorescence analysis. For examples of methylation micro array techniques see Gitan et al. (“Methylation-specific oligonucleotide microarray: a new potential for high-throughput methylation analysis,” Genome Res. 2002, 12: 158-164.), Shi et al. (“Oligonucleotide-based microarray for DNA methylation analysis: Principles and applications,” J. Cell Biochem. 2003, 88: 138-143), Yan et al. (“Applications of CpG island microarrays for high-throughput analysis of DNA methylation,” J. Nutr. 2002, 132: 2430S-2434S), Wei et al. (“Methylation microarray analysis of late-stage ovarian carcinomas distinguishes progression-free survival in patients and identifies candidate epigenetic markers,” Clin Cancer Res. 2002, 8: 2246-2252), all of which are incorporated herein, in their entireties, by this reference.

Analysis of Chromatin Status

The present invention also provides methods of assessing the chromatin status of transposable element sequences and its role in cancer development and progression. Thus, the present invention also provides methods for the determination of chromatin status patterns of transposable element sequences. By analyzing global chromatin status patterns of transposable element sequences and transposable element families, one of skill in the art can assign particular transposable element chromatin status patterns to types of cancer. Such chromatin status patterns can be used to diagnose, classify and stage cancer. These transposable element chromatin status patterns can be used in combination with transposable element expression patterns and/or methylation patterns described herein to diagnose, classify and stage cancer.

One of the skill in the art would know how to assess chromatin status by methods standard in the art. See Orlando (“sapping chromosomal proteins in vivo by formaldehyde crosslinked-chromatin immunoprecipitation,” TIBS 2000, 25:99-104) and Kuo et al. (“In Vivo Cross-Linking and Immunoprecipitation for Studying Dynamic Protein:DNA Associations in a Chromatin Environment,” 1999, 19: 425-433) both of which are incorporated in their entireties by this reference.

As utilized herein, “chromatin status” refers to the chromosomal structure or the chromosomal accessibility or the ability of restriction enzymes to access a transposable element sequence or a fragment thereof Therefore, chromatin status patterns can contain sequences that are accessible to restriction enzymes and sequences that are not accessible to restriction enzymes.

Also provided by the present invention is a method of determining a chromatin status pattern of one or more families of transposable element genes in a sample comprising determining chromatin status of one or more families of transposable elements.

In the present invention, chromatin status patterns can include one, two, three, four, five, six, seven, eight, nine, ten, twenty or more families of transposable elements and at least one, two, three, four, five, ten, fifteen, twenty, twenty-five, fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members of each transposable element family. For example, the present invention provides for the determination of a chromatin status pattern of one family of transposable elements in which one, two, three, four, five, ten, fifteen, twenty, twenty five fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members of the transposable element family are analyzed. The present invention also provides for the determination of a chromatin status pattern of two families, wherein one, two, three, four, five, ten, fifteen, twenty, twenty five fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family. Similarly, the invention provides for the determination of a chromatin status pattern of three families, wherein one, two, three, four, five, ten, fifteen, twenty, twenty five fifty, one hundred, two hundred, three hundred, four hundred, five hundred members, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family. Similarly, the invention provides for the determination of a chromatin status pattern of multiple families, for example, 10, 20, 30, 40, 50, 100, 150, 200, 250, 300, 350, 400, 450, 500, 550, 600, 650 or 700 families wherein one, two, three, four, five, ten, fifteen, twenty, twenty five fifty, one hundred, two hundred, three hundred, four hundred, five hundred, one thousand, two thousand, three thousand, four thousand, five thousand, six thousand, seven thousand, eight thousand, nine thousand, ten thousand, twenty thousand, fifty thousand, one hundred thousand, two hundred thousand, three hundred thousand, four hundred thousand or five hundred thousand members are analyzed for each family.

By utilizing the methods of the present invention, a reference chromatin status pattern can be obtained for normal tissues or cells, for particular types of cancers as well as for stages of particular types of cancers. Therefore, the present invention provides a method of assigning a chromatin status pattern of transposable elements to a type of cancerous cell in a sample, comprising: determining the chromatin status pattern of one or more families of transposable elements; and assigning the chromatin status pattern obtained from step a) to the type of cancerous cell in the sample.

The present invention also provides a method of diagnosing cancer comprising: a) determining the chromatin status pattern of one or more families of transposable elements in a sample to obtain a chromatin status pattern; b) matching the chromatin status pattern of step a) with a known chromatin status pattern for a type of cancer; and c) diagnosing the type of cancer based on matching of the chromatin status pattern of a) with a known chromatin status pattern for a type of cancer.

In the methods of the present invention, the chromatin status pattern obtained from a sample taken from a subject can be obtained from outside sources, such as a testing laboratory or a commercial source. Therefore, the step of obtaining the chromatin status pattern can be performed by one skilled artisan and the step of comparing the chromatin status pattern can be performed by a second skilled artisan. Thus, the present invention provides a method of diagnosing cancer comprising: a) matching a test transposable element chromatin status pattern with a known chromatin status pattern for a type of cancer; and b) diagnosing the type of cancer based on matching of the test chromatin status pattern with a known chromatin status pattern for a type of cancer.

For example, one of skill in the art can obtain an ovarian cancer sample and determine the chromatin status pattern of one or more transposable element families. By determining the chromosomal accessibility of transposable element families as well as the chromosomal accessibility of members of these transposable element families, one of skill in the art can assign this chromatin status pattern to an ovarian cancer sample. This can be done for ovarian cancer samples at different stages of cancer, such that a library of chromatin status patterns are readily available to not only diagnose but stage ovarian cancer. Similarly, this can be done for any type of cancer cell, such as a carcinoma cell, a fibroma cell, a sarcoma cell, a teratoma cell, a blastoma cell, a breast tumor cell of epithelial origin, an ovarian tumor cell of epithelial, stromal or germ cell origin, mixed cell types from a tumor or any other cancer cell. By determining the chromatin status patterns of transposable elements at different stages of cancer, the skilled artisan can determine which transposable element families and which members of these families are involved in cancer and cancer progression based on changes in chromatin structure.

Such libraries of expression patterns are useful for diagnosis, staging and treatment. For example, a sample can be obtained from a patient or subject in need of diagnosis and assayed for chromatin status. Once the chromatin status pattern is determined according to the methods of the present invention, this chromatin status pattern can be compared to a library of chromatin status patterns to determine the type of cancer as well as the stage of cancer associated with the chromatin pattern. Once this is determined, appropriate treatment can be prescribed. In addition to identifying chromatin status patterns for different stages of cancer, the present methods are also useful for identifying chromatin status patterns of cancer cells after therapeutic intervention. For example, a sample can be obtained from a patient or subject undergoing treatment for a cancer such as prostate cancer, lymphoma, skin cancer, GI-tract cancer or any other type of cancer. Chromatin status patterns can be obtained and compared to chromatin status patterns before treatment. In this way, the changes in transposable element chromatin status can be monitored such that one of skill in the art would know which transposable element families as well as which members of each family are affected by the treatment. If improvement is seen in the patient, these improvements can be attributed to changes in transposable element chromatin status. Since the skilled artisan will have reference patterns for a normal tissue or cell, changes in transposable element chromatin status after treatment can be monitored to determine if the treatment results in a transposable element chromatin status pattern that more closely resembles normal or “baseline” chromatin status patterns. Improvements can also be monitored clinically by observing changes in tissue health, cellular changes and changes in the subject's overall health. In this way, one of skill in the art can correlate clinical changes with changes in transposable element chromatin status.

For cancers such as breast cancer and ovarian cancer, once a tissue sample is obtained from a subject, this tissue sample can be compared to a library of tissue samples from many subjects, representing various stages of the cancerous tumor. By comparing the tissue sample to a library of tissue samples with known transposable element chromatin status patterns, one of skill in the art can tailor treatment to the individual needs of the subject. For example, if the chromatin status pattern for the subject matches the chromatin status pattern of a particular stage of cancer that is amenable to treatment with a chemotherapeutic agent, then the subject is a candidate for that treatment. Similarly, one of skill in the art can determine the likelihood that the subject will respond to a particular treatment by determining whether or not the subject's pattern corresponds to patterns obtained for those who have responded to treatment. In this way, treatments can be personalized to maximize the outcome while minimizing unnecessary side effects. The patterns in the libraries utilized for comparison purposes can be grouped by age, medical history or other categories in order to better determine the likelihood of response for subjects. In certain cases, the pattern obtained from the subject may correspond to a pattern for a stage of cancer that does not respond to any available treatment. In cases, such as these, one of skill in the art may determine that treatment may not be advisable because the subject may suffer unnecessarily with little or no likelihood of success.

In some instances, effective treatments may involve decreasing the chromatin accessibility of certain transposable elements and increasing the chromatin accessibility of others. Therefore, once libraries of chromatin status patterns are established from untreated and treated cancer subjects, one of skill in the art will know whether or not treatment is effective in a particular subject by comparing the chromatin status pattern of a sample from the patient at different stages of treatment, with reference patterns established for the successful treatment of that particular type of cancer. If a treatment is not successful in a particular subject, the skilled artisan will recognize this by noting that the chromatin status pattern is not changing as expected, i.e., the chromatin status pattern is not changing such that the chromatin status pattern more closely resembles the chromatin status pattern of a non-cancerous or successfully treated cancer cell, and other dosages, therapies or treatments can be employed.

Therefore, the present invention also provides a method of determining the effectiveness of an anti-cancer therapeutic in a subject comprising: a) determining the chromatin status pattern of one or-more families of transposable elements, in a sample obtained from the subject, to obtain a first chromatin status pattern; b) administering an anti-cancer therapeutic to the subject; c) determining the chromatin status pattern of one or more families of transposable elements in a sample obtained from the subject after administration of an anti-cancer therapeutic to obtain a second chromatin status pattern; and d) comparing the second chromatin status pattern with the first chromatin status pattern such that if the differences between the chromatin status patterns can be correlated with successful treatment, the anti-cancer therapeutic is an effective anti-cancer therapeutic. The changes observed between chromatin status patterns can vary depending on the type of cancer and the stage of cancer. The changes in chromatin status patterns can also vary based on the size, age, weight and other physiological characteristics of the subject.

In some instances, an effective anti-cancer therapeutic will result in fewer transposable elements being accessible to restriction enzymes in the second chromatin status pattern as compared to the first chromatin status pattern. In other instances, there may be more transposable elements accessible to restriction enzymes in the second pattern as compared to the first chromatin status pattern. For example, one of skill in the art can diagnose a cancer utilizing the methods of the present invention and assign a first chromatin status pattern to a sample from a subject. The following example is not meant to be limiting and the numbering of transposable elements appears for illustrative purposes only and not for purposes of identifying any particular transposable element-sequences. As an example, this first chromatin status pattern comprises the chromatin status of transposable elements 2 (accessible), 4 (not accessible), 6 (accessible), 8 (not accessible) and 10 (not accessible) from transposable element family A, the chromatin status of transposable elements 24 (not accessible), 57 (accessible) and 79 (not accessible) from transposable element family B and the chromatin status of transposable elements 11 (not accessible), 16 (accessible), and 26 (not accessible) from transposable element family C. After administration of an anti-cancer therapeutic, a second chromatin status pattern is obtained. The second chromatin status pattern comprises, for example, the chromatin status of transposable elements 2 (not accessible), 4 (not accessible), 6 (accessible), 8 (not accessible) and 10 (not accessible) from family A, the chromatin status of transposable element 24 (not accessible), 57 (not accessible) and 79 (accessible) from family B and the chromatin status of transposable elements 11 (accessible), 16 (not accessible) and 26 (not accessible) from transposable element family C. The skilled artisan, upon comparing the patterns, will determine that the anti-cancer therapeutic results in changes in the chromatin status of transposable element 2 from family A, transposable elements 57 and 79 from family B, and transposable element 11 from transposable element family C. This second chromatin status pattern can be compared to the chromatin status pattern of a normal cell to see if the treatment is progressing toward a chromatin status pattern associated with a non-cancerous cell. This second chromatin status pattern can also be compared to chromatin status patterns for different stages of the particular cancer being treated in order to determine if this pattern corresponds to an improvement or a deterioration in the subject's condition. The skilled artisan can continue to monitor changes throughout treatment in order to determine which transposable elements are accessible or not accessible and whether or not an improvement can be correlated to changes in chromatin status, as treatment progresses.

As stated above, the chromatin status state of non-cancerous cells can serve as a guide to one of skill in the art in determining the effectiveness of a treatment. One of skill in the art can compare the chromatin status pattern obtained after treatment to the chromatin status pattern of a normal, non-cancerous cell to determine how the treatment is progressing. If the chromatin status pattern after treatment resembles the chromatin status pattern of a normal cell, the treatment can be said to be successful, however, the chromatin status pattern need not be exactly like the chromatin status pattern of a normal cell in order to deem a treatment effective. In other words, if the changes in transposable element sequence chromatin status after treatment are indicative of progression toward the chromatin status pattern of a normal cell, the treatment can be said to be successful.

The chromatin status patterns of the present invention can be correlated to transposable element expression patterns and/or methylation patterns described herein, such that one of skill in the art, upon obtaining a particular expression pattern and/or methylation pattern, will also know what the chromatin status of the sample is. Also, upon obtaining a particular chromatin status pattern, one of skill in the art will also know the expression pattern and/or methylation pattern of the sample.

The methods of the present invention can also be utilized to differentiate between subtypes of cancers. For example, mantle cell lymphoma and grades I/II follicular lymphoma are subtypes of non-Hodgkin's lymphoma. Similarly, adenocarcinoma, large cell carcinoma, spindle cell carcinoma, squamous cell carcinoma, adenosquamous carcinoma and small cell carcinoma are all subtypes of lung cancer. Numerous subtypes for other cancers are also known and they can be differentiated by the methods of the present invention. By utilizing the expression patterns, chromatin status patterns and/or methylation patterns of cells associated with these subtypes, the skilled artisan can make a more accurate diagnosis of a particular type of cancer. The differences in the expression patterns, chromatin status and methylation patterns of the transposable element sequences allows the skilled artisan to differentiate between subtypes and thus better stage the cancer as well as administer treatment best suited for a specific cancer subtype.

The present invention also provides a computer system comprising a) a database including records comprising a plurality of reference retroelement expression patterns, and associated diagnosis and therapy data; and b) a user interface capable of receiving a selection of one or more test retroelement expression patterns for use in determining matches between a test retroelement expression pattern and a reference retroelement expression pattern, and displaying the records associated with matching expression patterns. The computer systems of the present invention can also include a database including records comprising a plurality of reference methylation patterns, and associated diagnosis and therapy data, b) a user interface capable of receiving a selection of one or more test methylation patterns for use in determining matches between a test methylation pattern and the reference methylation pattern, and displaying the records associated with matching expression patterns. Also provided is a computer system comprising a) a database including records comprising a plurality of reference chromatin status patterns, and associated diagnosis and therapy data; and b) a user interface capable of receiving a selection of one or more test chromatin status patterns for use in determining matches between a test chromatin status pattern and a reference chromatin status pattern, and displaying the records associated with matching expression patterns.

It will be appreciated by those skilled in the art that expression patterns, methylation patterns and/or chromatin status patterns identified from subjects can be stored, recorded, and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate a list of sequences comprising one or more of the nucleic acids of the invention. Another aspect of the present invention is a computer readable medium having recorded thereon at least 2, 5, 10, 15, 20, 25, 30, 50, 100, 200, 250, 300, 400, 500, 1000, 2000, 3000, 4000 or 5000 expression patterns, methylation patterns and/or chromatin status patterns of the invention or patterns identified from subjects.

Computer readable media include magnetically readable media, optically readable media, electronically readable media and magnetic/optical media. For example, the computer readable media may be a hard disc, a floppy disc, a magnetic tape, CD-ROM, DVD, RAM, or ROM as well as other types of other-media known to those skilled in the art.

Embodiments of the present invention include systems, particularly computer systems which contain the sequence information described herein. As used herein, “a computer system” refers to the hardware components, software components, and data storage components used to store and/or analyze the expression patterns of the present invention or other expression patterns. The computer system preferably includes the computer readable media described above, and a processor for accessing and manipulating the data.

Preferably, the computer is a general purpose system that comprises a central processing unit (CPU), one or more data storage components for storing data, and one or more data retrieving devices for retrieving the data stored on the data storage components. A skilled artisan can readily appreciate that any one of the currently available computer systems are suitable.

In one particular embodiment, the computer system includes a processor connected to a bus which is connected to a main memory, preferably implemented as RAM, and one or more data storage devices, such as a hard drive and/or other computer readable media having data recorded thereon. In some embodiments, the computer system further includes one or more data retrieving devices for reading the data stored on the data storage components. The data retrieving device may represent, for example, a floppy disk drive, a compact disk drive, a magnetic tape drive, a hard disk drive, a CD-ROM drive, a DVD drive, etc. In some embodiments, the data storage component is a removable computer readable medium such as a floppy disk, a compact disk, a magnetic tape, etc. containing control logic and/or data recorded thereon. The computer system may advantageously include or be programmed by appropriate software for reading the control logic and/or the data from the data storage component once inserted in the data retrieving device.

In some embodiments, the computer system may further comprise an expression pattern comparer for comparing the expression pattern(s) stored on a computer readable medium to expression pattern(s) stored on a computer readable medium. An “expression pattern comparer” refers to one or more programs which are implemented on the computer system to compare a nucleotide sequence with other nucleotide sequences. Similarly, programs capable of comparing methylation status patterns and chromatin status patterns are also contemplated by the present invention.

This invention also provides for a computer program that correlates expression patterns with a particular stage of cancer. Similarly, the present invention also provides a computer program that correlates methylation patterns with a particular stage of cancer. Also provided is a computer program that correlates chromatin status with a particular stage of cancer. The computer programs of this invention can optionally include treatment options or drug indications for subjects with expression patterns associated with cancer or the risk of developing cancer.

The present invention is more particularly described in the following examples which are intended as illustrative only since numerous modifications and variations therein will be apparent to those skilled in the art.

### EXAMPLES

Expression Changes

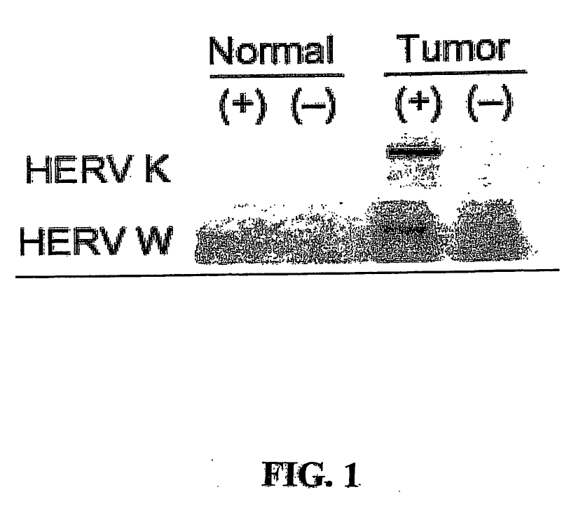

Semi-quantitative RT-PCR was performed to quantify changes in expression from different HERV families, as well as LINEs and SINEs, amongst a small set of malignant, benign, and borderline tumors and non-cancerous ovarian tissue samples. FIG. 1 shows the upregulation of HERV-K and HERV-W families in a cancer sample, compared with a non-cancer sample.

Methylation Status

Methylation levels of HERV-W, and L1 were compared among different ovarian samples. Ten micrograms of genomic DNA were digested either with a methylation sensitive restriction enzyme (HpaII) or with its methylation insensitive isoschizomer (MspI). These enzymes recognize the palindromic sequence CCGG, which is found in diverse positions in the promoter regions of these retroelements. Digestion is carried out overnight at 37° C. with 10 to 16 excess of needed enzyme to ensure complete digestion of the DNA. A control for DNase contamination is included by incubating the same amount of DNA with buffer and water without the enzyme. Digested DNA is run on an agarose gel and transferred to a nylon membrane with NaOH. Membranes are then prehybridized for 1 hour with 10 mg of herring sperm DNA per every milliliter of Church buffer, and hybridized overnight at 65° C. with probes for HERV-K, HERV-W or L1 respectively.

Probe design was based on the hypothesis that relevant DNA methylation changes, if any, would include the predicted promoter regions of retrotransposons.

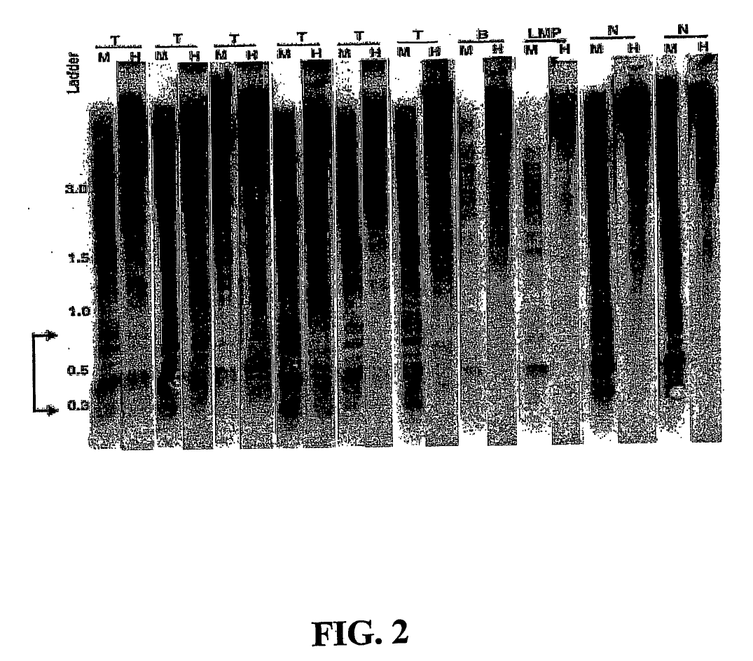

FIG. 2 shows the results obtained after using a probe for the promoter region of HERV-W. After digestion with MspI different bands with approximately the same sizes are observed in cancer, benign, borderline (LMP) and non-cancerous (Non-Cr) samples. After digestion with the methylation sensitive restriction enzyme HpaII, the bands are weaker but still present in most of the cancer samples, while most of the bands, and specially the smaller ones, are absent in the benign, borderline and non-cancerous samples. This result indicates that some methylation has been lost in the cancer samples.

Southern Blot Analysis, LINE1 Probe

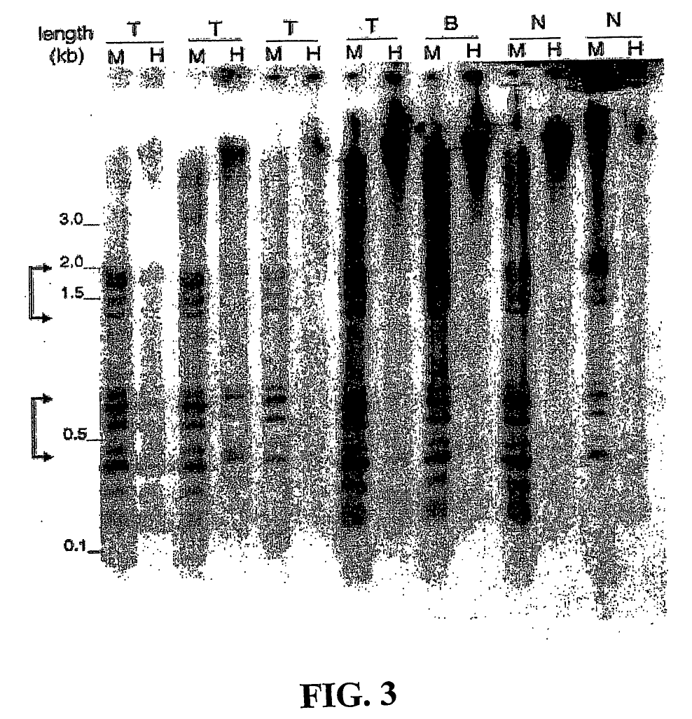

FIG. 3 shows a Southern blot analysis of genomic DNA after digest with MspI (M) or its methylation-sensitive isoschizomer HpaIII (H), resp., hybridized with a LINE1 probe spanning the putative promoter region of the element. Equal amounts of DNA were loaded per sample, i.e. per MspI/HPaII pair. Fragment sizes range from 0.1 kb to >3.0 kb. Samples represent ovarian carcinoma (T—malignant), borderline ovarian tumor (B) and non-tumor ovarian tissue (N).

Fragments between 1.4-2 kb as well as 0.4-0.7 kb (arrows) in HpaIII digests appear more pronounced in the malignant tissue samples compared to the non-tumor samples, indicating extensive cytosine methylation of this particular LINE1 region in non-carcinoma ovarian tissue and loss of LINE1 methylation in some ovarian carcinoma samples.

Southern Blot images are consistent with hypomethylation of Herv-W and LINE1 elements, respectively, in ovarian carcinoma versus normal ovarian tissue. The changes are more pronounced for Herv-W and more consistent among carcinoma samples. There is some heterogeneity for the effect among the samples tested, which will be correlated with clinical history of the tumors and treatment responses.

### Example II

Wide-spread hypomethylation of CpG dinucleotides is characteristic of many cancers. Retrotransposons have been identified as potential targets of hypomethylation during cellular transformation. The following example provides the results of an examination of the methylation status of CpG dinucleotides associated with the L1 and HERV-W retrotransposons in benign and malignant human ovarian tumors. A reduction in the methylation of CpG dinucleotides was found within the promoter regions of these retroelements in malignant relative to non-malignant ovarian tissues. Consistent with these results, it was also found that relative Li and human endogenous retrovirus-W (HERV-W) expression levels are elevated in representative samples of malignant vs. non-malignant ovarian tissues.

The results of a preliminary examination of the methylation status of CpG dinucleotides associated with two representative families of retrotransposons in benign and malignant human ovarian tumors is provided herein. L1 is the most abundant family of human LINE elements comprising about 17% of the genome [22]. Human Endogenous Retrovirus-W (HERV-W) is a family LTR retrotransposons consisting of ˜140 full-length or truncated elements randomly dispersed throughout the human genome [23]. These results demonstrate that large numbers of both families of retrotransposons are hypomethylated in ovarian carcinomas. It is further demonstrated that relative levels of both L1 and HERV-W expression are elevated in representative samples of malignant vs. non-malignant ovarian tissues. The findings presented herein are consistent with the hypothesis that retrotransposons are a major target of global hypomethylation associated with cellular transformation.

To test the hypothesis that L1 and HERV-W elements may experience reduced methylation in malignant ovarian carcinomas, a restriction-enzyme based assay was utilized to compare the methylation status of CpG dinucleotides located within the promoter regions of these elements in a series of malignant and non-malignant ovarian tissues. The restriction enzymes MspI and HpaII both recognize the sequence CCGG but HpaII only cuts when the recognition sequence is unmethylated at the inner cytosine (i.e., CCGG) while MspI is indifferent to the methylation status of the inner cytosine

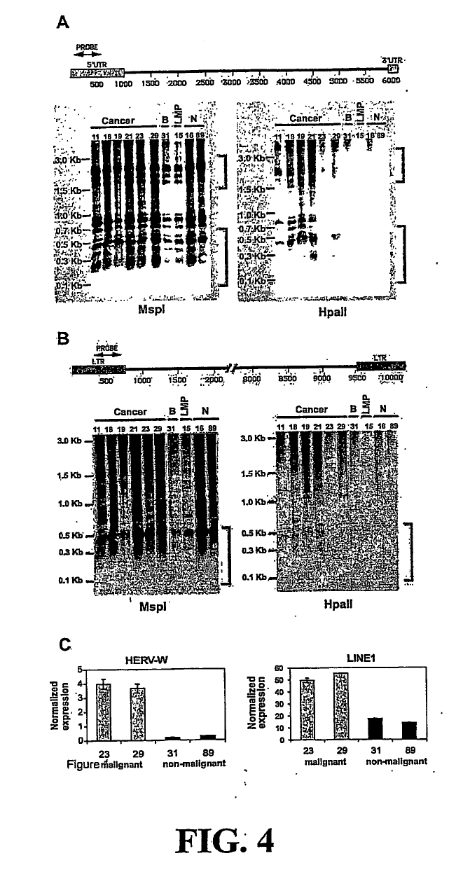

FIGS. 4A & B displays Southern blots of HpaI and MspI digested genomic DNA isolated from tissue samples and hybridized against probes homologous to regions encompassing the promoter regions of each family of elements. The HpaII/MspI restriction sites located within the promoter regions of both L1 and HERV-W elements are polymorphic among family members. By aligning the promoter regions of both families of elements present in the consensus human genome [http://genome.ucsc.edu/] and identifying the HpaII/MspI sites present, it was estimated that the expected size range of restriction fragments within the elements to be between ˜100-700 bp and ˜1500-3000 bp for L1 elements and between ˜100-500 bp for HERV-W elements. Larger sized fragments representing partial digestions and/or polymorphic HpaII/MspI sites located within the elements or in regions flanking the elements are also visible.

The results presented in FIGS. 4A & B show that MspI-generated bands within the expected size range of internal fragments were visible in digestions of DNA from all tissue samples. In contrast, HpaII-generated fragments within the expected size range were only visible in digestions of DNA from the malignant samples. These results are indicative of a consistent reduction in the methylation of CpG dinucleotides within the promoter regions of both L1 and HERV-W elements in the malignant tissue. The fact that the number and intensity of HpaII generated bands in the malignant samples is significantly less than generated by MspI digestion indicates that some L1 and HERV-W elements remain hypermethlyated in the malignant samples. Regardless, this is the first report of the hypomethylation of L1 elements in ovarian carcinomas and of the hypomethylation of HERV-W in any human cancer.

As noted above, hypomethylation of retroelement promoter regions can be expected to result in a localized relaxation of chromatin structure and a corresponding increased element expression [e.g., 10]. In order to test this prediction in these samples, total RNA was extracted from representative samples of two malignant and two non-malignant ovarian tissues and quantitative Real Time RT-PCR was conducted. Two replicate assays were run for each tissue sample. The results shown in FIG. 4C indicate a significant average increase in both L1 and HERV-W expression in the malignant vs. non-malignant ovarian tissues examined.

Hypomethylation is generally associated with the relaxation of chromatin structure, an increased accessibility of transcription factors and a consequent elevation in levels of expression [27]. These findings are generally consistent with these prior results. Since transcription is a rate limiting step in retrotransposition [11], hypomethylation might be expected to result in an increase in retrotransposon insertion mutations. While there have been occasional reports of L1 and other retrotransposon insertion mutations implicated in cancer development in humans [e.g., 28], this may not be as significant a factor as it apparently is in the mouse [29], perhaps because most L1 and other retrotransposon sequences in the human genome are believed to be truncated or otherwise transpositionally defective [30].

Another possible consequence of the hypomethylation of retroelements in humans is the opportunity it provides for ectopic pairing and recombination among homologous elements dispersed throughout the genome. The unequal-crossover events typically associated with ectopic recombination might well account for at least some of the various chromosomal aberrations and aneuploid events characteristic of human malignancies. Indeed, direct evidence of such an effect has recently been documented in mice [31, 32]. In humans, L1 retrotransposition events have been shown to induce various forms of chromosomal instabilities [33] and L1 and other retrotransposon sequences have frequently been linked with a variety of chromosomal aberrations associated with human cancers [e.g., 34].

A third possible consequence of the hypomethylation of retroelements in cancer cells is the potential regulatory impact of the release of methylation complexes known to be bound to these elements in post-embryonic somatic cells [e.g., 35]. Although little is currently understood concerning the factors that determine the relative affinity of methylation complexes for DNA target sequences, retrotransposons are known to be high affinity targets [e.g., 10]. Complexes released from retroelements may initiate a cascade of regulatory changes by binding to other lower affinity target sites and possibly resulting in the down regulation of genes essential for DNA repair and genome stability.

Tissue Samples, DNA Extraction, Southern Hybridization

Bulk ovarian tissue samples were surgically removed and placed in RNA later (Ambion, Austin, Tex.) in the operating room within 1 minute of removal from the patients. The pathological and clinical information of each sample is as follows: Sample #11 (Age 43), Adenocarcinoma (papillary serous, poorly differentiated, Stage IIc); Sample #18 (Age 34), Adenocarcinoma (endometroid, well differentiated, Stage IIb); Sample #19 (Age 57), Adenocarcinoma (papillary serous, poorly differentiated, Stage IIc); Sample #21 (Age 80), Malignant mixed mullerian; Sample #23 (Age 52), Adenocarcinoma (papillary serous, poorly differentiated, Stage IIa); Sample #29 (Age 66), Adenocarcinoma (papillary serous, poorly differentiated, Stage III); Sample #15 (Age 54), Serous borderline /low-malignancy potencial; Sample #31 (Age 40), Benign cystic masses; Sample #16 (Age 53), Normal ovary; Sample #89 (Age 53), Normal ovary. This study was approved by the Institutional Review Board of the University of Georgia and of Northside Hospital (Atlanta), from which the samples were obtained.

Genomic DNA was extracted by proteinase K digestion of 20-25 mg of bulk ovarian tissue and phenol-chlorophorm extraction. DNA was ethanol precipitated and re-suspended in water. Ten micrograms of genomic DNA were digested overnight at 37° C. with 10 to 16 excess amount of either HpaII [methylation sensitive restriction enzyme] or MspI [not sensitive for methylation at internal cytosine]. These enzymes recognize the sequence CCGG, which is found in diverse positions in the promoter regions of these retroelements. Digested DNA was resolved on an agarose gel and transferred to a nylon membrane (Hybond N; Amersham-Biosciences, Piscataway, N.J.) with NaOH. Membranes were prehybridized for 1 hour with 10 mg/ml of herring sperm DNA in Church buffer [0.5M NaH₂PO₄, 7% SDS and 10M EDTA] and hybridized overnight at 65° C. in the same buffer with 100-200 ng of probe DNA labeled with [α-³²P]dCTP using a Nick Translation Kit (Roche, Indianapolis, Ind.). Filters were washed twice for 15 min in 2×SSC and 0.1% SDS and then twice for 30 min in 1×SSC and 0.1% SDS at 65° C. and exposed to Phosphorimager screens (Molecular Dynamics, Sunnyvale, Calif.).

The HERV-W probe was designed in the LTR region, downstream of the putative TTAAAT box. PCR was performed on genomic DNA with forward primer HERVF 5′-CCACCACTGCTGTTTGCCAC-3′ (SEQ ID NO: 771) and reverse primer HERVR 5′-GCCTCGTGTTCTCTGACCTGGGG-3′ (SEQ ID NO: 772), producing a 304 bp fragment. The LINE1 probe for the promoter region was designed according to Takai et al [18]. PCR was performed on genomic DNA with forward primer L1F 5′-CGGGTGATTTCTGCATTTCC-3′ (SEQ ID NO: 773) and reverse primer L1R 5′-GACATTTAAGTCTGCAGAGG-3′ (SEQ ID NO: 774), giving a product of 540 bp. PCR products were cloned into pCR2.1-TOPO and transformed into TOP10 E. coli cells (Invitrogen, Carlsbad, Calif.). Plasmids were extracted (Qiaprep Spin Miniprep Kit, Qiagen, Valencia, Calif.) and sequenced. Subsequent PCR reactions were performed on cloned plasmid DNA for both HERV-W and LINE1, and gel extracted PCR products were used as hybridization probes.

RNA Extraction, Quantitative Real Time RT-PCR

Total RNA was extracted using Trizol Reagent (Invitrogen, Carlsbad, Calif.) and 2-5 μg of total RNA were reverse transcribed into first-strand cDNA using the Thermoscript RT-PCR system (Invitrogen, Carlsbad, Calif.) in a final volume of 20 μl. The HERV-W primers used were: forward; 5′-TTGGCGGTATCACAACCTCT-3′ (SEQ ID NO: 775) reverse; 5′-GTGACGATTCCGGATTGA-3′ (SEQ ID NO: 776); (product size:230 bp) based on the HERV-W sequence (GeneBank accession no. AC000064). The LINE-1 primers were: forward 5′-TCATAAAGCAAGTCCTCAGTGACC-3′ (SEQ ID NO: 777); reverse 5′-GGGGTGGAGAGTTCTGTAGATGTC-3′ (SEQ ID NO: 778) (product size:165 bp) based on the LINE-1 sequence (GeneBank accession no. M80343). Real-time monitoring of PCR reactions was performed using the DNA Engine Opticon 2 System (MJ Research, Waltham, Mass.) and the SYBR Green iQ dye (BioRad, Hercules, Calif.) [24]. For each reaction, the amount of a target and of an endogenous control (Ribosomal Protein S27A) were determined using a calibration curve and the amount of target molecule was divided by the amount of endogenous reference to obtain a normalized target value [25]. RPS27A has been previously identified as a valid control gene in expression studies conducted among human malignant and control tissues [26]. In addition, microarray analyses were utilized to independently verify that RPS27A expression levels are constant among the samples examined in this study. Separate calibration (standard) curves for RPS27A, HERV-W and LINE-1 were constructed using serial dilutions of total cDNA from normal human ovarian tissue (purchased from Ambion, Austin, Tex.). Standards for HERV-W, LINE-1 and RPS27A were defined to contain an arbitrary starting concentration, and serial dilutions were used to construct the standard curve. Standard curve calibrations were included in each assay.

Microarray Analysis of Cancer Cells

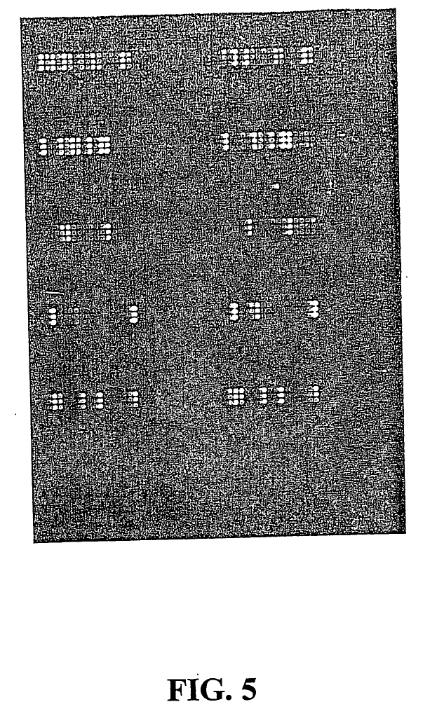

Table 2 shows a ranking of relative retroelement expression values comparing benign (control) vs. malignant (cancer) samples obtaining via microarray analysis on a gene chip (FIG. 5). The results of this experiment show that some retroelement families show a significant increase in expression in cancer (Stage m ovarian carcinoma) vs. controls (negative values in Comparison Rank column), some show no net change (values in Comparison Rank column around 0) and some show a decrease in net levels (positive value in Comparison Rank column). The changes in expression can be due to changes in chromatin structure. Thus, this data set shows that there is a heterogeneous response in changes in chromatin structure in stage III tumors. This example utilizing stage m tumor samples is not limited to a particular stage of type of cancer and is merely illustrative of the kind of changes in retroelement expression that can be analyzed by the methods of the present invention in order to diagnose, stage and treat any type of cancer.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

TABLE 2

| Genename | | B77log | B53log | C141log | C154log | Comparison Rank |
|---|---|---|---|---|---|---|
| L1ME1 | LINE1, ME1 subfamily | 1.35077862 | 1.78180622 | 1.69332148 | 1.64623708 | −0.306105083 |
| ALU_C | SINE element | 0.68972892 | 0.9183396 | 0.80555819 | 0.87181976 | −0.166204761 |
| LTR5_C | long terminal repeat | 1.94516871 | 1.56669724 | 1.03574106 | 1.95720687 | 0.282267811 |
| L1MA4A | LINE1, MA4A subfamily | 1.55470712 | 2.1847541 | 1.72191098 | 1.71634687 | 0.335083736 |
| HERVL74 | Human endogenous retrovirus, subfamily L | 2.1348742 | 1.70081483 | 1.97225587 | 0.94321787 | 0.444734906 |
| L1MD1_5_B | LINE1, MD1 subfamily | 1.72196204 | 2.2003511 | 1.81762843 | 1.58184923 | 0.517665856 |
| MIR3_C | SINE element | 2.1814338 | 1.89379992 | 1.94937867 | 1.54700864 | 0.593194055 |
| L1MB3_5 | LINE1, MB3 subfamily | 2.2090425 | 1.633133 | 1.65469321 | 1.42120887 | 0.669435686 |
| L1PREC2_C | LINE1, PREC2 subfamily | 2.55292039 | 2.16451509 | 2.15268908 | 1.39347057 | 0.721679935 |
| HERV17_C | Human endogenous retrovirus, subfamily 17 | 2.96503482 | 1.86327413 | 1.81145688 | 1.18631188 | 0.749541436 |
| TIGGER2_C | DNA transposon | 2.36529271 | 1.63334668 | 1.52355074 | 1.33672167 | 0.876108867 |
| ZAPHOD | DNA transposon | 2.1513326 | 1.7663077 | 1.64906155 | 1.3920269 | 0.965355576 |
| SVA_C | SINE-R (non retroviral retrotransposon) | 2.2227769 | 1.89286675 | 1.73386684 | 1.30913517 | 1.005075735 |
| HERVE_C | Human endogenous retrovirus, subfamily E | 2.45155247 | 1.77868979 | 1.61843377 | 1.53897952 | 1.008357796 |
| LTR68 | long terminal repeat | 2.34333093 | 2.07355412 | 1.93739866 | 1.63957228 | 1.04634535 |
| CHARLIE3_C | DNA transposon | 2.35703636 | 1.70038524 | 1.48926233 | 1.37092819 | 1.092369458 |
| L1PA2_C | LINE1, PA2 subfamily | 2.16239562 | 2.31209291 | 1.97830497 | 1.45958445 | 1.096598938 |
| THE1A_C | MalR-mammalian LTR retrotransposon | 2.00541667 | 1.93515248 | 1.74245596 | 1.15032661 | 1.118514825 |
| HERVK_C | Human endogenous retrovirus, subfamily K | 2.0061171 | 2.15653499 | 1.82253452 | 1.40105752 | 1.161079999 |
| L1_C | LINE1 | 2.49301356 | 2.34060322 | 2.02819922 | 1.25668997 | 1.185293378 |
| L3_C | LINE3 | 2.35638086 | 2.00908158 | 1.74395501 | 1.54420679 | 1.392505357 |
| MLT2A1_C | MalR-mammalian LTR retrotransposon | 2.40138399 | 2.03382426 | 1.77178165 | 1.60782029 | 1.404321263 |
| L1MC3_C | LINE1, MC3 subfamily | 2.40070124 | 2.12369076 | 1.75851006 | 1.38915384 | 1.506101383 |
| HAL1B | non-autonomous derivative of LINE1 | 2.24611928 | 2.11701552 | 1.76240173 | 1.29920584 | 1.553805998 |
| LTR17_C | terminal repeat | 1.83016919 | 1.99673012 | 1.70364718 | 1.66104849 | 1.562573711 |
| MER74C | MalR-mammalian LTR retrotransposon | 2.10832145 | 2.03572708 | 1.61778714 | 1.04521613 | 1.623238292 |
| L1PA7_C | LINE1, PA7 subfamily | 2.36314897 | 2.35395921 | 1.96388533 | 1.42191829 | 1.707997573 |
| LTR6A | long terminal repeat | 1.86476687 | 2.15684185 | 1.54696871 | 1.4465473 | 1.852173244 |
| MER119 | non-autonomous retroelement | 2.08618876 | 1.8328609 | 1.55129333 | 1.51283891 | 2.071811546 |
| HERVL_C | Human endogenous retrovirus, subfamily L | 2.39027926 | 2.12124503 | 1.74133356 | 1.64196556 | 2.165501757 |
| TIGGER1_C | DNA transposon | 2.07714571 | 2.0604822 | 1.80109953 | 1.57511768 | 2.218870626 |
| MIR_C | mammalian-wide interspersed repeat | 2.1449389 | 2.2361877 | 1.82011015 | 1.62411927 | 2.3063887 |
| THE1BR_C | MalR-mammalian LTR retrotransposon | 2.0698519 | 2.07895536 | 1.72412613 | 1.67293527 | 8.816162784 |

Ranking of genes as computed by the noise to signal ratio derived from mean expression levels at three positions derived from mean expression levels at three positions on a log2 scale: Differential expression between cancer and benign and benign

### REFERENCES

1. Bird A P, Taggart M H: Variable patterns of total DNA and rDNA methylation in animals. Nucleic Acids Res 1980, 8:1485-1497.
2. Whitelaw E, Martin D I: Retrotransposons as epigenetic mediators of phenotypic variation in mammals. Nat Genet 2001, 27:361-365.
3. Robertson K D, Jones P A: DNA methylation: past, present and future directions. Carcinogenesis 2000, 21:461-467.
4. Esteller M, Herman J G: Cancer as an epigenetic disease: DNA methylation and chromatin alterations in human tumours. J Pathol 2002, 196:1-7.
5. Tycko B: DNA and alterations in cancer: genetic and epigenetic alterations. In: Edited by M E. pp. 333-349: Natick: Eaton-Publishing; 2000: 333-349.
6. Ehrlich M: DNA methylation in cancer: too much, but also too little. Oncogene 2002, 21:5400-5413.
7. Jones P A, Baylin S B: The fundamental role of epigenetic events in cancer. Nat Rev Genet 2002, 3:415-428.
8. Qu G, Dubeau L, Narayan A, Yu M C, Ehrlich M: Satellite DNA hypomethylation vs. overall genomic hypomethylation in ovarian epithelial tumors of different malignant potential. Mutat Res 1999, 423:91-101.
9. Florl A R, Lower R, Schmitz-Drager B J, Schulz W A: DNA methylation and expression of LINE-1 and HERV-K provirus sequences in urothelial and renal cell carcinomas. Br J Cancer 1999, 80:1312-1321.
10. Lorincz M C, Schubeler D, Groudine M: Methylation-mediated proviral silencing is associated with MeCP2 recruitment and localized histone H3 deacetylation. Mol Cell Biol 2001, 21:7913-7922.
11. Deininger P L, Batzer M A: Mammalian retroelements. Genome Res 2002, 12:1455-1465.
12. Lander E S, Linton L M, Birren B, Nusbaum C, Zody M C, Baldwin J, Devon K, Dewar K, Doyle M, FitzHugh W, Funke R, Gage D, Harris K, Heaford A, Howland J, Kann L, Lehoczky J, LeVine R, McEwan P, McKernan K, Meldrim J, Mesirov J P, Miranda C, Morris W, Naylor J, Raymond C, Rosetti M, Santos R, Sheridan A, Sougnez C, Stange-Thomann N, Stojanovic N. Subramanian A, Wyman D, Rogers J, Sulston J, Ainscough R, Beck S, Bentley D, Burton J, Clee C, Carter N, Coulson A, Deadman R, Deloukas P, Dunham A, Dunham I, Durbin R, French L, Grafham D, Gregory S, Hubbard T, Humphray S, Hunt A, Jones M, Lloyd C, McMurray A, Matthews L, Mercer S, Milne S, Mullikin J C, Mungall A, Plumb R, Ross M, Shownkeen R, Sims S, Waterston R H, Wilson R K, Hillier L W, McPherson J D, Marra M A, Mardis E R, Fulton L A, Chinwalla A T, Pepin K H, Gish W R, Chissoe S L, Wendl M C, Delehaunty K D, Miner T L, Delehaunty A, Kramer J B, Cook L L, Fulton R S, Johnson D L, Minx P J, Clifton S W, Hawkins T, Branscomb E, Predid P, Richardson P, Wenning S, Slezak T, Doggett N, Cheng J F, Olsen A, Lucas S, Elkin C, Uberbacher E, Frazier M, et al.: Initial sequencing and analysis of the human genome. Nature 2001, 409:860-921.
13. Patzke S, Lindeskog M, Munthe E, Aasheim H C: Characterization of a novel human endogenous retrovirus, HERV-H/F, expressed in human leukemia cell lines. Virology 2002, 303:164-173.
14. Depil S, Roche C, Dussart P, Prin L: Expression of a human endogenous retrovirus, HERV-K, in the blood cells of leukemia patients. Leukemia 2002, 16:254-259.
15. Andersson A C, Svensson A C, Rolny C, Andersson G, Larsson E: Expression of human endogenous retrovirus ERV3 (HERV-R) mRNA in normal and neoplastic tissues. Int J Oncol 1998, 12:309-313.
16. Debniak T, Gorski B, Cybulski C, Jakubowska A, Kurzawski G, Kladny J, Lubinski J: Comparison of Alu-PCR, microsatelite instability, and immunohistochemical analyses in finding features characteristic for hereditary nonpolyposis colorectal cancer. J Cancer Res Clin Oncol 2001, 127:565-569.
17. Wang-Johanning F, Frost A R, Jian B, Epp L, Lu D W, Johanning G L: Quantitation of HERV-K env gene expression and splicing in human breast cancer. Oncogene 2003, 22:.1528-1535.
18. Takai D, Yagi Y, Habib N, Sugimura T, Ushijima T: Hypomethylation of LINE1 retrotransposon in human hepatocellular carcinomas, but not in surrounding liver cirrhosis. Jpn J Clin Oncol 2000, 30:306-309.
19. Santourlidis S, Florl A, Ackermann R. Wirtz, H C, Schulz W A: High frequency of alterations in DNA methylation in adenocarcinoma of the prostate. Prostate 1999, 39:166-174.
20. Dante R, Dante-Paire J, Rigal D, Roizes G: Methylation patterns of long interspersed repeated DNA and alphoid repetitive DNA from human cell lines and tumors. Anticancer Res 1992, 12:559-563.
21. Jurgens B, Schmitz-Drager B J, Schulz W A: Hypomethylation of L1 LINE sequences prevailing in human urothelial carcinoma. Cancer Res 1996, 56:5698-5703.
22. Ostertag. E M, Kazazian H H, Jr.: Biology of mammalian L1 retrotransposons. Annu Rev Genet 2001, 35:501-538.
23. Kim H S, Lee W H: Human endogenous retrovirus HERV-W family:

chromosomal localization, identification, and phylogeny. AIDS Res Hum Retroviruses 2001, 17:643-648.
24. Wittwer C T, Herrmann M G, Moss A A, Rasmussen R P: Continuous fluorescence monitoring of rapid cycle DNA amplification. Biotechniques 1997, 22:130-131, 134-138.
25. Bieche I, Onody P, Laurendeau I, Olivi M, Vidaud D, Lidereau R, Vidaud M: Real-time reverse transcription-PCR assay for future management of ERBB2-based clinical applications. Clin Chem 1999-45:1148-1156.
26. Lee P D, Sladek R, Greenwood C M, Hudson T J: Control genes and variability:

absence of ubiquitous reference transcripts in diverse mammalian expression studies. Genome Res 2002, 12:292-297.
27. Chandler L A, Jones P A: Hypomethylation of DNA in the regulation of gene expression. Dev Biol (N Y 1985) 1988, 5:335-349.
28. Miki Y, Nishisho I, Horii A, Miyoshi Y, Utsunomiya J, Kinzler K W, Vogelstein B, Nakamura Y: Disruption of the APC gene by a retrotransposal insertion of L1 sequence in a colon cancer. Cancer Res 1992, 52:643-645.
29. Kuff E L: Intracisternal A particles in mouse neoplasia. Cancer Cells 1990, 2:398-400.
30. Sassaman D M, Dombroski B A, Moran J V, Kimberland M L, Naas T P, DeBerardinis R J, Gabriel A, Swergold G D, Kazazian H H, Jr.: Many human L1 elements are capable of retrotransposition. Nat Genet 1997, 16:37-43.
31. Eden A, Gaudet F, Wabhmare A, Jaenisch R: Chromosomal instability and tumors promoted by DNA hypomethylation. Science 2003, 300:455-455.
32. Gaudet F, Hodgson J G, Eden A, Jackson-Grusby L, Dausman J, Gray J W, Leonhardt H, Jaenisch R: Induction of tumors in mice by genomic hypomethylation. Science 2003, 300:489-492.
33. Symer D E, Connelly C, Szak S T, Caputo E M, Cost G J, Parmigiani G, Boeke J D: Human I1 retrotransposition is associated with genetic instability in vivo. Cell 2002, 110:327-338.
34. Kolomietz E, Meyn M S, Pandita A, Squire J A: The role of Alu repeat clusters as mediators of recurrent chromosomal aberrations in tumors. Genes Chromosomes Cancer 2002,35:97-112.
35. Hakimi M A, Bochar D A, Schmiesing J A, Dong Y, Barak O G, Speicher D W, Yokomori K, Shiekhattar R: A chromatin remodelling complex that loads cohesin onto human chromosomes. Nature 2002, 418:994-998.

1. A method of determining an expression pattern of one or more families of transposable elements in a sample comprising determining expression of one or more families of transposable elements.

2. A method of assigning an expression pattern of transposable elements to a type of cancerous cell in a sample, comprising:
a) determining expression of one or more families of transposable elements; and
b) assigning the expression pattern obtained from step a) to the type of cancerous cell in the sample.

3. The method of claim 2, wherein the expression pattern is determined by microarray analysis.

4. The method of claim 2, wherein the sample comprises a cell selected from the group consisting of: a carcinoma cell, a fibroma cell, a carcinoma cell, a sarcoma cell, a teratoma cell, and a blastoma cell.

5. The method of claim 2, wherein the sample comprises mixed cell types from a tumor.

6. The method of claim 2, wherein the sample comprises a breast tumor cell of epithelial origin.

7. The method of claim 2, wherein the sample comprises an ovarian tumor cell of epithelial, stromal or germ cell origin.

8. The method of any of claims 1 or 2, wherein the transposable elements are retroelements.

9. A method of diagnosing cancer comprising:
a) determining expression of one or more families of transposable elements in a sample to obtain an expression pattern;
b) matching the expression pattern of step a) with a known expression pattern for a type of cancer; and
c) diagnosing the type of cancer based on matching of the expression pattern of with a known expression pattern for a type of cancer.

10. The method of any of claims 1, 2 or 9, wherein the expression pattern is determined by microarray analysis.

11. The method of claim 9, wherein one or more of the families of transposable elements is selected from the group consisting of retroelement families and DNA element families.

12. The method of claim 11, wherein one or more of the families of retroelements is selected from the group consisting of a family of endogenous retroviruses (ERVs), a family of short interspersed nuclear elements (SINES) and a family of long interspersed nuclear elements (LINEs).

13. A method of determining the effectiveness of an anti-cancer therapeutic in a subject comprising:
a) determining expression of one or more families of transposable elements, in a sample obtained from the subject, to obtain a first expression pattern;
b) administering an anti-cancer therapeutic to the subject;
c) determining expression of one or more families of transposable elements in a sample obtained from the subject after administration of an anti-cancer therapeutic to obtain a second expression pattern; and
d) comparing the second expression pattern with the first expression pattern such that if fewer transposable elements are differentially expressed in the second expression pattern as compared to the first expression pattern, the anti-cancer therapeutic is an effective anti-cancer therapeutic.

14. The method of any of claims 1, 2, 9 or 13, wherein expression of the transposable elements is measured by assaying for the mRNA transcribed from the genes or proteins translated from an mRNA transcribed from the genes.

15. The method of any of claims 1, 2, 9 or 13, wherein expression of two or more families of transposable elements is determined and used to form the pattern of expression.

16. A method of determining a methylation pattern of one or more families of transposable elements in a sample comprising determining methylation of one or more families of transposable elements.

17. A method of assigning a methylation pattern of transposable elements to a type of cancerous cell in a sample, comprising:
a) determining methylation of one or more families of transposable elements; and
b) assigning the methylation pattern obtained from step a) to the type of cancerous cell in the sample.

18. The method of claim 17, wherein the sample comprises a cell selected from the group consisting of: a carcinoma cell, a fibroma cell, a carcinoma cell, a sarcoma cell, a teratoma cell, and a blastoma cell.

19. The method of claim 17, wherein the sample comprises mixed cell types from a tumor.

20. The method of claim 17, wherein the sample comprises a breast tumor cell of epithelial origin.

21. The method of claim 17, wherein the sample comprises an ovarian tumor cell of epithelial, stromal or germ cell origin.

22. The method of any of claims 16 or 17, wherein the transposable elements are selected from the group consisting of retroelements and DNA elements.

23. A method of diagnosing cancer comprising:
a) determining methylation of one or more families of transposable elements in a sample to obtain a methylation pattern;
b) comparing the methylation pattern of step a) with a known methylation pattern for a type of cancer; and
c) diagnosing the type of cancer based on matching of the methylation pattern of a) with a known methylation pattern for a type of cancer.

24. A method of determining the effectiveness of an anti-cancer therapeutic in a subject comprising:
a) determining methylation of one or more families of transposable elements, in a sample obtained from the subject, to obtain a first methylation pattern;
b) administering an anti-cancer therapeutic to the subject;
c) determining methylation of one or more families of transposable elements in a sample obtained from the subject after administration of an anti-cancer therapeutic to obtain a second methylation pattern; and
d) comparing the second methylation pattern with the first methylation pattern such that if there is a change in the second methylation pattern as compared to the first methylation pattern, the anti-cancer therapeutic is an effective anti-cancer therapeutic.

25. The method of any of claims 16, 17, 23 or 24, wherein methylation of the transposable element genes is measured by contacting the methylated transposable element gene sequence with an antibody that specifically binds a methylated sequence.

26. The method of any of claims 16, 17, 23 or 24, wherein methylation of the transposable element genes is measured by contacting the methylated transposable element gene sequence with an antibody that specifically binds a methylation complex protein associated with the methylated transposable element gene sequence.

27. The method of any of claims 16, 17, 23 or 24, wherein methylation of the transposable element genes is monitored by enzymatic means.

28. The method of any of claims 16, 17, 23 or 24, wherein methylation of the transposable element genes is monitored by microarray analysis.

29. The method of any of claims 16, 17, 23 or 24, wherein methylation of the transposable element genes is monitored by methylation-specific PCR.

30. The method of any of claims 16, 17, 23 or 24, wherein the methylation of two or more families of transposable elements is determined and used to form the methylation pattern.

31. A method of determining a chromatin status pattern of one or more families of transposable elements in a sample comprising determining chromatin status of one or more families of transposable elements.

32. A method of assigning a chromatin status pattern of transposable elements to a type of cancerous cell in a sample, comprising:
a) determining chromatin status of one or more families of transposable elements; and
b) assigning the chromatin status pattern obtained from step a) to the type of cancerous cell in the sample.

33. The method of claim 32, wherein the sample comprises a cell selected from the group consisting of: a carcinoma cell, a fibroma cell, a carcinoma cell, a sarcoma cell, a teratoma cell, and a blastoma cell.

34. The method of claim 32, wherein the sample comprises mixed cell types from a tumor.

35. The method of claim 32, wherein the sample comprises a breast tumor cell of epithelial origin.

36. The method of claim 32, wherein the sample comprises an ovarian tumor cell of epithelial, stromal or germ cell origin.

37. The method of any of claims 31 or 32, wherein the transposable elements are selected from the group consisting of retroelements and DNA elements.

38. A method of diagnosing cancer comprising:
a) determining the chromatin status of one or more families of transposable elements in a sample to obtain a chromatin status pattern;
b) comparing the chromatin status pattern of step a) with a known chromatin status pattern for a type of cancer; and
c) diagnosing the type of cancer based on matching of the chromatin status pattern of a with a known chromatin status pattern for a type of cancer.

39. A method of determining the effectiveness of an anti-cancer therapeutic in a subject comprising:
a) determining the chromatin status of one or more families of transposable elements, in a sample obtained from the subject, to obtain a first chromatin status pattern;
b) administering an anti-cancer therapeutic to the subject;
c) determining chromatin status of one or more families of transposable elements in a sample obtained from the subject after administration of an anti-cancer therapeutic to obtain a second chromatin status pattern; and
d) comparing the second chromatin status pattern with the first chromatin status pattern such that if there is a change in the second chromatin status pattern as compared to the first chromatin status pattern, the anti-cancer therapeutic is an effective anti-cancer therapeutic.

40. The method of any of claims 31, 32, 38 or 39, wherein chromatin status of the transposable element genes is measured by determining the accessibility of transposable element genes to a restriction enzyme.

41. The method of any of claims 31, 32, 38 or 39, wherein chromatin status of the transposable element genes is monitored by microarray analysis.

42. The method of any of claims 31, 32, 38 or 39, wherein the chromatin status of two or more families of transposable elements is determined and used to form the chromatin status pattern.
